(19) United States
(12) Patent Application Publication
(10) Pub. No.: US 2024/0148797 A1
(43) Pub. Date: May 9, 2024

(54) COMPOSITIONS AND METHODS FOR REDUCING CYTOKINE EXPRESSION

(71) Applicant: EVELO BIOSCIENCES, INC., Cambridge, MA (US)

(72) Inventors: David Epstein, Sunny Isles Beach, FL (US); Duncan McHale, Kent (GB)

(21) Appl. No.: 18/278,968

(22) PCT Filed: Feb. 23, 2022
(86) PCT No.: PCT/US2022/017446

§ 371 Date: Aug. 25, 2023

Related U.S. Application Data
(60) Provisional application No. 63/154,218, filed on Feb. 26, 2021.

(51) Int. Cl. A61K 35/74; A61K 9/127; A61K 45/06; A61P 31/14

(52) U.S. Cl. CPC A61K 35/74; A61K 9/127; A61K 45/06; A61P 31/14

(57) ABSTRACT

Provided herein are methods and compositions related to Prevotella histicola extracellular vesicles (EVs), and solutions and dried forms (and therapeutic compositions thereof) of Prevotella histicola extracellular vesicles (EVs) for the reduction of IL-8, IL-6, IL-1β, and/or TNFα expression and/or for the treatment of viral infections.

### CROSS-REFERENCE TO RELATED APPLICATION

This application claims the benefit of U.S. Provisional Application Ser. No. 63/154,218, filed Feb. 26, 2021, the entire contents of which are incorporated herein by reference.

### BACKGROUND

Inflammation can be a protective response to harmful stimuli, such as invading pathogens, damaged cells, toxic compounds, or cancerous cells. However excessive inflammatory responses to such stimuli can result in serious adverse effects, including tissue damage and even death. For example, production of pro-inflammatory cytokines such as interleukin-8 (IL-8), interleukin-6 (IL-6), interleukin-1 beta (IL-1β), and tumor necrosis factor alpha (TNFα) in response to many viral infections is one of the primary causes of the adverse symptoms associated with infection (including, in some cases, death). For example, release of inflammatory cytokines has been associated with disease severity resulting from infection by a number of viruses, including infection by coronaviruses (e.g., SARS-CoV-2, the virus that causes Coronavirus Disease 2019 (COVID-19)), influenza viruses, and respiratory syncytial viruses. For example, patients with severe COVID-19 often exhibit elevated levels of inflammatory cytokines in their lungs, which contributes to lung damage experienced by the COVID-19 patients.

Thus, there is a great need for new compositions and methods for the reduction of inflammatory cytokine expression, particularly in subjects who have been infected by a respiratory virus and/or who have an increased risk of being infected by a respiratory virus.

### SUMMARY

Therapeutic compositions comprising extracellular vesicles (EVs), such as EVs obtained from Prevotella histicola bacteria, have therapeutic effects and are useful for the treatment and/or prevention of disease and/or health disorders. As described herein, EVs from Prevotella histicola bacteria can be prepared as a biomass (e.g., isolated EVs can be resuspended in a buffer such as PBS). As described herein, EVs from Prevotella histicola bacteria can be prepared as solutions, dried forms and/or therapeutic compositions.

In some embodiments, dried forms having a moisture content below about 6% are better suited for downstream processing. In some embodiments, dried forms having a moisture content below about 6% have improved stability. In some embodiments, the solutions comprising the EVs from Prevotella histicola bacteria also comprise an excipient that contains a bulking agent, and optionally comprises one or more additional ingredients, such as a lyoprotectant. In some embodiments, the solutions comprising the EVs from Prevotella histicola bacteria also comprise an excipient that contains a lyoprotectant, and optionally comprises one or more additional ingredients, such as a bulking agent. In some embodiments, the dried forms comprising the EVs from Prevotella histicola bacteria also comprise an excipient that contains a bulking agent, and that optionally comprises one or more additional ingredients, such as a lyoprotectant. In some embodiments, the dried forms comprising the EVs from Prevotella histicola bacteria also comprise an excipient that contains a lyoprotectant, and optionally comprise one or more additional ingredients, such as a bulking agent.

Provided herein are methods and compositions (e.g., solutions, dried forms and/or therapeutic compositions) related to the use of extracellular vesicles (EVs) from certain Prevotella histicola strains for the reduction of inflammatory cytokine expression (e.g., IL-8, IL-6, IL-1β, and/or TNFα expression) and/or for the treatment of bacterial septic shock, cytokine storm and/or viral infection. In some embodiments, the methods and compositions provided herein are for the reduction of inflammatory cytokine expression (e.g., IL-8, IL-6, IL-1β, and/or TNFα expression) and/or for the treatment of a viral infection such as a respiratory viral infection, such as a coronavirus infection (e.g., a MERS (Middle East Respiratory Syndrome) infection, a severe acute respiratory syndrome (SARS) infection, such as a SARS-CoV-2 infection), an influenza infection, and/or a respiratory syncytial virus infection. In some embodiments, the methods and compositions provided herein are for the treatment of a coronavirus infection (e.g., a MERS infection, a severe acute respiratory syndrome (SARS) infection, such as a SARS-CoV-2 infection). In some embodiments, provided herein are methods of treating COVID-19. In some embodiments, the methods and compositions provided herein are for the treatment of an influenza virus infection.

In certain aspects, provided herein is a method of reducing inflammatory cytokine expression (e.g., reducing IL-8, IL-6, IL-1β, and/or TNFα expression levels) in a subject in need thereof, comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in reducing inflammatory cytokine expression (e.g., reducing IL-8, IL-6, IL-1β, and/or TNFα expression levels) in a subject in need thereof.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for reducing inflammatory cytokine expression (e.g., reducing IL-8, IL-6, IL-1β, and/or TNFα expression levels) in a subject in need thereof.

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain embodiments, provided herein is a method of reducing IL-8 expression levels.

In certain embodiments, provided herein is a method of reducing IL-6 expression levels.

In certain embodiments, provided herein is a method of reducing IL-10 expression levels.

In certain embodiments, provided herein is a method of reducing TNFα expression levels.

In certain embodiments, provided herein is a method of reducing IL-8 and IL-6 expression levels.

In certain embodiments, provided herein is a method of reducing IL-8, IL-6, and TNFα expression levels.

In certain aspects, provided herein is a method of resolving peripheral inflammation without leading to immunosuppression of an anti-viral response (e.g., as described herein) in a subject in need thereof, comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in resolving peripheral inflammation without leading to immunosuppression of an anti-viral response (e.g., as described herein) in a subject in need thereof.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for resolving peripheral inflammation without leading to immunosuppression of an anti-viral response (e.g., as described herein) in a subject in need thereof.

In some embodiments, an anti-viral response comprises one or more of: CD4 and CD8 T cell production of IFN-γ, innate anti-viral production of IFN-α and IFN-β, and/or the generation of effector T cell populations.

In some embodiments, peripheral inflammation comprises elevated IL-8, IL-6, IL-1β, and/or TNFα expression levels (e.g., as compared to a standard).

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain aspects, provided herein is a method of reducing inflammatory cytokine expression (e.g., reducing IL-8, IL-6, IL-1β, and/or TNFα expression levels) in a subject in need thereof, comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles, wherein a Type I interferon response is not reduced (e.g., not reduced to the same extent that the inflammatory cytokine expression is reduced), e.g., as determined by anti-viral TLR3-mediated Type I interferon levels, e.g., as determined by IFNα and/or IFNβ levels, e.g., as described herein.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in reducing inflammatory cytokine expression (e.g., reducing IL-8, IL-6, IL-1β, and/or TNFα expression levels) in a subject in need thereof, wherein a Type I interferon response is not reduced (e.g., not reduced to the same extent that the inflammatory cytokine expression is reduced), e.g., as determined by anti-viral TLR3-mediated Type I interferon levels, e.g., as determined by IFNα and/or IFNβ levels, e.g., as described herein.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for reducing inflammatory cytokine expression (e.g., reducing IL-8, IL-6, IL-1β, and/or TNFα expression levels) in a subject in need thereof, wherein a Type I interferon response is not reduced (e.g., not reduced to the same extent that the inflammatory cytokine expression is reduced), e.g., as determined by anti-viral TLR3-mediated Type I interferon levels, e.g., as determined by IFNα and/or IFNβ levels, e.g., as described herein.

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain aspects, provided herein is a method of reducing inflammatory cytokine expression (e.g., reducing IL-8, IL-6, IL-1β, and/or TNFα expression levels) in a subject in need thereof, comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles, wherein a Type II interferon response is not reduced (e.g., not reduced to the same extent that the inflammatory cytokine expression is reduced), e.g., as determined by Type II interferon levels, e.g., as determined by IFNγ levels, e.g., as described herein.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in reducing inflammatory cytokine expression (e.g., reducing IL-8, IL-6, IL-1β, and/or TNFα expression levels) in a subject in need thereof, wherein a Type II interferon response is not reduced (e.g., not reduced to the same extent that the inflammatory cytokine expression is reduced), e.g., as determined by Type II interferon levels, e.g., as determined by IFNγ levels, e.g., as described herein.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for reducing inflammatory cytokine expression (e.g., reducing IL-8, IL-6, IL-1β, and/or TNFα expression levels) in a subject in need thereof, wherein a Type II interferon response is not reduced (e.g., not reduced to the same extent that the inflammatory cytokine expression is reduced), e.g., as determined by Type II interferon levels, e.g., as determined by IFNγ levels, e.g., as described herein.

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain aspects, provided herein is a method of reducing inflammatory cytokine expression (e.g., reducing IL-8, IL-6, IL-1β, and/or TNFα expression levels) in a subject in need thereof, comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles, wherein interferon gamma (IFNγ) production by T cells is not reduced (e.g., not reduced to the same extent that the inflammatory cytokine expression is reduced), e.g., as described herein.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in reducing inflammatory cytokine expression (e.g., reducing IL-8, IL-6, IL-1β, and/or TNFα expression levels) in a subject in need thereof, wherein interferon gamma (IFNγ) production by T cells is not reduced (e.g., not reduced to the same extent that the inflammatory cytokine expression is reduced), e.g., as described herein.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for reducing inflammatory cytokine expression (e.g., reducing IL-8, IL-6, IL-1β, and/or TNFα expression levels) in a subject in need thereof, wherein interferon gamma (IFNγ) production by T cells is not reduced (e.g., not reduced to the same extent that the inflammatory cytokine expression is reduced), e.g., as described herein.

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain aspects, provided herein is a method of reducing inflammatory cytokine expression (e.g., reducing IL-8, IL-6, IL-1β, and/or TNFα expression levels) in a subject in need thereof, comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles, wherein generation of functional CD4+Th1 cells is not reduced (e.g., not reduced to the same extent that the inflammatory cytokine expression is reduced), e.g., as described herein.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in reducing inflammatory cytokine expression (e.g., reducing IL-8, IL-6, IL-1β, and/or TNFα expression levels) in a subject in need thereof, wherein generation of functional CD4+Th1 cells is not reduced (e.g., not reduced to the same extent that the inflammatory cytokine expression is reduced), e.g., as described herein.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for reducing inflammatory cytokine expression (e.g., reducing IL-8, IL-6, IL-1β, and/or TNFα expression levels) in a subject in need thereof, wherein generation of functional CD4+Th1 cells is not reduced (e.g., not reduced to the same extent that the inflammatory cytokine expression is reduced), e.g., as described herein.

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain aspects, provided herein is a method of reducing inflammatory cytokine expression (e.g., reducing IL-8, IL-6, IL-1β, and/or TNFα expression levels) in a subject in need thereof, comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles, wherein production of interferon-gamma from human memory CD8 T cells in response to a viral peptide pool (Cytomegalovirus, Epstein-Bar virus, and Influenza virus) is not reduced (e.g., not reduced to the same extent that the inflammatory cytokine expression is reduced), e.g., as described herein.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in reducing inflammatory cytokine expression (e.g., reducing IL-8, IL-6, IL-1β, and/or TNFα expression levels) in a subject in need thereof, wherein production of interferon-gamma from human memory CD8 T cells in response to a viral peptide pool (Cytomegalovirus, Epstein-Bar virus, and Influenza virus) is not reduced (e.g., not reduced to the same extent that the inflammatory cytokine expression is reduced), e.g., as described herein.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for reducing inflammatory cytokine expression (e.g., reducing IL-8, IL-6, IL-1β, and/or TNFα expression levels) in a subject in need thereof, wherein production of interferon-gamma from human memory CD8 T cells in response to a viral peptide pool (Cytomegalovirus, Epstein-Bar virus, and Influenza virus) is not reduced (e.g., not reduced to the same extent that the inflammatory cytokine expression is reduced), e.g., as described herein.

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain aspects, provided herein is a method of treating a viral infection in a subject comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in treating a viral infection in a subject.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for of treating a viral infection in a subject.

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329). In some embodiments, the viral infection is a coronavirus infection, an influenza infection, and/or a respiratory syncytial virus infection. In some embodiments the viral infection is a SARS-CoV-2 infection.

In certain aspects, provided herein is a method of treating COVID-19 in a subject comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in treating COVID-19 in a subject.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for of treating COVID-19 in a subject.

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain aspects, provided herein is a method of treating cytokine storm syndrome (cytokine release syndrome) (e.g., a cytokine storm resulting from a viral infection, such as a SARS-CoV-2 infection) in a subject comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in treating cytokine storm syndrome (cytokine release syndrome) (e.g., a cytokine storm resulting from a viral infection, such as a SARS-CoV-2 infection) in a subject.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for of treating cytokine storm syndrome (cytokine release syndrome) (e.g., a cytokine storm resulting from a viral infection, such as a SARS-CoV-2 infection) in a subject.

In some embodiments, the extracellular vesicles are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In some embodiments of the methods provided herein, the method improves pulmonary function in the subject, as measured by the change in Oxygen Saturation (SpO2)/Fraction of Inspired Oxygen (FiO2) [S/F] ratio, e.g., as measured by a change from baseline to the lowest S/F ratio measured in days 1-14 as described herein.

In some embodiments of the methods provided herein, the method improves a clinical endpoint in a subject, e.g., an endpoint described herein, e.g., an endpoint provided in Table 1.

In some embodiments of the methods provided herein, the method decreases development of complications of COVID-19 infection, e.g., as described herein.

In some embodiments of the methods provided herein, the method decreases severity of complications of COVID-19 infection, e.g., as described herein.

In some embodiments of the methods provided herein, the method improves the WHO OSCI score in a subject, e.g., evaluated as described herein.

In some embodiments of the methods provided herein, the method decreases length of hospitalization in subjects with COVID-19, e.g., as described herein.

In some embodiments of the methods provided herein, the method decreases length of recovery in subjects with COVID-19, e.g., as described herein.

In some embodiments of the methods provided herein, the method decreases the exaggerated host cytokine response to COVID-19 infection, e.g., as determined by change from baseline in a cytokine level (such as IL-8, IL-6, IL-1β, and/or TNFα) at day 4 and/or day 7 and/or by change from baseline in inflammatory response at day 4 and/or day 7, e.g., as described herein. In some embodiments, the method decreases the exaggerated host cytokine response to COVID-19 infection, e.g., as determined by change from baseline in IL-6 levels at day 4 and/or day 7, e.g., as described herein.

In some embodiments of the methods provided herein, the method causes a change in a biomarker, e.g., a biomarker described herein, e.g., as determined by change from baseline in the biomarker at day 4 and day 7. The biomarker can be, for example, one or more of: differential white cell count, neutrophil to lymphocyte ratio, CRP, IL-6, IL-8, Ferritin, D-Dimer, Troponin, Eotaxin, Eotaxin-3, GM-CSF, IFN-γ, IL-1α, IL-1β, IL-2, IL-4, IL-5, IL-7, IL-8 (HA), IL-10, IL-12/IL-23p40, IL-12p70, IL-13, IL-15, IL-16, IL-17A, IP-10, MCP-1, MCP-4, MDC, MIP-1α, MIP-1β, TARC, TNF-α, TNF-β, and/or VEGF-A levels (e.g., protein or mRNA levels).

Extracellular vesicles (EVs), such as EVs obtained from Prevotella histicola bacteria, have therapeutic effects and are useful for the treatment and/or prevention of disease and/or health disorders. As described herein, EVs from Prevotella histicola bacteria can be prepared as a biomass (e.g., isolated EVs can be resuspended in a buffer such as PBS). As described herein, EVs from Prevotella histicola bacteria can be prepared as solutions, dried forms and/or therapeutic compositions. The solutions, dried forms and/or therapeutic compositions containing the EVs can also include an excipient that contains a bulking agent, and optionally one or more additional components, such as a lyoprotectant. The Prevotella histicola EVs can be used in methods of treatment, e.g., as described herein.

In some embodiments, the EVs are administered orally.

In some embodiments, the EVs are administered intranasally.

In certain aspects, the extracellular vesicles (EVs) from a Prevotella histicola strain and/or a composition (e.g., a solution, dried form and/or therapeutic composition) comprising the extracellular vesicles reduce inflammation in a DTH model of inflammation.

In some embodiments, the dose is in the form of one or more capsules, optionally comprising an enteric-coating (e.g., enteric-coated capsules). In some embodiments, the dose is in the form of one or more tablets, optionally comprising an enteric-coating (e.g., enteric-coated tablets). In some embodiments, the dose is in the form of one or more mini-tablets. In some embodiments, the mini-tablets are enteric-coated mini-tablets. In some embodiments, the dose is in the form of a non-enteric coated capsule comprising one or more enteric-coated mini-tablets.

As disclosed herein, Prevotella histicola extracellular vesicles (EVs) have therapeutic effects and are useful for the treatment and/or prevention of disease and/or health disorders. Therapeutic compositions of biomass, solutions and dried forms containing Prevotella histicola EVs can be prepared.

Bulking agents and/or lyoprotectants are used when preparing extracellular vesicles (EVs) for drying, such as freeze drying and spray drying. In some embodiments, bulking agents, including but not limited to sucrose, mannitol, polyethylene glycol (PEG, such as PEG 6000), cyclodextrin, maltodextrin, and dextran (such as dextran 40k), make dried forms (such as powders and/or lyophilates) easier to handle after drying. In some embodiments, bulking agents improve the properties of a dried form. In some embodiments, lyoprotectants, including but not limited to trehalose, sucrose, and lactose protect the EVs during drying, such as freeze-drying or spray drying. In some embodiments, the excipient functions to decrease drying cycle time. In some embodiments, the excipient functions to maintain therapeutic efficacy of the EVs.

In some aspects, the disclosure provides a dried form comprising extracellular vesicles (EVs) from Prevotella histicola bacteria, wherein the dried form has a moisture content (e.g., as determined by the Karl Fischer method) of below about 6%.

In some embodiments, the dried form provided herein has a moisture content (e.g., as determined by the Karl Fischer method) of below about 5%.

In some embodiments, the dried form provided herein has a moisture content (e.g., as determined by the Karl Fischer method) of below about 4%.

In some embodiments, the dried form provided herein has a moisture content (e.g., as determined by the Karl Fischer method) of between about 1% to about 4%.

In some embodiments, the dried form provided herein has a moisture content (e.g., as determined by the Karl Fischer method) of between about 2% to about 4%.

In some embodiments, the dried form provided herein has a moisture content (e.g., as determined by the Karl Fischer method) of between about 2% to about 3%.

In some aspects, the disclosure provides a lyophilate comprising extracellular vesicles (EVs) from Prevotella histicola bacteria, wherein the lyophilate has a moisture content (e.g., as determined by the Karl Fischer method) of below about 6%.

In some embodiments, the lyophilate has a moisture content (e.g., as determined by the Karl Fischer method) of below about 5%.

In some embodiments, the lyophilate has a moisture content (e.g., as determined by the Karl Fischer method) of below about 4%.

In some embodiments, the lyophilate has a moisture content (e.g., as determined by the Karl Fischer method) of between about 1% to about 4%.

In some embodiments, the lyophilate has a moisture content (e.g., as determined by the Karl Fischer method) of between about 2% to about 4%.

In some embodiments, the lyophilate has a moisture content (e.g., as determined by the Karl Fischer method) of between about 2% to about 3%.

In some aspects, the disclosure provides a powder comprising extracellular vesicles (EVs) from Prevotella histicola bacteria, wherein the powder has a moisture content (e.g., as determined by the Karl Fischer method) of below about 6%.

In some embodiments, the powder has a moisture content (e.g., as determined by the Karl Fischer method) of below about 5%.

In some embodiments, the powder has a moisture content (e.g., as determined by the Karl Fischer method) of below about 4%.

In some embodiments, the powder has a moisture content (e.g., as determined by the Karl Fischer method) of between about 1% to about 4%.

In some embodiments, the powder has a moisture content (e.g., as determined by the Karl Fischer method) of between about 2% to about 4%.

In some embodiments, the powder has a moisture content (e.g., as determined by the Karl Fischer method) of between about 2% to about 3%.

In some aspects, the disclosure provides a dried form comprising extracellular vesicles (EVs) from Prevotella histicola bacteria and an excipient, wherein the excipient comprises about 95% to about 99% of the total mass of the dried form.

In some aspects, the disclosure provides a dried form comprising extracellular vesicles (EVs) from Prevotella histicola bacteria and an excipient, wherein the EVs can make up about 2% to about 6% of the total mass of the dried form.

In some embodiments of the dried form provided herein, the dried form comprises a powder. In some embodiments, the powder comprises a lyophilized powder. In some embodiments the powder comprises a spray-dried powder.

In some embodiments of the dried form provided herein, the dried form comprises a lyophilate. In some embodiments, the lyophilate comprises a lyophilized powder. In some embodiments, the lyophilate comprises a lyophilized cake.

In some aspects, the disclosure provides extracellular vesicles (EVs) from Prevotella histicola bacteria.

In some aspects, the disclosure provides a therapeutic composition comprising the Prevotella histicola EVs, wherein the composition further comprises a pharmaceutically acceptable excipient.

In some aspects, the disclosure provides a solution comprising extracellular vesicles (EVs) from Prevotella histicola bacteria and an excipient that comprises a bulking agent.

In some aspects, the disclosure provides a solution consisting essentially of extracellular vesicles (EVs) from Prevotella histicola bacteria and an excipient that comprises a bulking agent.

In some aspects, the disclosure provides a solution comprising extracellular vesicles (EVs) from Prevotella histicola bacteria and an excipient that comprises a bulking agent and a lyoprotectant.

In some aspects, the disclosure provides a solution consisting essentially of extracellular vesicles (EVs) from Prevotella histicola bacteria and an excipient that comprises a bulking agent and a lyoprotectant.

In some aspects, the disclosure provides a solution comprising extracellular vesicles (EVs) from Prevotella histicola bacteria and an excipient that comprises a lyoprotectant.

In some aspects, the disclosure provides a solution consisting essentially of extracellular vesicles (EVs) from Prevotella histicola bacteria and an excipient that comprises a lyoprotectant.

In some aspects, the disclosure provides a therapeutic composition comprising the solution, wherein the composition further comprises a pharmaceutically acceptable excipient. In some embodiments, the pharmaceutically acceptable excipient comprises a glidant, lubricant, and/or diluent.

In some aspects, the disclosure provides a dried form comprising extracellular vesicles (EVs) from Prevotella histicola bacteria and an excipient that comprises a bulking agent.

In some aspects, the disclosure provides a dried form consisting essentially of extracellular vesicles (EVs) from Prevotella histicola bacteria and an excipient that comprises a bulking agent.

In some aspects, the disclosure provides a dried form comprising extracellular vesicles (EVs) from Prevotella histicola bacteria and an excipient that comprises a bulking agent and a lyoprotectant.

In some aspects, the disclosure provides a dried form consisting essentially of extracellular vesicles (EVs) from Prevotella histicola bacteria and an excipient that comprises a bulking agent and a lyoprotectant.

In some aspects, the disclosure provides a dried form comprising extracellular vesicles (EVs) from Prevotella histicola bacteria and an excipient that comprises a lyoprotectant.

In some aspects, the disclosure provides a dried form consisting essentially of extracellular vesicles (EVs) from Prevotella histicola bacteria and an excipient that comprises a lyoprotectant.

In some aspects, the disclosure provides a therapeutic composition comprising the dried form, wherein the composition further comprises a pharmaceutically acceptable excipient. In some embodiments, the pharmaceutically acceptable excipient comprises a glidant, lubricant, and/or diluent.

In some aspects, the disclosure provides a powder comprising extracellular vesicles (EVs) from Prevotella histicola bacteria and an excipient that comprises a bulking agent.

In some aspects, the disclosure provides a powder consisting essentially of extracellular vesicles (EVs) from Prevotella histicola bacteria and an excipient that comprises a bulking agent.

In some aspects, the disclosure provides a powder comprising extracellular vesicles (EVs) from Prevotella histicola bacteria and an excipient that comprises a bulking agent and a lyoprotectant.

In some aspects, the disclosure provides a powder consisting essentially of extracellular vesicles (EVs) from Prevotella histicola bacteria and an excipient that comprises a bulking agent and a lyoprotectant.

In some aspects, the disclosure provides a powder comprising extracellular vesicles (EVs) from Prevotella histicola bacteria and an excipient that comprises a lyoprotectant.

In some aspects, the disclosure provides a powder consisting essentially of extracellular vesicles (EVs) from Prevotella histicola bacteria and an excipient that comprises a lyoprotectant.

In some aspects, the disclosure provides a therapeutic composition comprising the powder, wherein the composition further comprises a pharmaceutically acceptable excipient. In some embodiments, the pharmaceutically acceptable excipient comprises a glidant, lubricant, and/or diluent.

In some aspects, the disclosure provides a spray-dried powder comprising extracellular vesicles (EVs) from Prevotella histicola bacteria and an excipient that comprises a bulking agent.

In some aspects, the disclosure provides a spray-dried powder consisting essentially of extracellular vesicles (EVs) from Prevotella histicola bacteria and an excipient that comprises a bulking agent.

In some aspects, the disclosure provides a spray-dried powder comprising extracellular vesicles (EVs) from Prevotella histicola bacteria and an excipient that comprises a bulking agent and a lyoprotectant.

In some aspects, the disclosure provides a spray-dried powder consisting essentially of extracellular vesicles (EVs) from Prevotella histicola bacteria and an excipient that comprises a bulking agent and a lyoprotectant.

In some aspects, the disclosure provides a spray-dried powder comprising extracellular vesicles (EVs) from Prevotella histicola bacteria and an excipient that comprises a lyoprotectant.

In some aspects, the disclosure provides a spray-dried powder consisting essentially of extracellular vesicles (EVs) from Prevotella histicola bacteria and an excipient that comprises a lyoprotectant.

In some aspects, the disclosure provides a therapeutic composition comprising the spray-dried powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilate comprising extracellular vesicles (EVs) from Prevotella histicola bacteria and an excipient that comprises a bulking agent.

In some aspects, the disclosure provides a lyophilate consisting essentially of extracellular vesicles (EVs) from Prevotella histicola bacteria and an excipient that comprises a bulking agent.

In some aspects, the disclosure provides a lyophilate comprising extracellular vesicles (EVs) from Prevotella histicola bacteria and an excipient that comprises a bulking agent and a lyoprotectant.

In some aspects, the disclosure provides a lyophilate consisting essentially of extracellular vesicles (EVs) from Prevotella histicola bacteria and an excipient that comprises a bulking agent and a lyoprotectant.

In some aspects, the disclosure provides a lyophilate comprising extracellular vesicles (EVs) from Prevotella histicola bacteria and an excipient that comprises a lyoprotectant.

In some aspects, the disclosure provides a lyophilate consisting essentially of extracellular vesicles (EVs) from Prevotella histicola bacteria and an excipient that comprises a lyoprotectant.

In some aspects, the disclosure provides a therapeutic composition comprising the lyophilate, wherein the composition further comprises a pharmaceutically acceptable excipient. In some embodiments, the pharmaceutically acceptable excipient comprises a glidant, lubricant, and/or diluent.

In some aspects, the disclosure provides a lyophilized powder comprising extracellular vesicles (EVs) from Prevotella histicola bacteria and an excipient that comprises a bulking agent.

In some aspects, the disclosure provides a lyophilized powder consisting essentially of extracellular vesicles (EVs) from Prevotella histicola bacteria and an excipient that comprises a bulking agent.

In some aspects, the disclosure provides a lyophilized powder comprising extracellular vesicles (EVs) from Prevotella histicola bacteria and an excipient that comprises a bulking agent and a lyoprotectant.

In some aspects, the disclosure provides a lyophilized powder consisting essentially of extracellular vesicles (EVs) from Prevotella histicola bacteria and from an excipient that comprises a bulking agent and a lyoprotectant.

In some aspects, the disclosure provides a lyophilized powder comprising extracellular vesicles (EVs) from Prevotella histicola bacteria and an excipient that comprises a lyoprotectant.

In some aspects, the disclosure provides a lyophilized powder consisting essentially of extracellular vesicles (EVs) from Prevotella histicola bacteria and from an excipient that comprises a lyoprotectant.

In some aspects, the disclosure provides a therapeutic composition comprising the lyophilized powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilized cake comprising extracellular vesicles (EVs) from Prevotella histicola bacteria and an excipient that comprises a bulking agent.

In some aspects, the disclosure provides a lyophilized cake consisting essentially of extracellular vesicles (EVs) from Prevotella histicola bacteria and an excipient that comprises a bulking agent.

In some aspects, the disclosure provides a lyophilized cake comprising extracellular vesicles (EVs) from Prevotella histicola bacteria and an excipient that comprises a bulking agent and a lyoprotectant.

In some aspects, the disclosure provides a lyophilized cake consisting essentially of extracellular vesicles (EVs) from Prevotella histicola bacteria and an excipient that comprises a bulking agent and a lyoprotectant.

In some aspects, the disclosure provides a lyophilized cake comprising extracellular vesicles (EVs) from Prevotella histicola bacteria and an excipient that comprises a lyoprotectant.

In some aspects, the disclosure provides a lyophilized cake consisting essentially of extracellular vesicles (EVs) from Prevotella histicola bacteria and an excipient that comprises a lyoprotectant.

In some aspects, the disclosure provides a therapeutic composition comprising extracellular vesicles (EVs) from Prevotella histicola bacteria and an excipient that comprises a bulking agent.

In some aspects, the disclosure provides a therapeutic composition consisting essentially of extracellular vesicles (EVs) from Prevotella histicola bacteria and an excipient that comprises a bulking agent.

In some aspects, the disclosure provides a therapeutic composition comprising extracellular vesicles (EVs) from Prevotella histicola bacteria and an excipient that comprises a bulking agent and a lyoprotectant.

In some aspects, the disclosure provides a therapeutic composition consisting essentially of extracellular vesicles (EVs) from Prevotella histicola bacteria and an excipient that comprises a bulking agent and a lyoprotectant.

In some aspects, the disclosure provides a therapeutic composition comprising extracellular vesicles (EVs) from Prevotella histicola bacteria and an excipient that comprises a lyoprotectant.

In some aspects, the disclosure provides a therapeutic composition consisting essentially of extracellular vesicles (EVs) from Prevotella histicola bacteria and an excipient that comprises a lyoprotectant.

In some aspects, the disclosure provides a solution comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a solution consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such solution, wherein the composition further comprises a pharmaceutically acceptable excipient. In some embodiments, the pharmaceutically acceptable excipient comprises a glidant, lubricant, and/or diluent.

In some aspects, the disclosure provides a dried form comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a dried form consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such dried form, wherein the composition further comprises a pharmaceutically acceptable excipient. In some embodiments, the pharmaceutically acceptable excipient comprises a glidant, lubricant, and/or diluent.

In some aspects, the disclosure provides a powder comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a powder consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such powder, wherein the composition further comprises a pharmaceutically acceptable excipient. In some embodiments, the pharmaceutically acceptable excipient comprises a glidant, lubricant, and/or diluent.

In some aspects, the disclosure provides a spray-dried powder comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a spray-dried powder consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such spray-dried powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilate comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a lyophilate consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such lyophilate, wherein the composition further comprises a pharmaceutically acceptable excipient. In some embodiments, the pharmaceutically acceptable excipient comprises a glidant, lubricant, and/or diluent.

In some aspects, the disclosure provides a lyophilized powder comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a lyophilized powder consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such lyophilized powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilized cake comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a lyophilized cake consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such lyophilized cake, wherein the composition further comprises a pharmaceutically acceptable excipient. In some embodiments, the pharmaceutically acceptable excipient comprises a glidant, lubricant, and/or diluent.

In some aspects, the disclosure provides a method of treating a subject (for example, human) (for example, a subject in need of treatment), the method comprising: administering to the subject Prevotella histicola EVs or a solution, dried form, or therapeutic composition described herein.

In some embodiments, Prevotella histicola EVs or a solution, dried form, or therapeutic composition provided herein is for use in treating a subject (for example, human) (for example, a subject in need of treatment), as described herein.

In some aspects, the disclosure provides use of Prevotella histicola EVs or a solution, dried form, or therapeutic composition provided herein for the preparation of a medicament for treating a subject (for example, human) (for example, a subject in need of treatment), as described herein.

In some embodiments of the method, solution, dried form, therapeutic composition or use provided herein, the Prevotella histicola EVs or solution, dried form, or therapeutic composition is orally administered (for example, is for oral administration).

In some embodiments of the method, solution, dried form, therapeutic composition or use provided herein, the solution, dried form, or therapeutic composition is administered in combination with an additional therapeutic agent.

In some embodiments of the method, solution, dried form, therapeutic composition or use provided herein, the solution, dried form, or therapeutic composition is administered in combination with an additional therapy.

In some embodiments of the method, solution, dried form, therapeutic composition or use provided herein, the dried form is a powder. In some embodiments, the powder is a lyophilized powder. In some embodiments, the powder is a spray-dried powder.

In some embodiments of the method, solution, dried form, therapeutic composition or use provided herein, the dried form is a lyophilate. In some embodiments, the lyophilate is a lyophilized powder. In some embodiments, the lyophilate is a lyophilized cake.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent, thereby preparing the solution.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant, thereby preparing the solution.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant, thereby preparing the solution.

In some embodiments, the disclosure provides a solution prepared by a method described herein.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
drying the solution to prepare a cake, and
milling (for example, grinding) the cake, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
drying the solution to prepare a cake, and
milling (for example, grinding) the cake, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) lyoprotectant to prepare a solution;
drying the solution to prepare a cake, and
milling (for example, grinding) the cake, thereby preparing the dried form.

In some embodiments of the methods of preparing a dried form provided herein, the drying comprises lyophilization.

In some embodiments of the methods of preparing a dried form provided herein, the drying comprises spray drying.

In some embodiments of the methods of preparing a dried form provided herein, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
drying the solution to prepare a cake, and
milling (for example, grinding) the cake, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
drying the solution to prepare a cake, and
milling (for example, grinding) the cake, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
drying the solution to prepare a cake, and
milling (for example, grinding) the cake, thereby preparing the powder.

In some embodiments of the methods of preparing a powder provided herein, the drying comprises lyophilization.

In some embodiments of the methods of preparing a powder provided herein, the drying comprises spray drying.

In some embodiments of the methods of preparing a powder provided herein, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
spray drying the solution, thereby preparing the spray-dried powder.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
spray drying the solution, thereby preparing the spray-dried powder.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
spray drying the solution, thereby preparing the spray-dried powder.

In some embodiments of the methods of preparing a spray-dried powder provided herein, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
freeze drying (lyophilizing) the solution to prepare a cake, and
milling (for example, grinding) the cake, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
freeze drying (lyophilizing) the solution to prepare a cake, and
milling (for example, grinding) the cake, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
freeze drying (lyophilizing) the solution to prepare a cake, and
milling (for example, grinding) the cake, thereby preparing the lyophilate.

In some embodiments of the methods of preparing a lyophilate provided herein, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
freeze drying (lyophilizing) the solution to prepare a cake, and
milling (for example, grinding) the cake, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
freeze drying (lyophilizing) the solution to prepare a cake, and
milling (for example, grinding) the cake, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
freeze drying (lyophilizing) the solution to prepare a cake, and
milling (for example, grinding) the cake, thereby preparing the lyophilized powder.

In some embodiments of the methods of preparing a lyophilized powder provided herein, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilized cake.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilized cake.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilized cake.

In some embodiments, the disclosure provides a lyophilized cake prepared by a method described herein.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution.

In some embodiments, the disclosure provides a solution prepared by a method described herein.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
drying the solution to prepare a cake, and
milling (for example, grinding) the cake, thereby preparing the dried form.

In some embodiments of the methods of preparing a dried form provided herein, the drying comprises lyophilization.

In some embodiments of the methods of preparing a dried form provided herein, the drying comprises spray drying.

In some embodiments of the methods of preparing a dried form provided herein, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
drying the solution to prepare a cake, and
milling (for example, grinding) the cake, thereby preparing the powder.

In some embodiments of the methods of preparing a powder provided herein, the drying comprises lyophilization.

In some embodiments of the methods of preparing a powder provided herein, the drying comprises spray drying.

In some embodiments of the methods of preparing a powder provided herein, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
spray drying the solution, thereby preparing the spray-dried powder.

In some embodiments of the methods of preparing a spray-dried powder provided herein, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
freeze drying (lyophilizing) the solution to prepare a cake, and
milling (for example, grinding) the cake, thereby preparing the lyophilate.

In some embodiments of the methods of preparing a lyophilate provided herein, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
freeze drying (lyophilizing) the solution to prepare a cake, and
milling (for example, grinding) the cake, thereby preparing the lyophilized powder.

In some embodiments of the methods of preparing a lyophilized powder provided herein, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing a lyophilized cake.

In some embodiments, the disclosure provides a lyophilized cake prepared by a method described herein.

In some embodiments, the disclosure provides a lyophilized cake prepared by a method described herein.

In some embodiments of the methods that comprise a freeze drying step, the freeze drying comprises primary drying and secondary drying. In some embodiments, primary drying is performed at a temperature between about −35° C. to about −20° C. For example, primary drying is performed at a temperature of about −20° C., about −25° C., about −30° C. or about −35° C. In some embodiments, secondary drying is performed at a temperature between about +20° C. to about +30° C. For example, secondary drying is performed at a temperature of about +25° C.

In some embodiments of the solution, dried form, or therapeutic composition provided herein, the bulking agent comprises mannitol, sucrose, maltodextrin, dextran, Ficoll, polyethylene glycol (PEG, such as PEG 6000), cyclodextrin, or PVP-K30.

In some embodiments of the solution, dried form, or therapeutic composition provided herein, the bulking agent comprises mannitol.

In some embodiments of the solution, dried form, or therapeutic composition provided herein, the excipient comprises an additional ingredient.

In some embodiments of the solution, dried form, or therapeutic composition provided herein, the additional ingredient comprises trehalose, mannitol, sucrose, sorbitol, dextran, poloxamer 188, maltodextrin, PVP-K30, Ficoll, citrate, arginine, and/or hydroxypropyl-β-cyclodextrin.

In some embodiments of the solution, dried form, or therapeutic composition provided herein, the excipient comprises mannitol and trehalose.

In some embodiments of the solution, dried form, or therapeutic composition provided herein, the excipient consists essentially of mannitol and trehalose.

In some embodiments of the solution, dried form, or therapeutic composition provided herein, the excipient comprises mannitol, trehalose, and sorbitol.

In some embodiments of the solution, dried form, or therapeutic composition provided herein, the excipient consists essentially of mannitol, trehalose, and sorbitol.

In some embodiments of the solution, dried form, or therapeutic composition provided herein, the excipient comprises trehalose.

In some embodiments of the solution, dried form, or therapeutic composition provided herein, the excipient consists essentially of trehalose.

In some embodiments of the solution, dried form, or therapeutic composition provided herein, the excipient is from a stock comprising one or more excipients, wherein the stock comprises a formula provided in provided in Table A, B, C, D, K, or P.

In some embodiments of the solution, dried form, or therapeutic composition provided herein, the dried form is a powder. In some embodiments, the powder is a lyophilized powder. In some embodiments, the powder is a spray-dried powder.

In some embodiments of the solution, dried form, or therapeutic composition provided herein, the dried form is a lyophilate. In some embodiments, the lyophilate is a lyophilized powder. In some embodiments, the lyophilate is a lyophilized cake.

In some embodiments of the solution, dried form, or therapeutic composition provided herein, the excipient solution comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient solution comprises more mannitol than trehalose, for example, on a weight basis or weight percent basis. In some embodiments, the excipient solution comprises at least two-fold more mannitol than trehalose, for example, on a weight basis or weight percent basis. In some embodiments, the excipient solution comprises at least three-fold more mannitol than trehalose, for example, on a weight basis or weight percent basis. In some embodiments, the excipient of the solution or dried form comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient of the solution or dried form comprises more mannitol than trehalose, for example, on a weight basis or weight percent basis. In some embodiments, the excipient of the solution or dried form comprises at least two-fold more mannitol than trehalose, for example, on a weight basis or weight percent basis. In some embodiments, the excipient of the solution or dried form comprises at least three-fold more mannitol than trehalose, for example, on a weight basis or weight percent basis.

In some embodiments of the solution, dried form, or therapeutic composition provided herein, the excipient solution consists essentially of mannitol and trehalose. In some embodiments, the excipient solution consists essentially of mannitol and trehalose, wherein the mannitol and the trehalose are not present in equal amounts (for example, the mannitol and the trehalose are present in unequal amounts; for example, on a weight basis or a weight percent basis). In some embodiments, the excipient solution consists essentially of mannitol and trehalose, wherein the excipient contains more mannitol than trehalose, for example, on a weight basis or weight percent basis. In some embodiments, the excipient solution consists essentially of mannitol and trehalose, wherein the excipient solution contains at least two-fold more mannitol than trehalose, for example, on a weight basis or weight percent basis. In some embodiments, the excipient solution consists essentially of mannitol and trehalose, wherein the excipient solution contains at least three-fold more mannitol than trehalose, for example, on a weight basis or weight percent basis. In some embodiments, the excipient of the solution or dried form consists essentially of mannitol and trehalose, wherein the excipient of the solution or dried form contains more mannitol than trehalose, for example, on a weight basis or weight percent basis. In some embodiments, the excipient of the solution or dried form consists essentially of mannitol and trehalose, wherein the excipient of the solution or dried form contains at least two-fold more mannitol than trehalose, for example, on a weight basis or weight percent basis. In some embodiments, the excipient of the solution or dried form consists essentially of mannitol and trehalose, wherein the excipient of the solution or dried form contains at least three-fold more mannitol than trehalose, for example, on a weight basis or weight percent basis.

In some embodiments of the solution, dried form, or therapeutic composition provided herein, the excipient solution comprises, or consists essentially of, mannitol and trehalose, wherein neither the mannitol nor the trehalose is present in an amount of 5 mg/ml to 15 mg/ml. In some embodiments, the excipient solution comprises, or consists essentially of, mannitol and trehalose, wherein the mannitol is not present in an amount of 5 mg/ml to 15 mg/ml. In some embodiments, the excipient solution comprises, or consists essentially of, mannitol and trehalose, wherein the trehalose is not present in an amount of 5 mg/ml to 15 mg/ml.

In some embodiments of the solution, dried form, or therapeutic composition provided herein, the excipient solution comprises, or consists essentially of, mannitol and trehalose, wherein neither the mannitol nor the trehalose is present in an amount of 9 mg/ml. In some embodiments, the excipient solution comprises, or consists essentially of, mannitol and trehalose, wherein the mannitol is not present in an amount of 9 mg/ml. In some embodiments, the excipient solution comprises, or consists essentially of, mannitol and trehalose, wherein the trehalose is not present in an amount of 9 mg/ml.

In some embodiments of the solution, dried form, or therapeutic composition provided herein, the excipient comprises, or consists essentially of, mannitol and trehalose, and does not comprise methionine.

In some embodiments of the dried form or therapeutic composition provided herein, the dried form or therapeutic composition comprises, or consists essentially of, mannitol and trehalose, and the mannitol and the trehalose are not present in equal amounts (for example, the mannitol and the trehalose are present in unequal amounts, for example, on a weight basis or a weight percent basis) in the dried form or therapeutic composition.

In some embodiments of the solution, dried form, or therapeutic composition provided herein, at least about 10% (by weight) of the solution or dried form is excipient stock.

In some embodiments of the solution, dried form, or therapeutic composition provided herein, about 10% to about 80% (by weight) of the solution or dried form is excipient stock.

In some embodiments of the solution, dried form, or therapeutic composition provided herein, wherein about 20% to about 70% (by weight) of the solution or dried form is excipient stock.

In some embodiments of the solution, dried form, or therapeutic composition provided herein, about 30% to about 60% (by weight) of the solution or dried form is excipient stock.

In some embodiments of the dried form or therapeutic composition provided herein, the EVs from Prevotella histicola bacteria comprise at least about 1% of the total solids by weight of the dried form.

In some embodiments of the dried form or therapeutic composition provided herein, the EVs from Prevotella histicola bacteria comprise about 1% to about 99% of the total solids by weight of the dried form.

In some embodiments of the dried form or therapeutic composition provided herein, the EVs from Prevotella histicola bacteria comprise about 5% to about 90% of the total solids by weight of the dried form. In some embodiments of the dried form or therapeutic composition provided herein, the EVs from Prevotella histicola bacteria comprise about 10% to about 60% of the total solids by weight of the dried form. In some embodiments of the dried form or therapeutic composition provided herein, the EVs from Prevotella histicola bacteria comprise about 1% to about 20% of the total solids by weight of the powder or cake. In some embodiments of the dried form or therapeutic composition provided herein, the EVs from Prevotella histicola bacteria comprise about 2% to about 10% of the total solids by weight of the dried form. In some embodiments of the dried form or therapeutic composition provided herein, the EVs from Prevotella histicola bacteria comprise about 2% to about 6% of the total solids by weight of the dried form. In some embodiments of the dried form or therapeutic composition provided herein, the dried form comprises a moisture content below about 6% (for example, as determined by Karl Fischer titration).

In some embodiments of the dried form or therapeutic composition provided herein, the dried form comprises a moisture content below about 5% (for example, as determined by Karl Fischer titration).

In some embodiments of the dried form or therapeutic composition provided herein, the dried form comprises a moisture content about 0.5% to about 5% (for example, as determined by Karl Fischer titration).

In some embodiments of the dried form or therapeutic composition provided herein, the dried form comprises a moisture content about 1% to about 5% (for example, as determined by Karl Fischer titration).

In some embodiments of the dried form or therapeutic composition provided herein, the dried form comprises a moisture content about 1% to about 4% (for example, as determined by Karl Fischer titration).

In some embodiments of the dried form or therapeutic composition provided herein, the dried form comprises a moisture content about 2% to about 5% (for example, as determined by Karl Fischer titration).

In some embodiments of the dried form or therapeutic composition provided herein, the dried form comprises a moisture content about 2% to about 4% (for example, as determined by Karl Fischer titration).

In some embodiments of the dried form or therapeutic composition provided herein, the dried form comprises at least 1 e10 particles per mg of the dried form (for example, as determined by particles per mg, such as by NTA).

In some embodiments of the dried form or therapeutic composition provided herein, the dried form comprises about 3 e10 to about 6.5 e10 particles per mg of the dried form (for example, as determined by particles per mg, such as by NTA).

In some embodiments of the dried form or therapeutic composition provided herein, the dried form comprises about 3 e10 to about 8 e10 particles per mg of the dried form (for example, as determined by particles per mg, such as by NTA).

In some embodiments of the dried form or therapeutic composition provided herein, the dried form comprises about 6 e10 to about 8 e10 particles per mg of the dried form (for example, as determined by particles per mg, such as by NTA).

In some embodiments of the dried form or therapeutic composition provided herein, the dried form comprises about 6.7 e8 to about 2.55 e10 particles/mg dried form (for example, as determined by particles per mg, such as by NTA).

In some embodiments of the dried form or therapeutic composition provided herein, the dried form comprises about 6.7 e8 to about 2.89 e10 particles/mg dried form.

In some embodiments, particle numeration is determined on a dried form by NTA. In some embodiments, particle numeration is determined on a dried form by NTA with use of a Zetaview camera.

In some embodiments, particle numeration is determined on dried form resuspended in water, by NTA and with use of a Zetaview camera.

In some embodiments of the dried form or therapeutic composition provided herein, the particles have a hydrodynamic diameter (Z average, Zₐᵥₑ) of about 100 nm to about 300 nm after resuspension from the dried form (for example, resuspension in deionized water) (for example, as determined by dynamic light scattering).

In some embodiments of the dried form or therapeutic composition provided herein, the particles have a hydrodynamic diameter (Z average, Zₐᵥₑ) of about 130 nm to about 250 nm after resuspension from the dried form (for example, resuspension in deionized water) (for example, as determined by dynamic light scattering).

In some embodiments of the dried form or therapeutic composition provided herein, the particles have a hydrodynamic diameter (Z average, Zₐᵥₑ) of about 200 nm after resuspension from the dried form (for example, resuspension in deionized water) (for example, as determined by dynamic light scattering).

In some embodiments, a solution, dried form, or therapeutic composition provided herein can contain EVs from one or more bacterial strain in addition to EVs from Prevotella histicola. In some embodiments, a solution, dried form, or therapeutic composition provided herein can contain EVs from one bacterial strain in addition to EVs from Prevotella histicola. The bacterial strain used as a source of EVs may be selected based on the properties of the bacteria (e.g., growth characteristics, yield, ability to modulate an immune response in an assay or a subject).

In some embodiments, Prevotella histicola EVs or a solution, dried form, or therapeutic composition provided herein comprising EVs from Prevotella histicola bacteria can be used for the treatment or prevention of a disease and/or a health disorder, e.g., in a subject (e.g., human).

In some embodiments, a dried form (or a therapeutic composition thereof) provided herein comprising EVs from Prevotella histicola bacteria can be prepared as a solid dose form, such as a tablet, a minitablet, a capsule, or a powder; or a combination of these forms (e.g., minitablets comprised in a capsule). The solid dose form can comprise a coating (e.g., enteric coating).

In certain embodiments, the therapeutic composition comprises a solid dose form. In some embodiments, the therapeutic composition comprises a blend of freeze-dried powder of EVs from Prevotella histicola bacteria and excipients (e.g., an encapsulated freeze-dried powder of the EVs from Prevotella histicola bacteria provided herein and excipients). In some embodiments, the therapeutic composition comprises freeze-dried (e.g., lyophilized) powder of EVs from Prevotella histicola bacteria in a capsule. In some embodiments, the capsule comprises gelatin or hydroxyl propyl methyl cellulose HPMC. In some embodiments, the capsule is enteric coated. In some embodiments, the excipients include one or more of mannitol, magnesium stearate and colloidal silicon dioxide. In some embodiments, the excipients include mannitol, magnesium stearate and colloidal silicon dioxide. In some embodiments, the therapeutic composition comprises freeze-dried (e.g., lyophilized) powder of EVs from Prevotella histicola bacteria in a tablet or mini-tablet. In some embodiments, the tablet or mini-tablet is enteric coated. In some embodiments, the excipients include one or more of silicified microcrystalline cellulose, crospovidone, magnesium stearate and colloidal silicon dioxide. In some embodiments, the excipients include silicified microcrystalline cellulose, crospovidone, magnesium stearate and colloidal silicon dioxide.

In some embodiments, a dried form (or a therapeutic composition thereof) provided herein comprising EVs from Prevotella histicola bacteria can be reconstituted. In some embodiments, a solution (or a therapeutic composition thereof) provided herein comprising EVs from Prevotella histicola bacteria can be used as suspension, e.g., diluted to a suspension or used in undiluted form.

In some embodiments, a therapeutic composition comprising Prevotella histicola EVs or a solution and/or dried form comprising EVs from Prevotella histicola bacteria can be prepared as provided herein. The therapeutic composition comprising a dried form can be formulated into a solid dose form, such as a tablet, a minitablet, a capsule, or a powder; or can be reconstituted in a suspension.

In some embodiments, Prevotella histicola EVs or a solution, dried form, or therapeutic composition provided herein can comprise gamma irradiated EVs from Prevotella histicola bacteria. The gamma irradiated EVs from Prevotella histicola bacteria can be formulated into a therapeutic composition. The gamma irradiated EVs from Prevotella histicola bacteria can be formulated into a solid dose form, such as a tablet, a minitablet, a capsule, or a powder; or can be reconstituted in a suspension.

In some embodiments, Prevotella histicola EVs or a solution, dried form, or therapeutic composition provided herein comprising EVs from Prevotella histicola bacteria can be orally administered.

In some embodiments, Prevotella histicola EVs or a solution, dried form, or therapeutic composition provided herein comprising EVs from Prevotella histicola bacteria can be administered intranasally.

In some embodiments, Prevotella histicola EVs or a solution, dried form, or therapeutic composition provided herein comprising EVs from Prevotella histicola bacteria can be administered by inhalation.

In some embodiments, Prevotella histicola EVs or a solution, dried form, or therapeutic composition provided herein comprising EVs from Prevotella histicola bacteria can be administered intravenously.

In some embodiments, Prevotella histicola EVs or a solution, dried form, or therapeutic composition provided herein comprising EVs from Prevotella histicola bacteria can be administered by injection.

In some embodiments, the therapeutic compositions can comprise both EVs from Prevotella histicola bacteria and whole bacteria, e.g., Prevotella histicola bacteria from which the EVs were obtained, such as live bacteria, killed bacteria, attenuated bacteria. In some embodiments, the therapeutic compositions comprise EVs from Prevotella histicola bacteria in the absence of the bacteria from which they were obtained, such that over about 85%, over about 90%, or over about 95% (or over about 99%) of the bacteria-sourced content of the solutions and/or dried forms comprises Prevotella histicola EVs. The Prevotella histicola EVs can be isolated EVs, e.g., isolated by a method described herein.

In some embodiments, the Prevotella histicola EVs or solution, dried form, or therapeutic composition comprises isolated Prevotella histicola EVs (e.g., from one or more strains of bacteria (e.g., a therapeutically effective amount thereof). E.g., wherein at least 50%, at least 75%, at least 80%, at least 85%, at least 90%, at least 95%, or at least 99% of the content (e.g., of the content that does not exclude excipient) of the Prevotella histicola EVs and/or solution and/or dried form is isolated EVs from Prevotella histicola bacteria (e.g., bacteria of interest).

In some embodiments, the Prevotella histicola EVs or solution, dried form, or therapeutic composition comprises isolated Prevotella histicola EVs (e.g., from one strain of bacteria (e.g., bacteria of interest) (e.g., a therapeutically effective amount thereof). E.g., wherein at least 50%, at least 75%, at least 80%, at least 85%, at least 90%, at least 95%, or at least 99% of the content (e.g., of the content that does not exclude excipient) of the Prevotella histicola EVs and/or solution and/or dried form is isolated EV of Prevotella histicola bacteria (e.g., bacteria of interest, e.g., bacteria disclosed herein).

In some embodiments, the Prevotella histicola EVs or solution, dried form or therapeutic composition comprises EVs from Prevotella histicola bacteria.

In some embodiments, the solution, dried form, or therapeutic composition comprises EVs from more than one strain of bacteria (e.g., EVs from a strain in addition to the Prevotella histicola EVs).

In some embodiments, the Prevotella histicola EVs are lyophilized.

In some embodiments, the Prevotella histicola EVs are gamma irradiated.

In some embodiments, the Prevotella histicola EVs are UV irradiated.

In some embodiments, the Prevotella histicola EVs are heat inactivated (e.g., at 50° C. for two hours or at 90° C. for two hours).

In some embodiments, the Prevotella histicola EVs are acid treated.

In some embodiments, the Prevotella histicola EVs are oxygen sparged (e.g., at 0.1 vvm for two hours).

In some embodiments, the Prevotella histicola EVs are from a strain comprising at least 90% (or at least 97%) genomic, 16S and/or CRISPR sequence identity to the nucleotide sequence of the Prevotella Strain B 50329 (NRRL accession number B 50329). In some embodiments, the Prevotella histicola EVs are from a strain comprising at least 99% genomic, 16S and/or CRISPR sequence identity to the nucleotide sequence of the Prevotella Strain B 50329 (NRRL accession number B 50329). In some embodiments, the Prevotella histicola bacteria are from Prevotella Strain B 50329 (NRRL accession number B 50329).

In certain aspects, the Prevotella histicola EVs obtained from bacteria that have been selected based on certain desirable properties, such as reduced toxicity and adverse effects (e.g., by removing or deleting lipopolysaccharide (LPS)), enhanced oral delivery (e.g., by improving acid resistance, muco-adherence and/or penetration and/or resistance to bile acids, resistance to anti-bacterial peptides and/or antibody neutralization), target desired cell types (e.g., M-cells, goblet cells, enterocytes, dendritic cells, macrophages), improved bioavailability systemically or in an appropriate niche (e.g., mesenteric lymph nodes, Peyer's patches, lamina propria, lymph nodes, and/or blood), enhanced immunomodulatory and/or therapeutic effect (e.g., either alone or in combination with another therapeutic agent), enhanced immune activation, and/or manufacturing attributes (e.g., growth characteristics, yield, greater stability, improved freeze-thaw tolerance, shorter generation times).

In certain aspects, the Prevotella histicola EVs are from engineered bacteria that are modified to enhance certain desirable properties. In some embodiments, the engineered bacteria are modified so that EVs produced therefrom will have reduced toxicity and adverse effects (e.g., by removing or deleting lipopolysaccharide (LPS)), enhanced oral delivery (e.g., by improving acid resistance, muco-adherence and/or penetration and/or resistance to bile acids, resistance to anti-microbial peptides and/or antibody neutralization), target desired cell types (e.g., M-cells, goblet cells, enterocytes, dendritic cells, macrophages), improved bioavailability systemically or in an appropriate niche (e.g., mesenteric lymph nodes, Peyer's patches, lamina propria, lymph nodes, and/or blood), enhanced immunomodulatory and/or therapeutic effect (e.g., either alone or in combination with another therapeutic agent), enhanced immune activation, and/or improved manufacturing attributes (e.g., growth characteristics, yield, greater stability, improved freeze-thaw tolerance, shorter generation times). In some embodiments, provided herein are methods of making such EVs.

In certain aspects, provided herein are Prevotella histicola EVs and/or solutions and/or dried forms (or therapeutic compositions thereof) comprising EVs from Prevotella histicola bacteria useful for the treatment and/or prevention of a disease or a health disorder, as well as methods of making and/or identifying such solutions and/or dried forms (or therapeutic compositions thereof), and methods of using such solutions and/or dried forms (e.g., for the treatment of a diseae or health disorder), either alone or in combination with one or more other therapeutics.

Therapeutic compositions containing Prevotella histicola EVs and/or a solution and/or dried form can provide potency comparable to or greater than therapeutic compositions that contain the whole Prevotella histicola bacteria from which the EVs were obtained. For example, at the same dose of EVs (e.g., based on particle count or protein content), a therapeutic composition containing solutions and/or dried form can provide potency comparable to or greater than a comparable therapeutic composition that contains whole bacteria of the same Prevotella histicola bacterial strain from which the EVs were obtained. Such EV—and/or solution—and/or dried form—containing therapeutic compositions can allow the administration of higher doses and elicit a comparable or greater (e.g., more effective) response than observed with a comparable therapeutic composition that contains whole bacteria of the same Prevotella histicola bacterial strain from which the EVs were obtained.

As a further example, at the same dose (e.g., based on particle count or protein content), a therapeutic composition containing Prevotella histicola EVs and/or a solution and/or dried form can contain less microbially-derived material (based on particle count or protein content), as compared to a therapeutic composition that contains the whole Prevotella histicola bacteria of the same bacterial strain from which the EVs were obtained, while providing an equivalent or greater therapeutic benefit to the subject receiving such therapeutic composition.

As a further example, EVs from Prevotella histicola bacteria can be administered at doses e.g., of about 1×10⁷ to about 1×10¹⁵ particles, e.g., as measured by NTA. In some embodiments, the dose of EVs is about 1×10⁵ to about 7×10¹³ particles (e.g., wherein particle count is determined by NTA (nanoparticle tracking analysis)). In some embodiments, the dose of EVs from Prevotella histicola bacteria is about 1×10¹⁰ to about 7×10¹³ particles (e.g., wherein particle count is determined by NTA (nanoparticle tracking analysis)). NTA can be performed with Zetaview.

As another example, EVs from Prevotella histicola bacteria can be administered at doses e.g., of about 5 mg to about 900 mg total protein, e.g., as measured by Bradford assay. As another example, EVs from Prevotella histicola bacteria can be administered at doses e.g., of about 5 mg to about 900 mg total protein, e.g., as measured by BCA assay.

A therapeutic composition or Prevotella histicola EVs and/or a solution and/or dried form, e.g., as described herein, comprising EVs from Prevotella histicola bacteria can provide a therapeutically effective amount of Prevotella histicola EVs to a subject, e.g., a human.

A therapeutic composition or Prevotella histicola EVs and/or a solution and/or dried form, e.g., as described herein, comprising EVs from Prevotella histicola bacteria can provide a non-natural amount of the therapeutically effective components (e.g., present in the Prevotella histicola EVs to a subject, e.g., a human.

A therapeutic composition or Prevotella histicola EVs and/or a solution and/or dried form, e.g., as described herein, comprising EVs from Prevotella histicola bacteria can provide unnatural quantity of the therapeutically effective components (e.g., present in the EVs to a subject, e.g., a human.

A therapeutic composition or Prevotella histicola EVs and/or a solution and/or dried form, e.g., as described herein, comprising EVs from Prevotella histicola bacteria can bring about one or more changes to a subject, e.g., human, e.g., to treat or prevent a disease or a health disorder.

A therapeutic composition or Prevotella histicola EVs and/or a solution and/or dried form, e.g., as described herein, comprising EVs from Prevotella histicola bacteria has potential for significant utility, e.g., to affect a subject, e.g., a human, e.g., to treat or prevent a disease or a health disorder.

In certain aspects, provided herein is a stock comprising one or more excipients, wherein the stock comprises a bulking agent, wherein the stock is for use in combination with extracellular vesicles (EVs) from Prevotella histicola bacteria (for example, a liquid preparation thereof).

In certain aspects, provided herein is a stock comprising one or more excipients, wherein the stock comprises a bulking agent and a lyoprotectant, wherein the stock is for use in combination with extracellular vesicles (EVs) from Prevotella histicola bacteria (for example, a liquid preparation thereof).

In certain aspects, provided herein is a stock comprising one or more excipients, wherein the stock comprises a lyoprotectant, wherein the stock is for use in combination with extracellular vesicles (EVs) from Prevotella histicola bacteria (for example, a liquid preparation thereof).

In some embodiments, the bulking agent comprises mannitol, sucrose, maltodextrin, dextran, Ficoll, or PVP-K30.

In some embodiments, the bulking agent comprises mannitol.

In some embodiments, the excipient solution comprises an additional ingredient.

In some embodiments, the additional ingredient comprises trehalose, mannitol, sucrose, sorbitol, dextran, poloxamer 188, maltodextrin, PVP-K30, Ficoll, citrate, arginine, and/or hydroxypropyl-β-cyclodextrin.

In some embodiments, the excipient solution comprises mannitol and trehalose.

In some embodiments, the excipient solution consists essentially of mannitol and trehalose.

In some embodiments, the excipient solution comprises mannitol, trehalose, and sorbitol.

In some embodiments, the excipient solution consists essentially of mannitol, trehalose, and sorbitol.

In some embodiments, the excipient solution comprises trehalose.

In some embodiments, the excipient solution consists essentially of trehalose.

In some embodiments, the excipient solution comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient solution comprises more mannitol than trehalose, for example, on a weight basis or weight percent basis. In some embodiments, the excipient solution comprises at least two-fold more mannitol than trehalose, for example, on a weight basis or weight percent basis. In some embodiments, the excipient solution comprises at least three-fold more mannitol than trehalose, for example, on a weight basis or weight percent basis. In some embodiments, the excipient of the solution or dried form comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient of the solution or dried form comprises more mannitol than trehalose, for example, on a weight basis or weight percent basis. In some embodiments, the excipient of the solution or dried form comprises at least two-fold more mannitol than trehalose, for example, on a weight basis or weight percent basis. In some embodiments, the excipient of the solution or dried form comprises at least three-fold more mannitol than trehalose, for example, on a weight basis or weight percent basis.

In some embodiments, the excipient solution consists essentially of mannitol and trehalose. In some embodiments, the excipient solution consists essentially of mannitol and trehalose, wherein the mannitol and the trehalose are not present in equal amounts (for example, the mannitol and the trehalose are present in unequal amounts; for example, on a weight basis or a weight percent basis). In some embodiments, the excipient solution consists essentially of mannitol and trehalose, wherein the excipient solution contains more mannitol than trehalose, for example, on a weight basis or weight percent basis. In some embodiments, the excipient solution consists essentially of mannitol and trehalose, wherein the excipient solution contains at least two-fold more mannitol than trehalose, for example, on a weight basis or weight percent basis. In some embodiments, the excipient solution consists essentially of mannitol and trehalose, wherein the excipient solution contains at least three-fold more mannitol than trehalose, for example, on a weight basis or weight percent basis. In some embodiments, the excipient of the solution or dried form consists essentially of mannitol and trehalose, wherein the excipient of the solution or dried form contains more mannitol than trehalose, for example, on a weight basis or weight percent basis. In some embodiments, the excipient of the solution or dried form consists essentially of mannitol and trehalose, wherein the excipient of the solution or dried form contains at least two-fold more mannitol than trehalose, for example, on a weight basis or weight percent basis. In some embodiments, the excipient of the solution or dried form consists essentially of mannitol and trehalose, wherein the excipient of the solution or dried form contains at least three-fold more mannitol than trehalose, for example, on a weight basis or weight percent basis.

In some embodiments, the excipient solution comprises, or consists essentially of, mannitol and trehalose, wherein neither the mannitol nor the trehalose is present in an amount of 5 mg/ml to 15 mg/ml. In some embodiments, the excipient solution comprises, or consists essentially of, mannitol and trehalose, wherein the mannitol is not present in an amount of 5 mg/ml to 15 mg/ml. In some embodiments, the excipient solution comprises, or consists essentially of, mannitol and trehalose, wherein the trehalose is not present in an amount of 5 mg/ml to 15 mg/ml.

In some embodiments, the excipient solution comprises, or consists essentially of, mannitol and trehalose, wherein neither the mannitol nor the trehalose is present in an amount of 9 mg/ml. In some embodiments, the excipient solution comprises, or consists essentially of, mannitol and trehalose, wherein the mannitol is not present in an amount of 9 mg/ml. In some embodiments, the excipient solution comprises, or consists essentially of, mannitol and trehalose, wherein the trehalose is not present in an amount of 9 mg/ml.

In some embodiments, the excipient solution comprises, or consists essentially of, mannitol and trehalose, and does not comprise methionine.

In certain aspects, provided herein is a stock comprising one or more excipients, wherein the stock comprises a formula provided in Table A, B, C, D, K, or P.

In certain aspects, provided herein is a stock comprising one or more excipients, wherein the stock comprises a formula provided in Table A, B, C, D, K, or P wherein the stock is for use in combination with extracellular vesicles (EVs) from Prevotella histicola bacteria (for example, a liquid preparation thereof).

In some embodiments of the solutions and dried forms and methods described herein, a liquid preparation comprises a cell culture supernatant, such as a bacterial cell culture supernatant, for example, as described herein. In some embodiments of the solution and dried forms and methods described herein, the liquid preparation comprises a retentate, such as a concentrated retentate, for example, as described herein.

In some embodiments of the methods provided herein, excipients are present in (for example, provided in) an excipient solution. Examples of an excipient solution include the stocks comprising one or more excipients provided in Tables A, B, C, D, K, or P. For example, the dried forms provided herein contain excipients from the excipient solution (such as a stock) once the moisture has been removed, such as by drying. For example, a liquid preparation that comprises EVs from Prevotella histicola bacteria is combined with the stock of formula 7a (which comprises the excipients mannitol and trehalose) from Table A to prepare a solution. The solution is dried to prepare a dried form. The dried form comprises EVs from Prevotella histicola bacteria, mannitol, and trehalose. As used herein, a “stock” refers to a solution comprising one or more excipients but no active ingredient (such as an extracellular vesicle). In some embodiments, a stock is used to introduce one or more excipients into a preparation (such as a liquid preparation) comprising EVs. In some embodiments, the stock is a concentrated solution comprising a known amount of one or more excipients. In some embodiments, the stock is combined with a preparation (such as a liquid preparation) that comprises EVs to prepare a solution or dried form provided herein.

In certain embodiments, the therapeutic composition is provided as a solid dosage form (also referred to as a solid dose form). In some embodiments, provided herein are solid dosage forms comprising extracellular vesicles (EVs) from Prevotella bacteria. In some embodiments, the solid dosage form comprises an enteric coating (e.g., HPMC coat).

In some embodiments, the solid dosage form comprises a capsule. In some embodiments, the capsule is an enteric coated capsule. In some embodiments, the enteric coating comprises HPMC. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P).

In some embodiments, 1, 2, 3, 4, 5, 6, 7, 8, 9, or 10 capsules are administered, e.g., once or twice daily to a subject.

In some embodiments, the Prevotella histicola EVs in the capsule are in dried form (e.g., in a powder). In some embodiments, the extracellular vesicles from Prevotella bacteria in the capsule are lyophilized (e.g., in a freeze-dried powder). In some embodiments, the extracellular vesicles from Prevotella bacteria in the capsule are spray dried (e.g., in a spray-dried powder). In some embodiments, the Prevotella histicola EVs in the capsule are in a dried form, and the dried form further comprises mannitol, magnesium stearate, and/or colloidal silicon dioxide.

In some embodiments, the solid dosage form comprises a tablet. In some embodiments, the tablet is an enteric coated tablet. In some embodiments, the tablet is from 5 mm to 18 mm in diameter. In some embodiments, the enteric coating comprises HPMC. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P).

In some embodiments, 1, 2, 3, 4, 5, 6, 7, 8, 9, or 10 tablets are administered, e.g., once or twice daily to a subject.

In some embodiments, the extracellular vesicles from Prevotella bacteria in the tablet are in dried form. In some embodiments, the extracellular vesicles from Prevotella bacteria in the tablet are lyophilized (e.g., in a freeze-dried powder). In some embodiments, the extracellular vesicles from Prevotella bacteria in the tablet are spray dried (e.g., in a spray-dried powder). In some embodiments, the extracellular vesicles from Prevotella bacteria in the tablet are lyophilized in a powder, and the powder further comprises mannitol, magnesium stearate, and/or colloidal silicon dioxide.

In some embodiments, the therapeutic composition comprising extracellular vesicles from Prevotella bacteria is prepared as a dried form (e.g., for resuspension or for use in a solid dose form (such as a capsule)) or as a solid dose form, such as a tablet, a mini-tablet, a capsule, a pill, or a powder; or a combination of these forms (e.g., mini-tablets comprised in a capsule). The dried form can comprise a lyophilized or spray-dried powder. In some embodiments, the dried form further comprises mannitol, magnesium stearate, and/or colloidal silicon dioxide.

In some embodiments, 1, 2, 3, 4, 5, 6, 7, 8, 9, or 10 solid dosage forms are administered, e.g., once or twice daily to a subject.

In certain embodiments, provided herein are solid dosage forms comprising the extracellular vesicles from Prevotella bacteria. In some embodiments, the solid dosage form is a tablet, e.g., an enteric coated tablet. In some embodiments, the solid dosage form is a mini-tablet, e.g., an enteric coated mini-tablet. In some embodiments, the solid dosage form is a capsule, e.g., an enteric coated capsule. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P or Eudragit L30-D55).

In some embodiments, the therapeutic composition comprising extracellular vesicles from Prevotella bacteria is prepared as a dried form. In some embodiments, the therapeutic composition comprising extracellular vesicles from Prevotella bacteria is prepared as a powder. The powder can comprise lyophilized extracellular vesicles from Prevotella bacteria. The powder can comprise spray-dried extracellular vesicles from Prevotella bacteria. In some embodiments, the powder further comprises mannitol, magnesium stearate, and/or colloidal silicon dioxide. In some embodiments, the therapeutic composition comprises a dried form comprising extracellular vesicles from Prevotella bacteria. In some embodiments, the dried form comprising extracellular vesicles from Prevotella bacteria (e.g., at a dose provided herein) is resuspended (e.g., in a liquid such as a solution, buffer, water or other beverage, or a food), e.g., for use in the methods provided herein.

In some embodiments, the therapeutic composition is administered orally.

In some embodiments, the administration to the subject once daily. In some embodiments, the therapeutic composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the administration to the subject twice daily. In some embodiments, the therapeutic composition is administered twice daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the extracellular vesicles from Prevotella histicola strain are administered in a therapeutic composition (e.g., a therapeutic composition provided herein). In certain embodiments the therapeutic composition is a solid dose form provided herein. In some embodiments, the therapeutic composition comprises a blend of a dried form of extracellular vesicles from Prevotella histicola and excipients (e.g. an encapsulated dried form of extracellular vesicles from Prevotella histicola strain provided herein and excipients). In some embodiments, the therapeutic composition comprises a dried form of extracellular vesicles from Prevotella histicola in a capsule. In some embodiments, the capsule is enteric coated. In some embodiments, the therapeutic composition comprises an enteric coated hydroxylpropyl methylcellulose (HPMC) hard capsule. In some embodiments, the therapeutic composition comprises a formulation of extracellular vesicles from Prevotella histicola Strain B comprising a dried form of extracellular vesicles from Prevotella histicola and excipients. In some embodiments, the excipients include mannitol, magnesium stearate and colloidal silicon dioxide.

In some embodiments, the therapeutic composition is formulated as multiple enteric-coated mini-tablets of extracellular vesicles from Prevotella histicola drug product filled into capsules. In some embodiments, the therapeutic composition is formulated as multiple enteric-coated mini-tablets of extracellular vesicles from Prevotella histicola drug product filled into capsules, e.g., HPMC capsules (MICs). In some embodiments, the therapeutic composition comprises excipients (e.g., pharmaceutically acceptable excipients). In some embodiments, the therapeutic composition comprises mannitol, colloidal silicon dioxide, hydroxypropyl cellulose, crospovidone, and magnesium stearate.

In some embodiments, the extracellular vesicles from Prevotella histicola strain are from a strain comprising at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the extracellular vesicles are from Prevotella histicola Strain B (NRRL accession number B 50329).

In some aspects, the disclosure provides use of extracellular vesicles from a Prevotella histicola strain provided herein and/or a therapeutic composition (e.g., a therapeutic composition and/or a solid dosage form) described herein (e.g., in an amount described herein) for the preparation of a medicament for the performance of a therapeutic method provided herein. In some aspects, the disclosure provides extracellular vesicles from a Prevotella histicola strain provided herein and/or a therapeutic composition (e.g., a therapeutic composition and/or a solid dosage form) described herein (e.g., in an amount described herein) for use in the performance of a therapeutic method provided herein.

In some embodiments, the subject treated according to the methods provided herein has an IL-8-mediated disease or condition. In certain embodiments, the IL-8 mediated disease or condition comprises Severe Acute Respiratory Syndrome (SARS), influenza, respiratory syncytial viral infection, atherosclerosis, melanoma, ovarian carcinoma, lung cancer, prostate cancer, gastric carcinoma, breast cancer, head-and-neck cancer, colon cancer, colitis-associated cancer, kidney cancer, pancreatic cancer, Crohn's disease (CD), Ulcerative Colitis (UC), Ischemia-Reperfusion injury (IRI), acute lung injury, asthma, chronic obstructive pulmonary disease (COPD), cystic fibrosis (CF), pulmonary fibrosis, multiple sclerosis, psoriasis, atopic dermatitis, rheumatoid arthritis, crescentic glomerulonephritis, IgA nephropathy, membranoproliferative glomerulonephritis, lupus nephritis, or membranous nephropathy, alcoholic hepatitis, or HIV-associated neurocognitive disorder. In certain embodiments, the IL-8 mediated disease or condition comprises Severe Acute Respiratory Syndrome (SARS), influenza, or a respiratory syncytial viral infection. In certain embodiments, the IL-8 mediated disease or condition comprises a coronavirus infection (e.g., MERS, SARS (such as SARS-CoV-2)). In certain embodiments, the IL-8 mediated disease or condition comprises SARS-CoV-2 infection. In certain embodiments, the IL-8 mediated disease or condition is COVID-19.

In some embodiments, the subject treated according to the methods provided herein has an IL-6 mediated disease or condition. In certain embodiments, the IL-6 mediated disease or condition comprises Severe Acute Respiratory Syndrome (SARS), influenza, respiratory syncytial viral infection, Agammaglobulinemia, Amyloidosis, Ankylosing spondylitis, Anti-GBM/Anti-TBM nephritis, Antiphospholipid syndrome, Autoimmune hepatitis, Autoimmune inner ear disease, Atopic dermatitis, Asthma, Castleman disease, Celiac disease, Chagas disease, Chronic recurrent multifocal osteomyelitis, Cogan's syndrome, Cold agglutinin disease, CREST syndrome, Crohn's disease, Dermatomyositis, Devic's disease (neuromyelitis optica), Discoid lupus, Endometriosis, Eosinophilic esophagitis, Eosinophilic fasciitis, Evan's syndrome, Fibromyalgia, Giant cell arteritis, Giant cell myocarditis, Glomerulonephritis, Goodpasture's syndrome, Granulomatosis with polyangiitis, Graves' disease, Guillain-Barre syndrome, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Juvenile arthritis, Juvenile/Type 1 Diabetes, Juvenile myositis, Kawasaki syndrome (Kawasaki Disease (and/or, e.g., Kawasaki disease shock syndrome (KDSS))), Lichen planus, Lichen sclerosis, Lupus (SLE), Meniere's disease, Multiple sclerosis, Myasthenia gravis, Microscopic polyangiitis, Optic neuritis, Pemphigus, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Sjogren's syndrome, Temporal arteritis/Giant cell arteritis, Transverse myelitis, Ulcerative colitis, Uveitis, Vasculitis, Vitiligo, Viral myocarditis, or Wegener's granulomatosis (Granulomatosis with Polyangiitis (GPA)). In certain embodiments, the IL-6 mediated disease or condition comprises Severe Acute Respiratory Syndrome (SARS), influenza, or a respiratory syncytial viral infection. In certain embodiments, the IL-6 mediated disease or condition comprises a coronavirus infection (e.g., MERS, SARS (such as SARS-CoV-2)). In certain embodiments, the IL-6 mediated disease or condition comprises SARS-CoV-2 infection. In certain embodiments, the IL-6 mediated disease or condition is COVID-19.

In some embodiments, the subject treated according to the methods provided herein has an IL-10 mediated disease or condition. In certain embodiments, the IL-10 mediated disease or condition comprises Severe Acute Respiratory Syndrome (SARS), influenza, respiratory syncytial viral infection, Agammaglobulinemia, Amyloidosis, Ankylosing spondylitis, Anti-GBM/Anti-TBM nephritis, Antiphospholipid syndrome, Autoimmune hepatitis, Autoimmune inner ear disease, Atopic dermatitis, Asthma, Castleman disease, Celiac disease, Chagas disease, Chronic recurrent multifocal osteomyelitis, Cogan's syndrome, Cold agglutinin disease, CREST syndrome, Crohn's disease, Dermatomyositis, Devic's disease (neuromyelitis optica), Discoid lupus, Endometriosis, Eosinophilic esophagitis, Eosinophilic fasciitis, Evan's syndrome, Fibromyalgia, Giant cell arteritis, Giant cell myocarditis, Glomerulonephritis, Goodpasture's syndrome, Granulomatosis with polyangiitis, Graves' disease, Guillain-Barre syndrome, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Juvenile arthritis, Juvenile/Type 1 Diabetes, Juvenile myositis, Kawasaki syndrome (Kawasaki Disease (and/or, e.g., Kawasaki disease shock syndrome (KDSS))), Lichen planus, Lichen sclerosis, Lupus (SLE), Meniere's disease, Multiple sclerosis, Myasthenia gravis, Microscopic polyangiitis, Optic neuritis, Pemphigus, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Sjogren's syndrome, Temporal arteritis/Giant cell arteritis, Transverse myelitis, Ulcerative colitis, Uveitis, Vasculitis, Vitiligo, Viral myocarditis, or Wegener's granulomatosis (Granulomatosis with Polyangiitis (GPA)). In certain embodiments, the IL-1β mediated disease or condition comprises Severe Acute Respiratory Syndrome (SARS), influenza, or a respiratory syncytial viral infection. In certain embodiments, the IL-1β mediated disease or condition comprises a coronavirus (e.g., SARS-CoV-2). In certain embodiments, the IL-1β mediated disease or condition comprises SARS-CoV-2 infection. In certain embodiments, the IL-1β mediated disease or condition is COVID-19.

In some embodiments, the subject treated according to the methods provided herein has a TNFα mediated disease or condition. In some embodiments, the TNFα mediated disease or condition is Severe Acute Respiratory Syndrome (SARS), influenza, respiratory syncytial viral infection, rheumatoid arthritis, juvenile chronic arthritis, Crohn's disease (CD), Ulcerative Colitis (UC), ankylosing spondylitis, psoriasis, multiple sclerosis, atherosclerosis, myocardial infarction, heart failure, myocarditis, cardiac allograft rejection, asthma, ischemic renal injury, renal transplant rejection, glomerulonephritis, or inflammatory eye disease. In some embodiments, the TNFα mediated disease or condition is Severe Acute Respiratory Syndrome (SARS), influenza, or a respiratory syncytial viral infection. In certain embodiments, the TNFα mediated disease or condition comprises a coronavirus infection (e.g., MERS, SARS (such as SARS-CoV-2)). In certain embodiments, the TNFα mediated disease or condition comprises SARS-CoV-2 infection. In certain embodiments, the TNFα mediated disease or condition is COVID-19.

In some embodiments, the subject treated according to the methods provided herein has secondary hemophagocytic lymphohistiocytosis (sHLH).

In some embodiments, the subject treated according to the methods provided herein has a COVID-Related Complication (CRC). In some embodiments, the CRC comprises acute respiratory distress syndrome (ARDS), arrhythmia, shock, acute kidney injury, acute cardiac injury, liver dysfunction and/or secondary infection. In some embodiments, the subject treated according to the methods provided herein has ARDS.

In some embodiments, the methods provided herein further comprise administering to the subject an additional therapy. In some embodiments, the additional therapy comprises the standard of care for the disease being treated (e.g., a coronavirus infection, such as a MERS or SARS (e.g., SARS-CoV-2) infection). In some embodiments, the methods provided herein further comprise administering to the subject an antiviral medication. In some embodiments, the methods provided herein further comprise administering to the subject an antiviral medication such as ribavirin, neuraminidase inhibitor, protease inhibitor, recombinant interferons, antibodies, oseltamivir, zanamivir, peramivir or baloxavir marboxil. In some embodiments, the method further comprises administering to the subject hydroxychloroquine and/or chloroquine. In some embodiments, the method further comprises administering to the subject remdesivir. In some embodiments, the method further comprises administering to the subject an angiotensin-converting enzyme (ACE) inhibitor. In some embodiments, the method further comprises administering to the subject an angiotensin-converting enzyme 2 (ACE2) inhibitor. In some embodiments, the method further comprises administering to the subject plasma from a subject who has recovered from infection by the same virus that is infecting the subject (e.g., plasma from a subject who has recovered from SARS-CoV-2 infection) (e.g., convalescent plasma therapy). In some embodiments, the method further comprises administering (e.g., orally administering) to the subject an anti-inflammatory agent such as an NSAID or an anti-inflammatory steroid. In some embodiments, the method further comprises administering (e.g., orally or intravenously administering) to the subject a corticosteroid such as dexamethasone, prednisone, methylprednisolone, or hydrocortisone. In some embodiments, the method further comprises administering (e.g., orally or intravenously administering) to the subject dexamethasone. In some embodiments, the method further comprises administering to the subject IFN-β1a (e.g., by inhalation). In some embodiments, the method further comprises administering to the subject SNG001 (IFN-β1a for nebulisation).

In some embodiments, the method further comprises administering to the subject an antibody specific for IL-6 and/or the IL-6 receptor. In some embodiments, the method comprises administering to the subject tocilizumab (Actemra®). In some embodiments, the method comprises administering to the subject sarilumab (Kevzara®).

In some embodiments, the method further comprises administering to the subject a monoclonal antibody treatment. In some embodiments, the method further comprises administering to the subject a monoclonal antibody treatment such as bamlanivimab, casirivimab, or imdevimab, or a combination thereof, e.g., a combination of casirivimab and imdevimab. In some embodiments, the additional therapy can comprise a monoclonal antibody treatment such as bamlanivimab, casirivimab, or imdevimab, or a combination thereof, e.g., a combination of casirivimab and imdevimab. In some embodiments, the method further comprises administering to the subject a monoclonal antibody treatment such as bamlanivimab or etesevimab, or a combination of bamlanivimab or etesevimab.

In some embodiments, the additional therapy can comprise budesonide, e.g., inhaled budesonide.

In some embodiments, the method further comprises administering to the subject baricitinib.

In some embodiments, the method further comprises administering to the subject baricitinib in combination with remdesivir.

In some embodiments, the method further comprises administering to the subject an anticoagulation drug, such as heparin or enoxaparin (e.g., a low-dose thereof).

In some embodiments, the method further comprises administering to the subject vitamin D.

In some embodiments, the method further comprises administering to the subject plitidepsin (also referred to as dehydrodidemnin B) (e.g., marketed as Aplidin).

In some embodiments, the method further comprises administering to the subject ivermectin.

In certain aspects, provided herein is a method of identifying a subject at risk for increased severity of a disease or condition (e.g., increased symptom severity associated with a viral infection and/or increased symptom severity associated with an IL8, IL-6, IL-1β and/or TNFα mediated disease or condition) comprising determining expression levels IL-8, IL-6, IL-1β, and/or TNFα in a sample from the subject (e.g., a blood sample contacted with LPS), wherein elevated expression of IL-8, IL-6, IL-1β, and/or TNFα indicates that the subject is at risk for increased severity of the disease or condition. Expression can be elevated as compared to a standard, such as the mean or median level of expression of the cytokine in a cohort of healthy subjects or a cohort of subjects who have not been diagnosed with a viral infection or historical levels. In some embodiments, the method further comprises treating the subject for the disease or condition (e.g., using a method provided herein). In some embodiments, the disease or condition comprises cytokine storm syndrome (cytokine release syndrome) (e.g., a cytokine storm resulting from a viral infection, such as a SARS-CoV-2 infection). In some embodiments, the disease or condition comprises Severe Acute Respiratory Syndrome (SARS), influenza, or a respiratory syncytial viral infection. In certain embodiments, the disease or condition comprises a coronavirus infection (e.g., MERS, SARS (such as SARS-CoV-2)). In certain embodiments, the disease or condition comprises SARS-CoV-2 infection. In some embodiments, the disease or condition is COVID-19.

### BRIEF DESCRIPTION OF THE FIGURES

FIG. 1 is a graph showing the effects of orally-administered Prevotella smEVs (EVs) and intraperitoneally-administered anti-TNFα antibody in a delayed type hypersensitivity (DTH) model of inflammation. Inflammation is assessed as change in ear thickness (mm).

FIGS. 2A and 2B are two panels. FIG. 2A is a graph showing 24 hour DTH measurements after treatment with Prevotella smEVs (Prevotella EV) or dexamethasone. Inflammation is assessed as change in ear thickness (mm). FIG. 2B is a graph showing spleen weight (mg) after treatment with Prevotella smEVs (Prevotella EV) or dexamethasone.

FIGS. 3A-3D are four panels. FIG. 3A is a graph showing 24 hour DTH measurements after treatment with Prevotella smEVs (Prevotella EV). Inflammation is assessed as change in ear thickness (mm). FIGS. 3B-3D are graphs showing levels of IFN-γ (FIG. 3B), IFN-α (FIG. 3C), IFN-β (FIG. 3D) in spleen cells (pg/ml) after treatment with Prevotella smEVs (Prevotella EV).

FIGS. 4A-4D are four panels. FIG. 4A is a graph showing the proportion of TH1 T cells, which are defined by their expression of the transcription factor T-bet (% T-bet positive), of total CD4 T cells in mice treated with either vehicle or Prevotella histicola smEVs (Prevotella EV). FIGS. 4B and 4C are graphs showing amounts (pg/ml) of IFN-γ (FIG. 4B) and TNF (FIG. 4C) from CD4 T cells treated with either vehicle or Prevotella histicola smEVs (Prevotella EV). FIG. 4D is a graph showing levels (pg/ml) of TNF produced by PMA/ionomycin-treated spleen cells from vehicle- or Prevotella histicola smEV (Prevotella EV)-treated mice.

FIGS. 5A-5D are four panels. FIG. 5A is a graph showing IFNγ response (pg/ml) by DCs to CEF after treatment with three doses of Prevotella histicola smEVs (Prevotella EV) compared to the DC+CD8 T cell+CEF peptide co-culture control. FIGS. 5B-5D are graphs showing IL-12p70 (FIG. 5B), TNFα (FIG. 5C), and IL-6 (FIG. 5D) cytokine levels (pg/ml) produced primarily by DCs after treatment with three doses of Prevotella histicola smEVs (Prevotella EV), compared to the DC+CD8 T cell+CEF peptide control.

### DETAILED DESCRIPTION

### General

The disclosure provides Prevotella histicola EVs, and solutions, dried forms and therapeutic compositions that contain extracellular vesicles (EVs) from Prevotella histicola bacteria, and methods for preparing and using the same.

In certain aspects, provided herein is a method of reducing IL-8, IL-6, IL-10, and/or TNFα expression levels in a subject in need thereof, comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, the extracellular vesicles from the Prevotella histicola strain are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain aspects, provided herein is a method of treating a viral infection in a subject comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, the extracellular vesicles from the Prevotella histicola strain are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329). In some embodiments, the viral infection is a coronavirus infection, an influenza infection, and/or a respiratory syncytial virus infection. In some embodiments, the viral infection is a SARS-CoV-2 infection.

In certain aspects, provided herein is a method of treating COVID-19 in a subject comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, the extracellular vesicles from the Prevotella histicola strain are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain aspects, provided herein is a method of treating and/or reducing the severity of cytokine storm syndrome (cytokine release syndrome) (e.g., a cytokine storm resulting from a viral infection, such as a SARS-CoV-2 infection) in a subject comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, the extracellular vesicles from the Prevotella histicola strain are administered in a pharmaceutical composition and/or a solid dosage form. In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is Prevotella histicola Strain B (NRRL accession number B 50329).

In certain embodiments, the therapeutic effects of these orally delivered medicines come from their action on pattern recognition receptors on immune cells in the lining of the small intestine. These cells, in turn, modulate immune cells circulating throughout the body. In some embodiments, the extracellular vesicles are gut-restricted.

The small intestinal axis is a network of anatomical and functional connections linking the small intestine with the rest of the body. It senses external signals in the gut lumen and translates them into systemic immune effects. We have previously shown that an oral microbial drug candidate induces anti-inflammatory activity in preclinical models of inflammation by acting directly on host cells without colonization of the gut or modulation of the microbiome. We now extend these observations to Prevotella histicola EVs, that have potent anti-inflammatory activity in preclinical models. EVs are non-replicating bacterial membrane vesicles with approximately 1/1000th the volume of the parent cell. Prevotella histicola EVs that are orally delivered and gut-restricted in distribution act by modulation of innate and adaptive immunity within the small intestine to attenuate systemic inflammatory responses.

As disclosed herein, this immune connectivity between the small intestine and the rest of the body suggests a possible therapeutic approach for diseases in which the host inflammatory response becomes overwhelming, such as bacterial septic shock and morbidity and mortality associated with viral infections, including flu (influenza) and SARS-CoV-2.

As disclosed herein, extracellular vesicles from Prevotella histicola Strain B (NRRL accession number B 50329) can be useful for the down-regulation of host responses to viral infection.

The disclosure also provides Prevotella histicola EVs, and solutions, dried forms and therapeutic compositions that contain extracellular vesicles (EVs) from Prevotella histicola bacteria, and methods for preparing and using the same.

The disclosure provides solutions and dried forms that contain extracellular vesicles (EVs) from Prevotella histicola bacteria, and methods for preparing and using the same. The disclosure also provides therapeutic compositions that contain the solutions and/or dried forms. In some embodiments, EVs are secreted (for example, produced) by bacterial cells in culture. Such secreted extracellular vesicles may be referred to as secreted microbial extracellular vesicles (smEVs). In some embodiments, EVs are prepared (for example, artificially prepared) by processing bacterial cells, for example, by methods that disrupt the bacterial membrane, such as sonication. Such artificially prepared may be referred to as processed microbial extracellular vesicles (pmEVs).

As used herein, a “dried form” that contains extracellular vesicles (EVs) (for example, from Prevotella histicola bacteria) refers to the product resulting from drying a solution that contains EVs. In some embodiments, the drying is performed, for example, by freeze drying (lyophilization) or spray drying. In some embodiments, the dried form is a powder. As used herein, a powder refers to a type of dried form and includes a lyophilized powder and a spray-dried powder, obtained by a method such as spray drying.

When freeze drying (lyophilization) is performed, the resulting dried form is a lyophilate. In some embodiments, the dried form is a lyophilate. For example, in some embodiments, a lyophilate is a lyophilized powder or a lyophilized cake. In some embodiments, the lyophilized cake is milled to produce a lyophilized powder.

In some embodiments, the solutions and dried forms that contain EVs from Prevotella histicola bacteria also comprise one or more excipients, such as a bulking agent, and/or a lyoprotectant.

In some embodiments, bulking agents and lyoprotectants are used when preparing extracellular vesicles (EVs) for freeze drying. In some embodiments, bulking agents, including but not limited to sucrose, mannitol, polyethylene glycol (PEG, such as PEG 6000), cyclodextrin, maltodextrin, and dextran (such as dextran 40k), are added (for example, as a stock containing the same) to a liquid preparation of EVs (for example, obtained by isolating EVs from a bacterial culture) to prepare a dried form such as a lyophilate, making it easier to handle (and optionally, further formulate, for example, into a therapeutic composition) after drying. In some embodiments, lyoprotectants, including but not limited to trehalose, sucrose, and lactose, are added (for example, as a stock containing the same) to a liquid preparation of EVs (for example, obtained by isolating EVs from a bacterial culture) to protect the EVs while lyophilizing or spray drying. In some embodiments, a bulking agent and/or lyoprotectant is included from an excipient stock that is added to EVs (for example, purified and/or concentrated EVs) to produce a solution, and/or to produce a dried form upon subsequent drying, for example, of the solution. In some embodiments, a dried form such as a lyophilate contains between about 5% and about 100% EV solids by weight. In some embodiments, prior to drying (such as by lyophilization), the total solids, including EVs and excipients, are between about 2% and about 20% by weight.

As described herein, in some embodiments, in a lyophilate containing Prevotella histicola EVs, the excipients make up about 95% to about 99% of the total mass of the powder or cake.

As described herein, in some embodiments, in a lyophilate containing Prevotella histicola EVs, the EVs make up about 2% to about 6% (for example, about 2% to about 5%, about 2% to about 3%, or about 3% to about 5%) of the total mass of the lyophilate.

In some embodiments, the excipient functions to maintain EV efficacy and/or decrease drying (for example, lyophilization) cycle time. In some embodiments, lyoprotectants protect EVs (for example, protein components thereof) during the freeze-drying process. In some embodiments, bulking agents improve the lyophilate properties, for example, for further downstream processing (such as milling, blending, and/or preparing therapeutic compositions).

The length of the lyophilization cycle is important for cost considerations. Critical temperature modifiers such as bulking agents and/or lyoprotectants can significantly reduce drying time. In some embodiments, an excipient stock containing one or more excipients (for example, that contain a bulking agent and/or lyoprotectant) is added to concentrated EVs (for example, a liquid preparation thereof) to bring the total solids to between about 2% to about 20%. In some embodiments, the EVs are concentrated to 5 to 100 times or volume concentration factors (VCF). Examples provided herein targeted about 10% total solids with actual dissolved solids ranging from about 6% to about 8%. In some embodiments, an excipient stock containing one or more excipients (for example, that contain a bulking agent and/or lyoprotectant) (for example, a stock comprising excipients of a formula provided in one of Tables A, B, C, D, K, or P) is prepared as a stock solution in deionized water and sterile filtered with a 0.2 mm filter prior to use. In some embodiments, the stock solution is added to the concentrated EVs, for example, based on weight up to 80%. In some embodiments, the percentage to add is based on the estimated solids contribution of EVs plus the dissolved solids of the excipient stock to achieve the desired total solids content prior to lyophilization.

After freeze drying Prevotella histicola EVs (for example, with an excipient that comprises a bulking agent, for example, as described herein), in some embodiments, the resulting lyophilate (for example, lyophilized cake) has a uniform appearance, and is a white to off-white. In some embodiments, the resulting lyophilate (for example, lyophilized cake) obtained after freeze-drying is a white to off-white, fine and smooth granular powder (for example, after milling (for example, grinding) the lyophilized cake). In some embodiments, dynamic light scattering (DLS) is used to obtain the hydrodynamic diameter (Z average, Zₐᵥₑ) of particles present after the lyophilate (for example, lyophilized powder) is resuspended in deionized water or in a buffer such as PBS (for example, 0.1× PBS). In some embodiments, the Zₐᵥₑ is used to quantify the effectiveness of the stabilizer. For example, if the idealized Zₐᵥₑ particle size is 200 nm; therefore, the resuspended EVs with the lowest Zₐᵥₑ closest to this particle size is considered to be sufficiently stabilized. In some embodiments, the particle size ranges, for example, from 130 nm to 300 nm. In some embodiments, dynamic light scattering (DLS) is used to obtain the mean size of the most dominant DLS integrated peak of particles present after the lyophilate (for example, lyophilized powder) is resuspended in deionized water or in a buffer such as PBS (for example, 0.1× PBS). Notably, the mean size of the particles, whether measured by Z average or by the mean size of the most dominant DLS integrated peak, is not necessarily identical to the mean size of the EVs prior to lyophilization. For example, in some embodiments, the mean size of the particles after lyophilization (for example, after the lyophilate is resuspended in deionized water or in a buffer such as PBS (for example, 0.1× PBS)) is larger or smaller than the mean EV size prior to lyophilization, or the mean size after EV isolation or preparation from a bacterial culture (for example, the mean size after gradient purification of EVs from a bacterial culture). Particles in a lyophilate (after a solution containing EVs is lyophilized) contain Prevotella histicola EVs, and may also include other components from the culture media, such as cell debris, LPS, and/or proteins.

A lyophilate obtained after freeze-drying with the excipients and/or conditions provided herein does not have a porous sponge shape. In some embodiments, after milling, the lyophilate obtained after freeze-drying with the excipients and/or conditions provided herein is a white to off-white, fine and smooth granular lyophilate powder.

Also as described herein, use of the excipients provided herein allows a solution comprising Prevotella histicola EVs to be freeze dried at higher temperatures and shorter drying times. For example, the excipients and methods provided herein allow for EVs to be freeze dried in less than 4000 minutes, for example, freeze dried in about 2800 to about 3200 minutes. As another example, in some embodiments, the freezing step is performed in less than 225 minutes, as opposed to 10 to 15 hours (600 to 900 minutes). As another example, in some embodiments, using the excipients and methods provided herein, primary drying is performed at a temperature between about −35° C. to about −20° C., for example, about −20° C., about −25° C., about −30° C. or about −35° C., as opposed to, for example, −50° C. As another example, in some embodiments, using the excipients and methods provided herein, primary drying is performed for about 42 hours or less (for example, 2500 minutes or less), as opposed to, for example, 50-60 hours (3000 to 3600 minutes). In some embodiments, using the excipients and methods provided herein, total dry times are, for example, about 72 hours or less, for example, about 48 to about 72 hours, for example, less than about 48 hours. In some embodiments, using the excipients and methods provided herein, primary drying is performed for about 65 hours or less (for example, about 60 hours or less). In some embodiments, using the excipients and methods provided herein, secondary drying is performed for about 12 hours or less (for example, about 10 to about 12 hours, about 5 to about 10 hours, about 10 hours or less, or about 5 hours or less). As another example, in some embodiments, using the excipients and methods provided herein, secondary drying is performed at a temperature between about +20° C. to about +30° C., for example, room temperature, for example, about +25° C., as opposed to, for example, −20° C. In some embodiments, use of shorter drying times and/or higher drying temperatures makes the lyophilization process for EVs more commercially feasible.

In some embodiments, the lyophilates containing Prevotella histicola EVs described herein (for example, prepared using the excipients and/or methods described herein) are prepared to have a moisture content (for example, as determined by the Karl Fischer method) of below about 10% (for example, below about 9%, below about 8%, below about 7%, below about 6%, below about 5% or below about 4%, for example, about 1% to about 4%, about 1.5% to about 4%, about 2% to about 4%, about 2% to about 3%) upon completion of freeze drying. In some embodiments, by preparing lyophilates to have a moisture content below about 6%, the lyophilate are better suited for downstream processing, for example, for use in a therapeutic composition. In some embodiments, by preparing lyophilates to have a moisture content below about 6%, the lyophilate has improved stability, e.g., upon storage.

As described in the examples provided herein, the moisture content (determined by Karl Fischer) of lyophilates containing Prevotella histicola EVs had moisture contents of between about 1.8% to about 3.8%. Components of the excipient can be selected to obtain the desired moisture content. The drying conditions can be selected to obtain the desired moisture content.

In some embodiments, the lyophilates containing Prevotella histicola EVs described herein (for example, prepared using the excipients and/or methods described herein) are prepared to have a particle numeration of about 3.25 e10 to about 7.77 e10 particles/mg lyophilate. In some embodiments, particle numeration is determined, for example, by NTA, on lyophilate resuspended in water and with use of a Zetaview camera. In some embodiments, the lyophilates containing Prevotella histicola EVs described herein (for example, prepared using the excipients and/or methods described herein) are prepared to have a particle numeration of about 3.25 e10 to about 6.45 e10 particles/mg lyophilate. In some embodiments, particle numeration is determined, for example, by NTA, on lyophilate resuspended in water and with use of a Zetaview camera. Components of the excipient can be selected to obtain the desired particle numeration. The drying conditions can be selected to obtain the desired particle numeration.

In some embodiments, the particles in the lyophilates (for example, lyophilized powders) described herein (for example, prepared using the excipients and/or methods described herein) are prepared to have a hydrodynamic diameter (Z average, Zₐᵥₑ) of about 137.4 nm to about 226.1 nm. In some embodiments, the particles in the lyophilates (for example, lyophilized powders) described herein (for example, prepared using the excipients and/or methods described herein) are prepared to have a hydrodynamic diameter (Z average, Zₐᵥₑ) of about 137.4 nm to about 212.8 nm. In some embodiments, dynamic light scattering (DLS) is used to obtain the hydrodynamic diameter (Z average, Zₐᵥₑ) of particles present after the lyophilate is resuspended in deionized water or in a buffer such as PBS (for example, 0.1× PBS). Components of the excipient can be selected to obtain the desired Zₐᵥₑ. The drying conditions can be selected to obtain the desired Zₐᵥₑ.

In some embodiments, the spray-dried powders containing EVs described herein (for example, prepared using the excipients and/or methods described herein) are prepared to have a moisture content (for example, as determined by the Karl Fischer method) of below about 10% (for example, below about 9%, below about 8%, below about 7%, below about 6%, below about 5% or below about 4%, for example, about 1% to about 4%, about 1.5% to about 4%, about 2% to about 3%) upon completion of spray drying. In some embodiments, by preparing spray-dried powders to have a moisture content below about 6%, the spray-dried powders are better suited for downstream processing, for example, for use in a therapeutic composition. In some embodiments, by preparing spray-dried powders to have a moisture content below about 6%, the spray-dried powder has improved stability, e.g., upon storage.

As described in the examples provided herein, the moisture content (determined by Karl Fischer) of spray-dried powders containing Prevotella histicola EVs had moisture contents of between about 2.54% to about 8.38%. Components of the excipient can be selected to obtain the desired moisture content. The drying conditions can be selected to obtain the desired moisture content.

In some embodiments, the spray-dried powders containing EVs described herein (for example, prepared using the excipients and/or methods described herein) are prepared to have a particle numeration of about 6.7 e8 to about 2.55 e10 particles/mg spray-dried powder. In some embodiments, particle numeration is determined, for example, by NTA using a Zetaview camera.

As described in the examples provided herein, spray-dried powders containing Prevotella histicola EVs had particle numerations of about 8.05 e9 to about 2.e10 particles/mg spray-dried powder. Components of the excipient can be selected to obtain the desired particle numeration. The drying conditions can be selected to obtain the desired particle numeration.

### Definitions

Unless specifically stated or obvious from context, as used herein, the term “or” is understood to be inclusive. Unless specifically stated or obvious from context, as used herein, the terms “a.” “an,” and “the” are understood to be singular or plural.

The term “about” when used before a numerical value indicates that the value may vary within a reasonable range, such as within +10%, ±5% or 1% of the stated value.

“Adjuvant” or “Adjuvant therapy” broadly refers to an agent that affects an immunological or physiological response in a patient or subject (e.g., human). For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (e.g., a therapeutic composition) to a subject. Examples of routes of administration include oral administration, rectal administration, topical administration, inhalation (nasal) or injection. Administration by injection includes intravenous (IV), intramuscular (IM), and subcutaneous (SC) administration. A therapeutic composition described herein can be administered in any form by any effective route, including but not limited to oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In certain embodiments, a therapeutic composition described herein is administered orally, rectally, topically, intravesically, by injection into or adjacent to a draining lymph node, intravenously, by inhalation or aerosol, or subcutaneously. In another embodiment, a therapeutic composition described herein is administered orally or intravenously. In another embodiment, a therapeutic composition described herein is administered intranasally. In another embodiment, a therapeutic composition described herein is administered orally.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refer to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

A “combination” can refer to EVs from one source strain with another agent, e.g., another EV (e.g., from another strain), with bacteria (e.g., of the same or different strain that the EV was obtained from), or with another therapeutic agent. The combination can be in physical co-existence, either in the same material or product or in physically connected products, as well as the temporal co-administration or co-localization of the EVs and other agent.

As used herein, the term “consists essentially of”(or “consisting essentially of”) means limited to the recited elements and/or steps and those that do not materially affect the basic and novel characteristics of the claimed invention.

“Dysbiosis” refers to a state of the microbiota or microbiome of the gut or other body area, including, e.g., mucosal or skin surfaces (or any other microbiome niche) in which the normal diversity and/or function of the host gut microbiome ecological networks (“microbiome”) are disrupted. A state of dysbiosis may result in a diseased state, or it may be unhealthy under only certain conditions or only if present for a prolonged period. Dysbiosis may be due to a variety of factors, including, environmental factors, infectious agents, host genotype, host diet and/or stress. A dysbiosis may result in: a change (e.g., increase or decrease) in the prevalence of one or more bacteria types (e.g., anaerobic), species and/or strains, change (e.g., increase or decrease) in diversity of the host microbiome population composition; a change (e.g., increase or reduction) of one or more populations of symbiont organisms resulting in a reduction or loss of one or more beneficial effects; overgrowth of one or more populations of pathogens (e.g., pathogenic bacteria); and/or the presence of, and/or overgrowth of, symbiotic organisms that cause disease only when certain conditions are present.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size (e.g., in an animal tumor model)).

The term “effective dose” is the amount of the therapeutic composition that is effective to achieve the desired therapeutic response for a particular subject, composition, and mode of administration, with the least toxicity to the subject.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

“Extracellular vesicles” (EVs) may be naturally-produced vesicles derived from bacteria, such as smEVs. EVs are comprised of bacterial lipids and/or bacterial proteins and/or bacterial nucleic acids and/or bacterial carbohydrate moieties, and are isolated from culture supernatant. The natural production of these vesicles can be artificially enhanced (for example, increased) or decreased through manipulation of the environment in which the bacterial cells are being cultured (for example, by media or temperature alterations). Further, EV compositions may be modified to reduce, increase, add, or remove bacterial components or foreign substances to alter efficacy, immune stimulation, stability, immune stimulatory capacity, stability, organ targeting (for example, lymph node), absorption (for example, gastrointestinal), and/or yield (for example, thereby altering the efficacy). As used herein, the term “purified EV composition” or “EV composition” refers to a preparation of EVs that have been separated from at least one associated substance found in a source material (for example, separated from at least one other bacterial component) or any material associated with the EVs in any process used to produce the preparation. It can also refer to a composition that has been significantly enriched for specific components. Extracellular vesicles may also be obtained from mammalian cells and from can be obtained from microbes such as archaea, fungi, microscopic algae, protozoans, and parasites. Extracellular vesicles from any of these sources can be prepared into a solution and/or dried form as described herein. Extracellular vesicles may be artificially-produced vesicles prepared from bacteria, such as pmEVs, for example, obtained by chemically disrupting (for example, by lysozyme and/or lysostaphin) and/or physically disrupting (for example, by mechanical force) bacterial cells and separating the bacterial membrane components from the intracellular components through centrifugation and/or ultracentrifugation, or other methods, can also be prepared into a solution and/or dried form as described herein.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., psoriasis, atopic dermatitis, lupus, scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject's immune system to treat disease (e.g., immune disease, inflammatory disease, metabolic disease, cancer) and includes, for example, checkpoint inhibitors, cancer vaccines, cytokines, cell therapy, CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include number of immune cells (e.g., of a particular immune cell type), bacterial cells, stromal cells, myeloid derived suppressor cells, fibroblasts, metabolites, and level of cytokines.

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR10 and TLR11. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The “internal transcribed spacer” or “ITS” is a piece of non-functional RNA located between structural ribosomal RNAs (rRNA) on a common precursor transcript often used for identification of eukaryotic species in particular fungi. The rRNA of fungi that forms the core of the ribosome is transcribed as a signal gene and consists of the 8S, 5.8S and 28S regions with ITS4 and 5 between the 8S and 5.8S and 5.8S and 28S regions, respectively. These two intercistronic segments between the 18S and 5.8S and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

The term “isolated” or “enriched” encompasses a microbe, an EV (such as a bacterial EV) or other entity or substance that has been (1) separated from at least some of the components with which it was associated when initially produced (whether in nature or in an experimental setting), and/or (2) produced, prepared, purified, and/or manufactured by the hand of man. Isolated bacteria or EVs may be separated from at least about 10%, about 20%, about 30%, about 40%, about 50%, about 60%, about 70%, about 80%, about 90%, or more of the other components with which they were initially associated. In some embodiments, isolated bacteria or EVs are more than about 80%, about 85%, about 90%, about 91%, about 92%, about 93%, about 94%, about 95%, about 96%, about 97%, about 98%, about 99%, or more than about 99% pure, e.g., substantially free of other components.

As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans).

“Metabolite” as used herein refers to any and all molecular compounds, compositions, molecules, ions, co-factors, catalysts or nutrients used as substrates in any cellular or bacterial metabolic reaction or resulting as product compounds, compositions, molecules, ions, co-factors, catalysts or nutrients from any cellular or bacterial metabolic reaction.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state or dysbiotic microbiome. The microbiome may be native to the subject or patient, or components of the microbiome may be modulated, introduced, or depleted due to changes in health state or treatment conditions (e.g., antibiotic treatment, exposure to different microbes). In some aspects, the microbiome occurs at a mucosal surface. In some aspects, the microbiome is a gut microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium such that it increases or decreases virulence.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g., Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a substance is “pure” if it is substantially free of other components. The terms “purify,” “purifying” and “purified” refer to an EV (such as an EV from bacteria) preparation or other material that has been separated from at least some of the components with which it was associated either when initially produced or generated (e.g., whether in nature or in an experimental setting), or during any time after its initial production. An EV preparation or compositions may be considered purified if it is isolated at or after production, such as from one or more other bacterial components, and a purified microbe or bacterial population may contain other materials up to about 10%, about 20%, about 30%, about 40%, about 50%, about 60%, about 70%, about 80%, about 90%, or above about 90% and still be considered “purified.” In some embodiments, purified EVs are more than about 80%, about 85%, about 90%, about 91%, about 92%, about 93%, about 94%, about 95%, about 96%, about 97%, about 98%, about 99%, or more than about 99% pure. EV compositions (or preparations) are, e.g., purified from residual habitat products.

As used herein, the term “purified EV composition” or “EV composition” refers to a preparation that includes EVs from bacteria that have been separated from at least one associated substance found in a source material (e.g., separated from at least one other bacterial component) or any material associated with the EVs in any process used to produce the preparation. It also refers to a composition that has been significantly enriched or concentrated. In some embodiments, the EVs are concentrated by 2 fold, 3-fold, 4-fold, 5-fold, 10-fold, 100-fold, 1000-fold, 10,000-fold or more than 10,000 fold.

“Residual habitat products” refers to material derived from the habitat for microbiota within or on a subject. For example, fermentation cultures of microbes can contain contaminants, e.g., other microbe strains or forms (e.g., bacteria, virus, mycoplasm, and/or fungus). For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the culture or human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a culture contaminant or a human or animal and that only microbial cells are detectable. In one embodiment, substantially free of residual habitat products may also mean that the microbial composition contains no detectable viral (including bacteria, viruses (e.g., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g., PCR, DNA sequencing), serology and antigen analysis, enzymatic and metabolic analysis, and methods using instrumentation such as flow cytometry with reagents that distinguish desired constituents from contaminants.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any animal. A subject or a patient described as “in need thereof” refers to one in need of a treatment for a disease. Mammals (i.e., mammalian animals) include humans, laboratory animals (e.g., primates, rats, mice), livestock (e.g., cows, sheep, goats, pigs), and household pets (e.g., dogs, cats, rodents). For example, the subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject or patient may be healthy, or may be suffering from (or at increased risk of developing) an immune disorder at any developmental stage or from (or at an increased risk of developing) an infection. In certain embodiments, the subject is a human. For example, a “subject in need thereof” can be, e.g., a subject who has been diagnosed with a viral infection and/or experiencing a symptom of a viral infection, e.g., a viral infection described herein, a bacterial infection, and/or a subject experiencing a symptom of a cytokine release syndrome, and/or a subject having an exaggerated host cytokine response, e.g., as determined by change from baseline in a cytokine level (such as IL-8, IL-6, IL-1β, and/or TNFα), e.g., at day 4 and/or day 7.

As used herein, the term “therapeutic agent” refers to an agent for therapeutic use. In some embodiments, a therapeutic agent is a composition comprising EVs (“an EV composition”) that can be used to treat and/or prevent a disease and/or condition. In some embodiments, the therapeutic agent is a pharmaceutical agent. In some embodiments, a medicinal product, medical food, a food product, or a dietary supplement comprises a therapeutic agent. In some embodiments, the therapeutic agent is in a solution, and in other embodiments, a dried form. The dried form embodiments may be produced, for example, by lyophilization or spray drying. In some embodiments, the dried form of the therapeutic agent is a lyophilized cake or powder. In some embodiments, the dried form of the therapeutic agent is a spray-dried powder.

As used herein, the term “therapeutic composition” or “pharmaceutical composition” refers to a composition that comprises a therapeutically effective amount of a therapeutic agent (for example an EV composition described herein). In some embodiments, the therapeutic composition is (or is present in) a medicinal product, medical food, a food product, or a dietary supplement.

As used herein, the term “treating” a disease in a subject or “treating” a subject having or suspected of having a disease refers to subjecting the subject to a pharmaceutical treatment, e.g., the administration of one or more agents, such that at least one symptom of the disease is decreased or prevented from worsening. For example, “treating” may decrease the level of IL-8, IL-6, IL-1β, and/or TNFα in a subject, e.g., as compared to the level prior to treatment; “treating” may prevent an increase (or cause a decrease) in the level of IL-8, IL-6, IL-1β, and/or TNFα in a subject as compared to a standard, e.g., as compared to the level prior to treatment; “treating” may decrease a clinical factor, such as time on a ventilator or duration of hospitalization as compared to a standard, e.g., as compared to the time or duration in a cohort of subjects who did not receive the treatment. Thus, in one embodiment, “treating” refers inter alia to delaying progression, expediting remission, inducing remission, augmenting remission, speeding recovery, increasing efficacy of or decreasing resistance to alternative therapeutics, or a combination thereof. As used herein, the term “preventing” a disease in a subject refers to administering to the subject to a pharmaceutical treatment, e.g., the administration of one or more agents, such that onset of at least one symptom of the disease is delayed or prevented.

### Bacteria

In certain aspects, provided herein are Prevotella histicola extracellular vesicles (EVs), and solutions and/or dried forms, and therapeutic compositions, that comprise Prevotella histicola extracellular vesicles (EVs). In certain aspects, provided herein are solutions and/or dried forms, and therapeutic compositions, that comprise EVs obtained from Prevotella histicola bacteria.

In certain aspects, provided herein are methods of treating a viral infection (or a bacterial septic shock) and pharmaceutical compositions (e.g., a solid dosing form) comprising extracellular vesicles from Prevotella histicola strain provided herein and methods of treating an IL-8, IL-6, IL-1β, and/or TNFα-mediated disease or condition using such extracellular vesicles from Prevotella histicola strains. In some embodiments, the Prevotella strain is a strain of Prevotella histicola. In some embodiments, the Prevotella strain is Prevotella histicola Strain B (NRRL accession number B 50329). In some embodiments, the Prevotella strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic, 16S or CRISPR nucleotide sequence) of the Prevotella histicola Strain B (NRRL accession number B 50329). In some embodiments, the Prevotella strain is Prevotella histicola Strain B (NRRL accession number B 50329).

In certain aspects, provided herein are methods of reducing IL-8, IL-6, IL-1β, and/or TNFα levels in a subject and pharmaceutical compositions (e.g., a solid dosage form) comprising extracellular vesicles from a Prevotella histicola strain provided herein. In some embodiments, the Prevotella strain is a strain of Prevotella histicola. In some embodiments, the Prevotella strain is Prevotella histicola Strain B (NRRL accession number B 50329). In some embodiments, the Prevotella strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic, 16S or CRISPR nucleotide sequence) of Prevotella histicola Strain B (NRRL accession number B 50329). In some embodiments, the Prevotella strain is Prevotella histicola Strain B (NRRL accession number B 50329).

In certain aspects, provided herein are Prevotella histicola extracellular vesicles (EVs), and solutions and/or powders, and therapeutic compositions, that comprise Prevotella histicola extracellular vesicles (EVs). In certain aspects, provided herein are solutions and/or powders, and therapeutic compositions, that comprise EVs obtained from Prevotella histicola bacteria.

In some embodiments, Prevotella histicola bacteria from which EVs are obtained are lyophilized.

In some embodiments, Prevotella histicola bacteria from which EVs are obtained are gamma irradiated (e.g., at 17.5 or 25 kGy).

In some embodiments, Prevotella histicola bacteria from which EVs are obtained are UV irradiated.

In some embodiments, Prevotella histicola bacteria from which EVs are obtained are heat inactivated (e.g., at 50° C. for two hours or at 90° C. for two hours).

In some embodiments, Prevotella histicola bacteria from which EVs are obtained are acid treated.

In some embodiments, Prevotella histicola bacteria from which EVs are obtained are oxygen sparged (e.g., at 0.1 vvm for two hours).

In some embodiments, the Prevotella histicola EVs are lyophilized.

In some embodiments, the Prevotella histicola EVs are gamma irradiated (e.g., at 17.5 or 25 kGy).

In some embodiments, the Prevotella histicola EVs are UV irradiated.

In some embodiments, the Prevotella histicola EVs are heat inactivated (e.g., at 50° C. for two hours or at 90° C. for two hours).

In some embodiments, the Prevotella histicola EVs are acid treated.

In some embodiments, the Prevotella histicola EVs are oxygen sparged (e.g., at 0.1 vvm for two hours).

In some embodiments, the Prevotella histicola EVs are lyophilized.

In some embodiments, the Prevotella histicola EVs are spray dried.

In some embodiments, the Prevotella histicola EVs are gamma irradiated (e.g., at 17.5 or 25 kGy).

In some embodiments, the Prevotella histicola EVs are UV irradiated.

In some embodiments, the Prevotella histicola EVs are heat inactivated (e.g., at 50° C. for two hours or at 90° C. for two hours).

In some embodiments, the Prevotella histicola EVs are acid treated.

In some embodiments, the Prevotella histicola EVs are oxygen sparged (e.g., at 0.1 vvm for two hours).

The phase of growth can affect the amount or properties of bacteria and/or EVs produced by Prevotella histicola bacteria. For example, in the methods of EVs preparation provided herein, EVs can be isolated, e.g., from a culture, at the start of the log phase of growth, midway through the log phase, and/or once stationary phase growth has been reached.

In some embodiments, the Prevotella histicola EVs are from one strain of bacteria, e.g., a strain provided herein.

In some embodiments, the Prevotella histicola EVs are from one strain of bacteria (e.g., a strain provided herein) or from more than one strain.

In some embodiments, the EVs are from Prevotella histicola bacteria, e.g., from a strain comprising at least 90% or at least 99% genomic, 16S and/or CRISPR sequence identity to the nucleotide sequence of the Prevotella Strain B 50329 (NRRL accession number B 50329). In some embodiments, the EVs are from Prevotella histicola bacteria, e.g., from Prevotella Strain B 50329 (NRRL accession number B 50329).

In some embodiments, the Prevotella histicola bacteria from which the EVs are obtained are modified (e.g., engineered) to reduce toxicity or other adverse effects, to enhance delivery) (e.g., oral delivery) of the EVs (e.g., by improving acid resistance, muco-adherence and/or penetration and/or resistance to bile acids, digestive enzymes, resistance to anti-microbial peptides and/or antibody neutralization), to target desired cell types (e.g., M-cells, goblet cells, enterocytes, dendritic cells, macrophages), to enhance their immunomodulatory and/or therapeutic effect of the EVs (e.g., either alone or in combination with another therapeutic agent), and/or to enhance immune activation or suppression by the EVs (e.g., through modified production of polysaccharides, pili, fimbriae, adhesins). In some embodiments, the engineered bacteria described herein are modified to improve EV manufacturing (e.g., higher oxygen tolerance, stability, improved freeze-thaw tolerance, shorter generation times). For example, in some embodiments, the engineered bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may results in the overexpression and/or underexpression of one or more genes. The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

### Modified EVs

In some aspects, the Prevotella histicola EVs described herein are modified such that they comprise, are linked to, and/or are bound by a therapeutic moiety.

In some embodiments, the Prevotella histicola EVs described herein are engineered such that they comprise, are linked to, and/or are bound by a magnetic and/or paramagnetic moiety (e.g., a magnetic bead). In some embodiments, the magnetic and/or paramagnetic moiety is comprised by and/or directly linked to the bacteria. In some embodiments, the magnetic and/or paramagnetic moiety is linked to and/or a part of an EV-binding moiety that that binds to the EV. In some embodiments, the EV-binding moiety is a fragment of or a full-length peptidoglycan recognition protein, such as PGRP. In some embodiments the EV-binding moiety has binding specificity for the EV (e.g., by having binding specificity for a bacterial antigen). In some embodiments, the EV-binding moiety comprises an antibody or antigen binding fragment thereof. In some embodiments, the EV-binding moiety comprises a T cell receptor or a chimeric antigen receptor (CAR).

### Production of Bacterial Extracellular Vesicles (EVs)

Secreted EVs. In certain aspects, the EVs (such as secreted EVs (smEVs) from bacteria described herein) are prepared using any method known in the art.

In some embodiments, the smEVs are prepared without an smEV purification step. For example, in some embodiments, bacteria described herein are killed using a method that leaves the smEVs intact and the resulting bacterial components, including the smEVs, are used in the methods and compositions described herein. In some embodiments, the bacteria are killed using an antibiotic (for example, using an antibiotic described herein). In some embodiments, the bacteria are killed using UV irradiation. In some embodiments, the bacteria are heat-killed.

In some embodiments, the smEVs described herein are purified from one or more other bacterial components. Methods for purifying smEVs from bacteria are known in the art. In some embodiments, smEVs are prepared from bacterial cultures using methods described in S. Bin Park, et al. PLoS ONE. 6(3):e17629 (2011) or G. Norheim, et al. PLoS ONE. 10(9): e0134353 (2015) or Jeppesen, et al. Cell 177:428 (2019), each of which is hereby incorporated by reference in its entirety. In some embodiments, the bacteria are cultured to high optical density and then centrifuged to pellet bacteria (for example, at 10,000×g for 30 min at 4° C., at 15,500×g for 15 min at 4° C.). In some embodiments, the culture supernatants are then passed through filters to exclude intact bacterial cells (for example, a 0.22 μm filter). In some embodiments, the supernatants are then subjected to tangential flow filtration, during which the supernatant is concentrated, species smaller than 100 kDa are removed, and the media is partially exchanged with PBS. In some embodiments, filtered supernatants are centrifuged to pellet bacterial smEVs (for example, at 100,000-150,000×g for 1-3 hours at 4° C., at 200,000×g for 1-3 hours at 4° C.). In some embodiments, the smEVs are further purified by resuspending the resulting smEV pellets (for example, in PBS), and applying the resuspended smEVs to an Optiprep (iodixanol) gradient or gradient (for example, a 30-60% discontinuous gradient, a 0-45% discontinuous gradient), followed by centrifugation (for example, at 200,000×g for 4-20 hours at 4° C.). smEV bands can be collected, diluted with PBS, and centrifuged to pellet the smEVs (for example, at 150,000×g for 3 hours at 4° C., at 200,000×g for 1 hour at 4° C.). The purified smEVs can be stored, for example, at −80° C. or −20° C. until use. In some embodiments, the smEVs are further purified by treatment with DNase and/or proteinase K.

For example, in some embodiments, cultures of bacteria can be centrifuged at 11,000×g for 20-40 min at 4° C. to pellet bacteria. Culture supernatants may be passed through a 0.22 μm filter to exclude intact bacterial cells. Filtered supernatants may then be concentrated using methods that may include, but are not limited to, ammonium sulfate precipitation, ultracentrifugation, or filtration. For example, for ammonium sulfate precipitation, 1.5-3 M ammonium sulfate can be added to filtered supernatant slowly, while stirring at 4° C. Precipitations can be incubated at 4° C. for 8-48 hours and then centrifuged at 11,000×g for 20-40 min at 4° C. The resulting pellets contain bacteria smEVs and other debris. Using ultracentrifugation, filtered supernatants can be centrifuged at 100,000-200,000×g for 1-16 hours at 4° C. The pellet of this centrifugation contains bacterial smEVs and other debris such as large protein complexes. In some embodiments, using a filtration technique, such as through the use of an Amicon Ultra spin filter or by tangential flow filtration, supernatants can be filtered so as to retain species of molecular weight>50 or 100 kDa.

Alternatively, smEVs can be obtained from bacteria cultures continuously during growth, or at selected time points during growth, for example, by connecting a bioreactor to an alternating tangential flow (ATF) system (for example, XCell ATF from Repligen). The ATF system retains intact cells (>0.22 μm) in the bioreactor, and allows smaller components (for example, smEVs, free proteins) to pass through a filter for collection. For example, the system may be configured so that the <0.22 μm filtrate is then passed through a second filter of 100 kDa, allowing species such as smEVs between 0.22 μm and 100 kDa to be collected, and species smaller than 100 kDa to be pumped back into the bioreactor. Alternatively, the system may be configured to allow for medium in the bioreactor to be replenished and/or modified during growth of the culture. smEVs collected by this method may be further purified and/or concentrated by ultracentrifugation or filtration as described above for filtered supernatants.

smEVs obtained by methods provided herein may be further purified by size-based column chromatography, by affinity chromatography, by ion-exchange chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in PBS and 3 volumes of 60% Optiprep are added to the sample. In some embodiments, if filtration was used to concentrate the filtered supernatant, the concentrate is diluted using 60% Optiprep to a final concentration of 35% Optiprep. Samples are applied to a 0-45% discontinuous Optiprep gradient and centrifuged at 200,000×g for 3-24 hours at 4° C., for example, 4-24 hours at 4° C.

In some embodiments, to confirm sterility and isolation of the smEV preparations, smEVs are serially diluted onto agar medium used for routine culture of the bacteria being tested, and incubated using routine conditions. Non-sterile preparations are passed through a 0.22 μm filter to exclude intact cells. To further increase purity, isolated smEVs may be DNase or proteinase K treated.

In some embodiments, for preparation of smEVs used for in vivo injections, purified smEVs are processed as described previously (G. Norheim, et al. PLoS ONE. 10(9): e0134353 (2015)). Briefly, after sucrose gradient centrifugation, bands containing smEVs are resuspended to a final concentration of 50 μg/mL in a solution containing 3% sucrose or other solution suitable for in vivo injection known to one skilled in the art. This solution may also contain adjuvant, for example aluminum hydroxide at a concentration of 0-0.5% (w/v). In some embodiments, for preparation of smEVs used for in vivo injections, smEVs in PBS are sterile-filtered to <0.22 μm.

In certain embodiments, to make samples compatible with further testing (for example, to remove sucrose prior to TEM imaging or in vitro assays), samples are buffer exchanged into PBS or 30 mM Tris, pH 8.0 using filtration (for example, Amicon Ultra columns), dialysis, or ultracentrifugation (200,000×g, ≥3 hours, 4° C.) and resuspension.

In some embodiments, the sterility of the smEV preparations can be confirmed by plating a portion of the smEVs onto agar medium used for standard culture of the bacteria used in the generation of the smEVs and incubating using standard conditions.

In some embodiments, select smEVs are isolated and enriched by chromatography and binding surface moieties on smEVs. In other embodiments, select smEVs are isolated and/or enriched by fluorescent cell sorting by methods using affinity reagents, chemical dyes, recombinant proteins or other methods known to one skilled in the art.

In some embodiments, smEVs are analyzed, for example, as described in Jeppesen, et al. Cell 177:428 (2019).

In some embodiments, smEVs are lyophilized.

In some embodiments, smEVs are spray dried.

In some embodiments, smEVs are gamma irradiated (for example, at 17.5 or 25 kGy).

In some embodiments, smEVs are UV irradiated.

In some embodiments, smEVs are heat inactivated (for example, at 50° C. for two hours or at 90° C. for two hours).

In some embodiments, smEVs are acid treated.

In some embodiments, smEVs are oxygen sparged (for example, at 0.1 vvm for two hours).

The phase of growth can affect the amount or properties of bacteria and/or smEVs produced by bacteria. For example, in the methods of smEV preparation provided herein, smEVs can be isolated, for example, from a culture, at the start of the log phase of growth, midway through the log phase, and/or once stationary phase growth has been reached.

The growth environment (for example, culture conditions) can affect the amount of smEVs produced by bacteria. For example, the yield of smEVs can be increased by an smEV inducer, as provided in Table 4.

TABLE 4
Culture Techniques to Increase smEV Production

| smEV | | |
|---|---|---|
| inducement | smEV inducer | Acts on |
| Temperature | Heat | stress response |
| | RT to 37° C. temp change | simulates infection |
| | 37 to 40° C. temp change | febrile infection |
| ROS | Plumbagin | oxidative stress response |
| | Cumene hydroperoxide | oxidative stress response |
| | Hydrogen Peroxide | oxidative stress response |
| Antibiotics | Ciprofloxacin | bacterial SOS response |
| | Gentamycin | protein synthesis |
| | Polymyxin B | outer membrane |
| | D-cylcloserine | cell wall |
| Osmolyte | NaCl | osmotic stress |
| Metal Ion | Iron Chelation | iron levels |
| Stress | EDTA | removes divalent cations |
| | Low Hemin | iron levels |
| Media | Lactate | growth |
| additives or | Amino acid deprivation | stress |
| removal | Hexadecane | stress |
| | Glucose | growth |
| | Sodium bicarbonate | ToxT induction |
| | PQS | vesiculator (from bacteria) |
| | Diamines + DFMO | membrane anchoring |
| | High nutrients | (negativicutes only) |
| | Low nutrients | enhanced growth |
| Other | Oxygen | oxygen stress in anaerobe |
| mechanisms | No Cysteine | oxygen stress in anaerobe |
| | Inducing biofilm or | |
| | floculation | |
| | Diauxic Growth | |
| | Phage | |
| | Urea | |

In the methods for preparing smEVs provided herein, the methods can optionally include exposing a culture of bacteria to an smEV inducer prior to isolating smEVs from the bacterial culture. The culture of bacteria can be exposed to an smEV inducer at the start of the log phase of growth, midway through the log phase, and/or once stationary phase growth has been reached.

Processed EVs. In certain aspects, the EVs (such as processed EVs (pmEVs) described herein) are prepared (for example, artificially prepared) using any method known in the art.

In some embodiments, the pmEVs are prepared without a pmEV purification step. For example, in some embodiments, bacteria from which the pmEVs described herein are released are killed using a method that leaves the bacterial pmEVs intact, and the resulting bacterial components, including the pmEVs, are used in the methods and compositions described herein. In some embodiments, the bacteria are killed using an antibiotic (for example, using an antibiotic described herein). In some embodiments, the bacteria are killed using UV irradiation.

In some embodiments, the pmEVs described herein are purified from one or more other bacterial components. Methods for purifying pmEVs from bacteria (and optionally, other bacterial components) are known in the art. In some embodiments, pmEVs are prepared from bacterial cultures using methods described in Them et al. (J. Proteome Res. 9(12):6135-6147 (2010)) or Sandrini et al. (Bio-protocol 4(21): e1287 (2014)), each of which is hereby incorporated by reference in its entirety. In some embodiments, the bacteria are cultured to high optical density and then centrifuged to pellet bacteria (for example, at 10,000-15,000×g for 10-15 min at room temperature or 4° C.). In some embodiments, the supernatants are discarded and cell pellets are frozen at −80° C. In some embodiments, cell pellets are thawed on ice and resuspended in 100 mM Tris-HCl, pH 7.5 supplemented with 1 mg/mL DNase I. In some embodiments, cells are lysed using an Emulsiflex C-3 (Avestin, Inc.) under conditions recommended by the manufacturer. In some embodiments, debris and unlysed cells are pelleted by centrifugation at 10,000×g for 15 min at 4° C. In some embodiments, supernatants are then centrifuged at 120,000×g for 1 hour at 4° C. In some embodiments, pellets are resuspended in ice-cold 100 mM sodium carbonate, pH 11, incubated with agitation for 1 hour at 4° C., and then centrifuged at 120,000×g for 1 hour at 4° C. In some embodiments, pellets are resuspended in 100 mM Tris-HCl, pH 7.5, re-centrifuged at 120,000×g for 20 min at 4° C., and then resuspended in 0.1 M Tris-HCl, pH 7.5 or in PBS. In some embodiments, samples are stored at −20° C.

In certain aspects, pmEVs are obtained by methods adapted from Sandrini et al, 2014. In some embodiments, bacterial cultures are centrifuged at 10,000-15,500×g for 10-15 min at room temp or at 4° C. In some embodiments, cell pellets are frozen at −80° C. and supernatants are discarded. In some embodiments, cell pellets are thawed on ice and resuspended in 10 mM Tris-HCl, pH 8.0, 1 mM EDTA supplemented with 0.1 mg/mL lysozyme. In some embodiments, samples are incubated with mixing at room temp or at 37° C. for 30 min. In some embodiments, samples are re-frozen at −80° C. and thawed again on ice. In some embodiments, DNase I is added to a final concentration of 1.6 mg/mL and MgCl₂ to a final concentration of 100 mM. In some embodiments, samples are sonicated using a QSonica Q500 sonicator with 7 cycles of 30 sec on and 30 sec off. In some embodiments, debris and unlysed cells are pelleted by centrifugation at 10,000×g for 15 min. at 4° C. In some embodiments, supernatants are then centrifuged at 110,000×g for 15 min at 4° C. In some embodiments, pellets are resuspended in 10 mM Tris-HCl, pH 8.0, 2% Triton X-100 and incubated 30-60 min with mixing at room temperature. In some embodiments, samples are centrifuged at 110,000×g for 15 min at 4° C. In some embodiments, pellets are resuspended in PBS and stored at −20° C.

In certain aspects, a method of forming (for example, preparing) isolated bacterial pmEVs, described herein, comprises the steps of: (a) centrifuging a bacterial culture, thereby forming a first pellet and a first supernatant, wherein the first pellet comprises cells; (b) discarding the first supernatant;(c) resuspending the first pellet in a solution; (d) lysing the cells; (e) centrifuging the lysed cells, thereby forming a second pellet and a second supernatant; (f) discarding the second pellet and centrifuging the second supernatant, thereby forming a third pellet and a third supernatant; (g) discarding the third supernatant and resuspending the third pellet in a second solution, thereby forming the isolated bacterial pmEVs.

In some embodiments, the method further comprises the steps of: (h) centrifuging the solution of step (g), thereby forming a fourth pellet and a fourth supernatant; (i) discarding the fourth supernatant and resuspending the fourth pellet in a third solution. In some embodiments, the method further comprises the steps of: (j) centrifuging the solution of step (i), thereby forming a fifth pellet and a fifth supernatant; and (k) discarding the fifth supernatant and resuspending the fifth pellet in a fourth solution.

In some embodiments, the centrifugation of step (a) is at 10,000×g. In some embodiments the centrifugation of step (a) is for 10-15 minutes. In some embodiments, the centrifugation of step (a) is at 4° C. or room temperature. In some embodiments, step (b) further comprises freezing the first pellet at −80° C. In some embodiments, the solution in step (c) is 100 mM Tris-HCl, pH 7.5 supplemented with 1 mg/ml DNaseI. In some embodiments, the solution in step (c) is 10 mM Tris-HCl, pH 8.0, 1 mM EDTA, supplemented with 0.1 mg/ml lysozyme. In some embodiments, step (c) further comprises incubating for 30 minutes at 37° C. or room temperature. In some embodiments, step (c) further comprises freezing the first pellet at −80° C. In some embodiments, step (c) further comprises adding DNase I to a final concentration of 1.6 mg/ml. In some embodiments, step (c) further comprises adding MgCl₂ to a final concentration of 100 mM. In some embodiments, the cells are lysed in step (d) via homogenization. In some embodiments, the cells are lysed in step (d) via emulsiflex C3. In some embodiments, the cells are lysed in step (d) via sonication. In some embodiments, the cells are sonicated in 7 cycles, wherein each cycle comprises 30 seconds of sonication and 30 seconds without sonication. In some embodiments, the centrifugation of step (e) is at 10,000×g. In some embodiments, the centrifugation of step (e) is for 15 minutes. In some embodiments, the centrifugation of step (e) is at 4° C. or room temperature.

In some embodiments, the centrifugation of step (f) is at 120,000×g. In some embodiments, the centrifugation of step (f) is at 110,000×g. In some embodiments, the centrifugation of step (f) is for 1 hour. In some embodiments, the centrifugation of step (f) is for 15 minutes. In some embodiments, the centrifugation of step (f) is at 4° C. or room temperature. In some embodiments, the second solution in step (g) is 100 mM sodium carbonate, pH 11. In some embodiments, the second solution in step (g) is 10 mM Tris-HCl pH 8.0, 2% triton X-100. In some embodiments, step (g) further comprises incubating the solution for 1 hour at 4° C. In some embodiments, step (g) further comprises incubating the solution for 30-60 minutes at room temperature. In some embodiments, the centrifugation of step (h) is at 120,000×g. In some embodiments, the centrifugation of step (h) is at 110,000×g. In some embodiments, the centrifugation of step (h) is for 1 hour. In some embodiments, the centrifugation of step (h) is for 15 minutes. In some embodiments, the centrifugation of step (h) is at 4° C. or room temperature. In some embodiments, the third solution in step (i) is 100 mM Tris-HCl, pH 7.5. In some embodiments, the third solution in step (i) is PBS. In some embodiments, the centrifugation of step (j) is at 120,000×g. In some embodiments, the centrifugation of step (j) is for 20 minutes. In some embodiments, the centrifugation of step (j) is at 4° C. or room temperature. In some embodiments, the fourth solution in step (k) is 100 mM Tris-HCl, pH 7.5 or PBS.

pmEVs obtained by methods provided herein may be further purified by size based column chromatography, by affinity chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 35% Optiprep in PBS. In some embodiments, if filtration was used to concentrate the filtered supernatant, the concentrate is diluted using 60% Optiprep to a final concentration of 35% Optiprep. Samples are applied to a 35-60% discontinuous sucrose gradient and centrifuged at 200,000×g for 3-24 hours at 4° C.

In some embodiments, to confirm sterility and isolation of the pmEV preparations, pmEVs are serially diluted onto agar medium used for routine culture of the bacteria being tested, and incubated using routine conditions. Non-sterile preparations are passed through a 0.22 μm filter to exclude intact cells. To further increase purity, isolated pmEVs may be DNase or proteinase K treated.

In some embodiments, the sterility of the pmEV preparations can be confirmed by plating a portion of the pmEVs onto agar medium used for standard culture of the bacteria used in the generation of the pmEVs and incubating using standard conditions.

In some embodiments select pmEVs are isolated and enriched by chromatography and binding surface moieties on pmEVs. In other embodiments, select pmEVs are isolated and/or enriched by fluorescent cell sorting by methods using affinity reagents, chemical dyes, recombinant proteins or other methods known to one skilled in the art.

In some embodiments, pmEVs are analyzed, for example, as described in Jeppesen et al. Cell 177:428 (2019).

In some embodiments, pmEVs are lyophilized.

In some embodiments, pmEVs are spray dried.

In some embodiments, pmEVs are gamma irradiated (for example, at 17.5 or 25 kGy).

In some embodiments, pmEVs are UV irradiated.

In some embodiments, pmEVs are heat inactivated (for example, at 50° C. for two hours or at 90° C. for two hours).

In some embodiments, pmEVs are acid treated.

In some embodiments, pmEVs are oxygen sparged (for example, at 0.1 vvm for two hours).

The phase of growth can affect the amount or properties of bacteria. In the methods of pmEV preparation provided herein, pmEVs can be isolated, for example, from a culture, at the start of the log phase of growth, midway through the log phase, and/or once stationary phase growth has been reached.

### Make-Up of Therapeutic Compositions

In certain embodiments, provided herein are therapeutic compositions (e.g., solid dosage forms) comprising extracellular vesicles from Prevotella histicola bacteria provided herein.

In some embodiments, the therapeutic compositions comprise extracellular vesicles from only one strain of bacteria, e.g., Prevotella histicola, e.g., Prevotella Strain B 50329.

In some embodiments, the Prevotella histicola is Prevotella histicola Strain B (NRRL accession number B 50329). In some embodiments, the Prevotella histicola strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B.

In some embodiments, the therapeutic composition is formulated as a capsule or a tablet. In some embodiments, the therapeutic composition comprises an enteric coating or micro encapsulation. In some embodiments, the therapeutic composition is prepared as a capsule. In some embodiments, the capsule is an enteric coated capsule. In some embodiments, the therapeutic composition is prepared as a tablet. In some embodiments, the tablet is an enteric coated tablet. In some embodiments, the enteric coating allows release of the therapeutic composition in the small intestine, e.g., in the upper small intestine, e.g., in the duodenum. In some embodiments, the therapeutic composition, e.g., pharmaceutical composition is a dried form. The dried form can be resuspended (e.g., in a liquid such as a solution, buffer, water or other beverage or a food), e.g., for administration to a subject.

### Solid Dosage Form Composition

In certain embodiments, provided herein are solid dosage forms (solid dose forms) comprising extracellular vesicles from a Prevotella strain and a pharmaceutically acceptable carrier.

In some embodiments, the therapeutic composition comprising extracellular vesicles from Prevotella histicola bacteria is prepared as a dried form (e.g., for resuspension or for use in a solid dosage form (such as a capsule)) or as a solid dosage form, such as a tablet, a mini-tablet, or a capsule; or a combination of these forms (e.g., mini-tablets comprised in a capsule)). The dried form can comprise a powder (such as a lyophilized powder or spray-dried powder). The powder can comprise lyophilized EVs. The powder can comprise spray-dried EVs. In some embodiments, the powder further comprises mannitol, magnesium stearate, and/or colloidal silicon dioxide. In some embodiments, the extracellular vesicles from Prevotella histicola bacteria are gamma irradiated.

In some embodiments, the solid dosage forms comprise extracellular vesicles from only one strain of bacteria, e.g., Prevotella histicola, e.g., Prevotella Strain B 50329.

The solid dosage form (also referred to as solid dose form herein) can comprise one or more excipients, e.g., pharmaceutically acceptable excipients. The extracellular vesicles from Prevotella histicola bacteria in the solid dosage form can be isolated extracellular vesicles from Prevotella histicola bacteria. Optionally, the extracellular vesicles from Prevotella histicola bacteria in the solid dosage form can be lyophilized. Optionally, the extracellular vesicles from Prevotella histicola bacteria in the solid dosage form are gamma irradiated. The solid dosage form can comprise a tablet. The solid dosage form can comprise a capsule. The solid dosage form can comprise a tablet, a mini-tablet, a capsule, or a powder; or a combination of these forms (e.g., mini-tablets comprised in a capsule).

The extracellular vesicles from Prevotella histicola bacteria in the solid dosage form can be in a dried form (e.g., the dried form comprises lyophilized or spray-dried extracellular vesicles from Prevotella histicola bacteria). The extracellular vesicles from Prevotella histicola bacteria in the solid dosage form can be in a powder (e.g., the powder comprises lyophilized or spray-dried extracellular vesicles from Prevotella histicola bacteria). In some embodiments, the dried form further comprises mannitol, magnesium stearate, and/or colloidal silicon dioxide. In some embodiments, the dried form further comprises mannitol, magnesium stearate, and colloidal silicon dioxide. Optionally, the extracellular vesicles from Prevotella histicola bacteria in the dried form can be lyophilized. Optionally, the extracellular vesicles from Prevotella histicola bacteria in the dried form can be spray dried. Optionally, the Prevotella histicola bacteria in the powder are gamma irradiated.

In some embodiments, the dried form of extracellular vesicles from Prevotella histicola bacteria is resuspended (e.g., in a liquid such as a solution, buffer, water or other beverage or a food), e.g., for administration to a subject.

In certain embodiments, the therapeutic composition (e.g., pharmaceutical composition) provided herein is prepared as a solid dosage form comprising extracellular vesicles from Prevotella histicola bacteria and a pharmaceutically acceptable carrier.

In certain embodiments, the therapeutic composition (e.g., pharmaceutical composition) provided herein is prepared as a solid dosage form comprising extracellular vesicles from Prevotella histicola bacteria and a pharmaceutically acceptable carrier. The solid dosage form can comprise a tablet, a mini-tablet, a capsule, a pill, or a dried form (such as a powder); or a combination of these forms (e.g., mini-tablets comprised in a capsule).

In some embodiments, the solid dosage form described herein can be a capsule, e.g., an enteric coated capsule. In some embodiments, the capsule is enteric coated, e.g., for duodenal release at pH 5.5. The capsule can be, e.g., a size 00, size 0, size 1, size 2, size 3, size 4, or size 5 capsule. In some embodiments, the capsule is a size 0 capsule. In some embodiments, the capsule comprises freeze-dried powder that comprises the extracellular vesicles from Prevotella Strain B. In some embodiments, the capsule comprises spray-dried powder that comprises the extracellular vesicles from Prevotella Strain B.

In some embodiments, the solid dosage form described herein can be, e.g., a tablet or a mini-tablet. In some embodiments, a plurality of mini-tablets can be in (e.g., loaded into) a capsule.

In some embodiments, the solid dosage form comprises a tablet (>4 mm) (e.g., 5 mm-17 mm). In some embodiments, the tablet is enteric coated, e.g., for duodenal release at pH 5.5. For example, the tablet is a 5 mm, 6 mm, 7 mm, 8 mm, 9 mm, 10 mm, 11 mm, 12 mm, 13 mm, 14 mm, 15 mm, 16 mm, 17 mm or 18 mm tablet. In some embodiments, the tablet comprises freeze-dried powder that comprises the extracellular vesicles from Prevotella Strain B. In some embodiments, the tablet comprises spray-dried powder that comprises the extracellular vesicles from Prevotella Strain B.

In some embodiments, the solid dosage form comprises a mini-tablet. In some embodiments, the mini-tablet is enteric coated, e.g., for duodenal release at pH 5.5. The mini-tablet can be in the size range of 1 mm-4 mm range. E.g., the mini-tablet can be a 1 mm mini-tablet, 1.5 mm mini-tablet, 2 mm mini-tablet, 3 mm mini-tablet, or 4 mm mini-tablet. In some embodiments, the mini-tablet comprises freeze-dried powder that comprises the extracellular vesicles from Prevotella Strain B. In some embodiments, the mini-tablet comprises spray-dried powder that comprises the extracellular vesicles from Prevotella Strain B.

As used herein, the size of the tablet, mini-tablet or capsule refers to the size of the tablet, mini-tablet or capsule prior to application of an enteric coating.

In some embodiments, the solid dosage form comprises a mini-tablet. The mini-tablet can be in the size range of 1 mm-4 mm range. E.g., the mini-tablet can be a 1 mm mini-tablet, 1.5 mm mini-tablet, 2 mm mini-tablet, 3 mm mini-tablet, or 4 mm mini-tablet. The size refers to the diameter of the mini-tablet, as is known in the art. As used herein, the size of the mini-tablet refers to the size of the mini-tablet prior to application of an enteric coating.

The mini-tablets can be in a capsule. The capsule can be a size 00, size 0, size 1, size 2, size 3, size 4, or size 5 capsule. The capsule that contains the mini-tablets can comprise a single layer coating, e.g., a non-enteric coating such as gelatin or HPMC. The mini-tablets can be inside a capsule: the number of mini-tablets inside a capsule will depend on the size of the capsule and the size of the mini-tablets. As an example, a size 0 capsule can contain 31-35 (an average of 33) mini-tablets that are 3 mm mini-tablets.

The solid dosage form (e.g., tablet, mini-tablet, or capsule) described herein can be enterically coated. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P).

The solid dose form can comprise a coating. The solid dose form can comprise a single layer coating, e.g., enteric coating, e.g., a Eudragit-based coating, e.g., EUDRAGIT L30 D-55, triethylcitrate, and talc. The solid dose form can comprise two layers of coating. For example, an inner coating can comprise, e.g., EUDRAGIT L30 D-55, triethylcitrate, talc, citric acid anhydrous, and sodium hydroxide, and an outer coating can comprise, e.g., EUDRAGIT L30 D-55, triethylcitrate, and talc. EUDRAGIT is the brand name for a diverse range of polymethacrylate-based copolymers. It includes anionic, cationic, and neutral copolymers based on methacrylic acid and methacrylic/acrylic esters or their derivatives. Eudragits are amorphous polymers having glass transition temperatures between 9 to >150° C. Eudragits are non-biodegradable, nonabsorbable, and nontoxic. Anionic Eudragit L dissolves at pH>6 and is used for enteric coating, while Eudragit S, soluble at pH>7 is used for colon targeting. Eudragit RL and RS, having quaternary ammonium groups, are water insoluble, but swellable/permeable polymers which are suitable for the sustained release film coating applications. Cationic Eudragit E, insoluble at pH≥5, can prevent drug release in saliva.

The solid dose form (e.g., a capsule) can comprise a single layer coating, e.g., a non-enteric coating such as gelatin or HPMC. For example, enteric coated mini-tablets can be in a gelatin or HPMC capsule.

A therapeutic composition comprising extracellular vesicles from Prevotella histicola bacteria can be formulated as a suspension, e.g., for oral administration or for injection. Administration by injection includes intravenous (IV), intramuscular (IM), and subcutaneous (SC) administration. For a suspension, extracellular vesicles from Prevotella histicola bacteria can be in a buffer, e.g., a pharmaceutically acceptable buffer, e.g., saline or PBS. The suspension can comprise one or more excipients, e.g., pharmaceutically acceptable excipients. The suspension can comprise, e.g., sucrose or glucose. The Prevotella bacteria EVs in the suspension can be isolated extracellular vesicles from Prevotella histicola bacteria. Optionally, the Prevotella histicola bacteria EVs in the suspension can be lyophilized. Optionally, the extracellular vesicles from Prevotella histicola bacteria in the suspension can be gamma irradiated.

### Solutions and Dried Forms

The disclosure provides solutions (for example, liquid mixtures) that comprise Prevotella histicola EVs (for example, Prevotella histicola EVs and/or a combination of EVs described herein). For example, in some embodiments, a solution includes Prevotella histicola EVs and an excipient that comprises a bulking agent. As another example, in some embodiments, a solution includes Prevotella histicola EVs and an excipient that comprises a bulking agent and a lyoprotectant. As another example, in some embodiments, a solution includes Prevotella histicola EVs and an excipient that comprises a lyoprotectant.

For example, in some embodiments, the bulking agent comprises mannitol, sucrose, maltodextrin, dextran, Ficoll, or PVP-K30. In some embodiments, the excipient optionally includes an additional component such as trehalose, mannitol, sucrose, sorbitol, maltodextrin, dextran, poloxamer 188, maltodextrin, PVP-K30, Ficoll, citrate, arginine, and/or hydroxypropyl-β-cyclodextrin. For example, in some embodiments, a solution contains a liquid preparation of EVs and an excipient that comprises a bulking agent, for example, an excipient from a stock of a formula provided in one of Tables A, B, C, D, K, or P. For example, in some embodiments, a solution includes a liquid preparation containing Prevotella histicola EVs (for example, obtained by isolating Prevotella histicola EVs from a bacterial culture (such as the supernatant) or a retentate) and an excipient that comprises a bulking agent, for example, a liquid preparation containing Prevotella histicola EVs is combined with an excipient stock that comprises a bulking agent, for example, an excipient stock of a formula provided in one of Tables A, B, C, D, K, or P, to prepare the solution.

A “dried form” that contains Prevotella histicola extracellular vesicles (EVs) refers to the product resulting from drying a solution that contains Prevotella histicola EVs. In some embodiments, the drying is performed by freeze drying (lyophilization) or spray drying. In some embodiments, the dried form is a powder. As used herein, a powder refers to a type of dried form and includes a lyophilized powder, but includes powders, such as spray-dried powders, obtained by methods such as spray drying.

When freeze drying (lyophilization) is performed, the resulting product is a lyophilate. In some embodiments, the dried form is a lyophilate. As used herein, a lyophilate refers to a type of dried form and includes a lyophilized powder and lyophilized cake. In some embodiments, the lyophilized cake is milled (for example, ground) to produce a lyophilized powder. Milling refers to mechanical size reduction of solids. Grinding is a type of milling, for example, that can be performed on dried forms. See, for example, Seibert et al., “MILLING OPERATIONS IN THE PHARMACEUTICAL INDUSTRY” in Chemical Engineering in the Pharmaceutical Industry: R&D to Manufacturing, Edited by David J. am Ende (2011).

The disclosure also provides dried forms, in some embodiments, such as lyophilates, that comprise Prevotella histicola EVs (for example, Prevotella histicola EVs and/or a combination of EVs described herein), and an excipient. For example, a dried form can include Prevotella histicola EVs and an excipient that comprises a bulking agent. As another example, a dried form can include Prevotella histicola EVs and an excipient that comprises a bulking agent and a lyoprotectant. As another example, a dried form can include Prevotella histicola EVs and an excipient that comprises a lyoprotectant. For example, as described herein, in some embodiments, Prevotella histicola EVs are combined with an excipient that comprises a bulking agent and/or lyoprotectant, for example, to prepare a solution. In some embodiments, the solution is dried. The resulting dried form (for example, lyophilate) contains Prevotella histicola EVs and a component(s) of the excipient, for example, a bulking agent and/or a lyoprotectant (for example, in dried form).

The disclosure also provides dried forms of Prevotella histicola EVs and an excipient. In some embodiments, the dried form is a lyophilate, for example, such as a lyophilized cake or lyophilized powder. In some embodiments, the dried form is a powder, for example, such as a spray-dried powder or lyophilized powder. For example, in some embodiments, the bulking agent comprises mannitol, sucrose, maltodextrin, dextran, Ficoll, or PVP-K30. In some embodiments, the excipient includes an additional component such as trehalose, mannitol, sucrose, sorbitol, dextran, poloxamer 188, maltodextrin, PVP-K30, Ficoll, citrate, arginine, and/or hydroxypropyl-β-cyclodextrin. For example, in some embodiments, a dried form contains Prevotella histicola EVs and an excipient, for example, that comprises a bulking agent, for example, an excipient from a stock of a formula provided in one of Tables A, B, C, D, K, or P. In some embodiments, the dried form has a moisture content below about 6% (or below about 5%) (for example, as determined by Karl Fischer titration). In some embodiments, the dried form has about 10% to about 80% (by weight) of an excipient, for example, an excipient that comprises a bulking agent. In some embodiments, the dried form has about 10% to about 80% (by weight) of an excipient, for example, an excipient from a stock of a formula provided in one of Tables A, B, C, D, K, or P. In some embodiments, the Prevotella histicola EVs comprise about 1% to about 99% of the total solids by weight of the dried form. In some embodiments, the dried form has at least about Prevotella histicola 1 e10 particles per mg of the dried form (for example, as determined by particles per mg, such as by NTA). In some embodiments, the particles of the dried form have a hydrodynamic diameter (Z average, Zₐᵥₑ) of about 130 nm to about 300 nm after resuspension from the dried form (for example, resuspension in deionized water) (for example, as determined by dynamic light scattering).

In some embodiments, the solutions and/or dried form comprise Prevotella histicola EVs substantially or entirely free of whole Prevotella histicola bacteria (for example, live bacteria, killed bacteria, and/or attenuated bacteria). In some embodiments, the solutions and/or dried form comprise both Prevotella histicola EVs and Prevotella histicola whole bacteria (for example, live bacteria, killed bacteria, and/or attenuated bacteria). In some embodiments, the solutions and/or dried form comprise gamma irradiated Prevotella histicola EVs. In some embodiments, the Prevotella histicola EVs are gamma irradiated after the EVs are isolated (for example, prepared).

In some embodiments, to quantify the numbers of Prevotella histicola EVs from bacteria from bacteria) and/or bacteria present in a bacterial sample, electron microscopy (for example, EM of ultrathin frozen sections) is used to visualize the EVs and/or bacteria and count their relative numbers. Alternatively, nanoparticle tracking analysis (NTA), Coulter counting, or dynamic light scattering (DLS) or a combination of these techniques is used. NTA and the Coulter counter count particles and show their sizes. DLS gives the size distribution of particles, but not the concentration. Bacteria frequently have diameters of 1-2 μm (microns). The full range is 0.2-20 μm. Combined results from Coulter counting and NTA can reveal the numbers of bacteria and/or EVs from bacteria in a given sample. Coulter counting reveals the numbers of particles with diameters of 0.7-10 μm. For most bacterial and/or EV samples, the Coulter counter alone can reveal the number of bacteria and/or EVs in a sample. For NTA, a Nanosight instrument can be obtained from Malvern Pananlytical. For example, the NS300 can visualize and measure particles in suspension in the size range 10-2000 nm. NTA allows for counting of the numbers of particles that are, for example, 50-1000 nm in diameter. DLS reveals the distribution of particles of different diameters within an approximate range of 1 nm-3 μm.

In some embodiments, Prevotella histicola EVs are characterized by analytical methods known in the art (for example, Jeppesen, et al. Cell 177:428 (2019)).

In some embodiments, the Prevotella histicola EVs are quantified based on particle count. For example, particle count of an EV preparation can be measured using NTA. For example, particle count of an EV preparation can be measured using NTA with Zetaview.

In some embodiments, the Prevotella histicola EVs are quantified based on the amount of protein, lipid, or carbohydrate. For example, in some embodiments, total protein content of an EV preparation is measured using the Bradford assay or BCA.

In some embodiments, the Prevotella histicola EVs are isolated away from one or more other bacterial components of the source bacteria. In some embodiments, the solution and/or dried form further comprises other bacterial components.

In certain embodiments, the Prevotella histicola EV liquid preparation obtained from the source bacteria may be fractionated into subpopulations based on the physical properties (for example, size, density, protein content, and/or binding affinity) of the subpopulations. One or more of the EV subpopulations (for example, as a liquid preparation) can then be incorporated into the solutions, powders, and/or lyophilates disclosed herein.

In certain aspects, provided herein are solutions and/or dried forms (and therapeutic compositions thereof) comprising Prevotella histicola EVs from bacteria useful for the reduction of inflammatory cytokine expression (e.g., IL-8, IL-6, IL-1β, and/or TNFα expression) and/or for the treatment of bacterial septic shock, cytokine storm and/or viral infection (such as a respiratory viral infection, such as a coronavirus infection (e.g., a MERS (Middle East Respiratory Syndrome) infection, a severe acute respiratory syndrome (SARS) infection, such as a SARS-CoV-2 infection), an influenza infection, and/or a respiratory syncytial virus infection), as well as methods of making and/or identifying such EVs, and methods of using such solutions and/or dried forms (and therapeutic compositions thereof) (for example, for, either alone or in combination with other therapeutics). In some embodiments, the therapeutic compositions comprise both Prevotella histicola EVs, and Prevotella histicola whole bacteria (for example, live bacteria, killed bacteria, and/or attenuated bacteria). In some embodiments, the solutions and/or dried forms comprise EVs from bacteria of one or more (e.g., 1, 2, 3, 4, 5, 6, 7, 8, 9, 10 or more) taxonomic groups (e.g., class, order, family, genus, species or strain). In some embodiments, the solutions and/or dried forms comprise EVs from bacteria of one or more (e.g., 1, 2, 3, 4, 5, 6, 7, 8, 9, 10 or more) bacteria strains or species. In some embodiments, the therapeutic compositions comprise Prevotella histicola EVs in the absence of bacteria (for example, at least about 85%, at least about 90%, at least about 95%, or at least about 99% free of bacteria). In some embodiments, the therapeutic compositions comprise EVs from bacteria of one or more (e.g., 1, 2, 3, 4, 5, 6, 7, 8, 9, 10 or more) taxonomic groups (e.g., class, order, family, genus, species or strain). In some embodiments, the therapeutic compositions comprise EVs from bacteria of one or more (e.g., 1, 2, 3, 4, 5, 6, 7, 8, 9, 10 or more) bacteria strains or species.

In some embodiments, the solution and/or dried form is added to or incorporated into a food product (for example, a food or beverage) such as a health food or beverage, a food or beverage for infants, a food or beverage for pregnant women, athletes, senior citizens or other specified group, a functional food, a beverage, a food or beverage for specified health use, a dietary supplement, a probiotic, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments, the solution and/or dried form is added to a food product or food supplement for animals, including humans. The animals, other than humans, are not particularly limited, and the composition can be used for various livestock, poultry, pets, experimental animals, and the like. Specific examples of the animals include pigs, cattle, horses, sheep, goats, chickens, ducks, ostriches, turkeys, dogs, cats, rabbits, hamsters, mice, rats, monkeys, and the like, but the animals are not limited thereto.

### Therapeutic Compositions

In some embodiments, a solution and/or dried form provided herein is formulated into a therapeutic composition.

In certain embodiments, provided herein are therapeutic compositions comprising a solution and/or dried form described herein. In some embodiments, the therapeutic composition comprises a solution and/or dried form provided herein and a pharmaceutically acceptable carrier. In some embodiments, the therapeutic composition comprises a pharmaceutically acceptable excipient, such as a glidant, lubricant, and/or diluent.

In certain aspects, provided herein are therapeutic compositions comprising EVs from Prevotella histicola bacteria useful for the reduction of inflammatory cytokine expression (e.g., IL-8, IL-6, IL-1β, and/or TNFα expression) and/or for the treatment of bacterial septic shock, cytokine storm and/or viral infection (such as a respiratory viral infection, such as a coronavirus infection (e.g., a MERS (Middle East Respiratory Syndrome) infection, a severe acute respiratory syndrome (SARS) infection, such as a SARS-CoV-2 infection), an influenza infection, and/or a respiratory syncytial virus infection) and/or the treatment of an IL-8, IL-6, IL-1β, and/or TNFα-mediated disease or condition, as well as methods of making and/or identifying such EVs, and methods of using such therapeutic compositions (e.g., for the reduction of inflammatory cytokine expression (e.g., IL-8, IL-6, IL-1β, and/or TNFα expression) and/or for the treatment of bacterial septic shock, cytokine storm and/or viral infection (such as a respiratory viral infection, such as a coronavirus infection (e.g., a MERS (Middle East Respiratory Syndrome) infection, a severe acute respiratory syndrome (SARS) infection, such as a SARS-CoV-2 infection), an influenza infection, and/or a respiratory syncytial virus infection and/or the treatment of an IL-8, IL-6, IL-1β, and/or TNFα-mediated disease or condition), either alone or in combination with other therapeutics). In some embodiments, the therapeutic compositions comprise both Prevotella histicola EVs and whole Prevotella histicola bacteria (e.g., live bacteria, killed bacteria, attenuated bacteria). In some embodiments, the therapeutic compositions comprise Prevotella histicola EVs in the absence of Prevotella histicola bacteria (e.g., at least about 85%, at least about 90%, at least about 95%, or at least about 99% free of bacteria). In some embodiments, the therapeutic compositions comprise EVs and/or bacteria from one or more (e.g., 1, 2, 3, 4, 5, 6, 7, 8, 9, 10 or more) taxonomic groups (e.g., class, order, family, genus, species or strain). In some embodiments, the therapeutic compositions comprise EVs and/or bacteria from one or more of the bacteria strains or species.

In certain aspects, provided are therapeutic compositions for administration to a subject (e.g., human subject). In some embodiments, the therapeutic compositions are combined with additional active and/or inactive materials in order to produce a final product, which may be in single dosage unit or in a multi-dose format. In some embodiments, the therapeutic composition is combined with an adjuvant such as an immuno-adjuvant (e.g., a STING agonist, a TLR agonist, or a NOD agonist).

In some embodiments, the therapeutic composition comprises at least one carbohydrate.

In some embodiments, the therapeutic composition comprises at least one lipid. In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments, the therapeutic composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the therapeutic composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the therapeutic composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the therapeutic composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the therapeutic composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the therapeutic composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the therapeutic composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the therapeutic composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments, the therapeutic composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Dose Forms

In some embodiments, a therapeutic composition comprising a dried form is formulated as a solid dosage form (also referred to as “solid dose form”), for example, for oral administration. In some embodiments, the solid dosage form comprises one or more excipients, for example, pharmaceutically acceptable excipients, in addition to the dried form. The dried form in the solid dosage form contains isolated Prevotella histicola EVs. Optionally, the Prevotella histicola EVs in the solid dosage form are gamma irradiated. In some embodiments, the solid dosage form comprises a tablet, a minitablet, a capsule, or a powder; or a combination of these forms (for example, minitablets comprised in a capsule).

The solid dosage form described herein can be, e.g., a capsule. The solid dosage form described herein can be, e.g., a tablet or a minitablet. Further, a plurality of minitablets can be in (e.g., loaded into) a capsule.

In certain embodiments, the solid dosage form comprises a capsule. In some embodiments, the capsule is a size 00, size 0, size 1, size 2, size 3, size 4, or size 5 capsule. In some embodiments, the capsule is a size 0 capsule. As used herein, the size of the capsule refers to the size of the tablet prior to application of an enteric coating. In some embodiments, the capsule is banded after loading (and prior to enterically coating the capsule). In some embodiments, the capsule is banded with an HPMC-based banding solution.

In some embodiments, the solid dosage form comprises a tablet (>4 mm) (e.g., 5 mm-17 mm). For example, the tablet is a 5 mm, 6 mm, 7 mm, 8 mm, 9 mm, 10 mm, 11 mm, 12 mm, 13 mm, 14 mm, 15 mm, 16 mm, 17 mm, or 18 mm tablet. The size refers to the diameter of the tablet, as is known in the art. As used herein, the size of the tablet refers to the size of the tablet prior to application of an enteric coating.

In some embodiments, the solid dosage form comprises a minitablet. The minitablet can be in the size range of 1 mm-4 mm range. E.g., the minitablet can be a 1 mm minitablet, 1.5 mm minitablet, 2 mm minitablet, 3 mm minitablet, or 4 mm minitablet. The size refers to the diameter of the minitablet, as is known in the art. As used herein, the size of the minitablet refers to the size of the minitablet prior to application of an enteric coating.

The minitablets can be in a capsule. The capsule can be a size 00, size 0, size 1, size 2, size 3, size 4, or size 5 capsule. The capsule that contains the minitablets can comprise hydroxyl propyl methyl cellulose (HPMC) or gelatin. The minitablets can be inside a capsule: the number of minitablets inside a capsule will depend on the size of the capsule and the size of the minitablets. As an example, a size 0 capsule can contain 31-35 (an average of 33) minitablets that are 3 mm minitablets. In some embodiments, the capsule is banded after loading. In some embodiments, the capsule is banded with an HPMC-based banding solution.

A therapeutic composition comprising a solution and/or powder (e.g., that comprises EVs and a bulking agent) can be formulated as a suspension (e.g., a powder can be reconstituted; a solution can be diluted), e.g., for oral administration or for injection. Administration by injection includes intravenous (IV), intramuscular (IM), and subcutaneous (SC) administration. For a suspension, EVs can be in a buffer, e.g., a pharmaceutically acceptable buffer, e.g., saline or PBS. The suspension can comprise one or more excipients, e.g., pharmaceutically acceptable excipients. The suspension can comprise, e.g., sucrose or glucose. The EVs in the solution or powder (e.g., that comprises EVs and a bulking agent) can be isolated EVs. Optionally, the EVs in the suspension can be gamma irradiated.

### Coating

A solid dosage form (e.g., capsule, tablet or minitablet) described herein can be enterically coated, e.g., with one enteric coating layer or with two layers of enteric coating, e.g., an inner enteric coating and an outer enteric coating. The inner enteric coating and outer enteric coating are not identical (e.g., the inner enteric coating and outer enteric coating do not contain the same components in the same amounts). The enteric coating allows for release of the therapeutic agent (such as Prevotella histicola EVs, dried forms, and/or solid dosage forms thereof), e.g., in the small intestine.

Release of the therapeutic agent in the small intestine allows the therapeutic agent to target and affect cells (e.g., epithelial cells and/or immune cells) located at these specific locations, e.g., which can cause a local effect in the gastrointestinal tract and/or cause a systemic effect (e.g., an effect outside of the gastrointestinal tract).

EUDRAGIT is the brand name for a diverse range of polymethacrylate-based copolymers. It includes anionic, cationic, and neutral copolymers based on methacrylic acid and methacrylic/acrylic esters or their derivatives.

Examples of other materials that can be used in the enteric coating (e.g., the one enteric coating or the inner enteric coating and/or the outer enteric coating) include cellulose acetate phthalate (CAP), cellulose acetate trimellitate (CAT), poly(vinyl acetate phthalate) (PVAP), hydroxypropyl methylcellulose phthalate (HPMCP), fatty acids, waxes, shellac (esters of aleurtic acid), plastics, plant fibers, zein, Aqua-Zein® (an aqueous zein formulation containing no alcohol), amylose starch, starch derivatives, dextrins, methyl acrylate-methacrylic acid copolymers, cellulose acetate succinate, hydroxypropyl methyl cellulose acetate succinate (hypromellose acetate succinate), methyl methacrylate-methacrylic acid copolymers, and/or sodium alginate.

The enteric coating (e.g., the one enteric coating or the inner enteric coating and/or the outer enteric coating) can include a methacrylic acid ethyl acrylate (MAE) copolymer (1:1).

The one enteric coating can include methacrylic acid ethyl acrylate (MAE) copolymer (1:1) (such as Kollicoat MAE 100P).

The one enteric coating can include a Eudragit copolymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

Other examples of materials that can be used in the enteric coating (e.g., the one enteric coating or the inner enteric coating and/or the outer enteric coating) include those described in, e.g., U.S. Pat. Nos. 6,312,728; 6,623,759; 4,775,536; 5,047,258; 5,292,522; 6,555,124; 6,638,534; U.S. 2006/0210631; U.S. 2008/200482; U.S. 2005/0271778; U.S. 2004/0028737; WO 2005/044240.

See also, e.g., U.S. Pat. No. 9,233,074, which provides pH dependent, enteric polymers that can be used with the solid dosage forms provided herein, including methacrylic acid copolymers, polyvinylacetate phthalate, hydroxypropylmethyl cellulose acetate succinate, hydroxypropylmethyl cellulose phthalate and cellulose acetate phthalate; suitable methacrylic acid copolymers include: poly(methacrylic acid, methyl methacrylate) 1:1 sold, for example, under the Eudragit L100 trade name; poly(methacrylic acid, ethyl acrylate) 1:1 sold, for example, under the Eudragit L100-55 trade name; partially-neutralized poly(methacrylic acid, ethyl acrylate) 1:1 sold, for example, under the Kollicoat MAE-100P trade name; and poly(methacrylic acid, methyl methacrylate) 1:2 sold, for example, under the Eudragit S100 trade name.

The solid dose form (e.g., a capsule) can comprise a single layer coating, e.g., a non-enteric coating such as HPMC (hydroxyl propyl methyl cellulose) or gelatin.

### Method of Making Solutions and Dried Forms

The disclosure also provides methods of preparing solutions of Prevotella histicola EVs and an excipient that comprises a bulking agent. For example, in some embodiments, the bulking agent comprises mannitol, sucrose, polyethylene glycol (PEG, such as PEG 6000), cyclodextrin, maltodextrin, dextran, Ficoll, or PVP-K30. In some embodiments, the excipient comprises a lyoprotectant. In some embodiments, the excipient optionally includes an additional component such as trehalose, mannitol, sucrose, sorbitol, dextran, poloxamer 188, maltodextrin, PVP-K30, Ficoll, citrate, arginine, and/or hydroxypropyl-β-cyclodextrin. For example, in some embodiments, a liquid preparation of Prevotella histicola EVs and an excipient that comprises a bulking agent are combined to prepare a solution. For example, in some embodiments, a liquid preparation of Prevotella histicola EVs (for example, obtained by isolating EVs from a bacterial culture (such as a supernatant or a retentate) and an excipient that comprises a bulking agent, for example, an excipient stock of a formula provided in one of Tables A, B, C, D, K, or P, are combined to prepare a solution. For example, in some embodiments, a liquid preparation containing Prevotella histicola EVs (for example, obtained by isolating EVs from a bacterial culture (such as a supernatant or a retentate) and an excipient that comprises a bulking agent are combined, for example, a liquid preparation containing Prevotella histicola EVs (for example, obtained by isolating EVs from a bacterial culture (such as a supernatant or a retentate) or a retentate) are combined with an excipient that comprises a bulking agent, for example, such as mannitol or an excipient of an excipient stock of a formula provided in one of Tables A, B, C, D, K, or P, to prepare the solution.

The disclosure also provides methods of preparing dried forms of Prevotella histicola EVs. For example, in some embodiments, the method is used to prepare a lyophilate such as a lyophilized powder and/or a lyophilized cake. For example, in some embodiments, the method is used to prepare a powder such as a lyophilized powder and/or a spray-dried powder. In some embodiments, the excipient comprises a bulking agent. For example, in some embodiments, the bulking agent comprises mannitol, sucrose, polyethylene glycol (PEG, such as PEG 6000), cyclodextrin, maltodextrin, dextran, Ficoll, or PVP-K30. In some embodiments, the excipient comprises a lyoprotectant. In some embodiments, the excipient optionally includes an additional component such as trehalose, mannitol, sucrose, sorbitol, dextran, poloxamer 188, maltodextrin, PVP-K30, Ficoll, citrate, arginine, and/or hydroxypropyl-β-cyclodextrin. For example, in some embodiments, a liquid preparation containing Prevotella histicola EVs (for example, obtained by isolating EVs from a bacterial culture (such as a supernatant or a retentate) is be combined with an excipient that comprises a bulking agent, such as mannitol or an excipient of an excipient stock of a formula provided in one of Tables A, B, C, D, K, or P; and dried (for example, by lyophilization or spray drying) to thereby prepare a dried form. In some embodiments, the dried form has a moisture content below about 6%, below about 5%, below about 4%, between about 0.5% to about 5%, between about 1% to about 5%, between about 1% to about 4%, between about 1.5% to about 4%, between about 2% and about 4%, or between about 2% to about 3%, (for example, as determined by Karl Fischer titration). In some embodiments, the dried form has about 10% to about 80% (by weight) of an excipient, for example, an excipient that comprises a bulking agent. In some embodiments, the dried form has about 10% to about 80% (by weight) of an excipient, for example, an excipient from a stock of a formula provided in one of Tables A, B, C, D, K, or P. In some embodiments, the Prevotella histicola EVs comprise about 1% to about 99% of the total solids by weight of the dried form. In some embodiments, the dried form has at least about 1 e10 Prevotella histicola particles per mg of the dried form (for example, as determined by particles per mg, such as by NTA). In some embodiments, the particles in the dried form have a hydrodynamic diameter (Z average, Zₐᵥₑ) of about 130 nm to about 300 nm after resuspension from the dried form (for example, resuspension in deionized water) (for example, as determined by dynamic light scattering).

In some embodiments, the dried form is a lyophilate. In some embodiments, the lyophilate is a lyophilized powder or a lyophilized cake. In some embodiments, the dried form is a powder. In some embodiments, the powder is a lyophilized powder or a spray-dried powder.

In some embodiments, a method of preparing a solution that comprises extracellular vesicles (EVs) from Prevotella histicola bacteria includes: combining a liquid preparation that comprises EVs from Prevotella histicola bacteria with an excipient that comprises a bulking agent, thereby preparing the solution.

In some embodiments, a method of preparing a solution that comprises extracellular vesicles (EVs) from Prevotella histicola bacteria includes: combining a liquid preparation that comprises EVs from Prevotella histicola bacteria with an excipient that comprises a bulking agent and a lyoprotectant, thereby preparing the solution.

In some embodiments, a method of preparing a solution that comprises extracellular vesicles (EVs) from Prevotella histicola bacteria includes: combining a liquid preparation that comprises EVs from Prevotella histicola bacteria with an excipient that comprises a lyoprotectant, thereby preparing the solution.

In some embodiments, a method of preparing a solution that comprises extracellular vesicles (EVs) from Prevotella histicola bacteria includes: combining a liquid preparation that comprises EVs from Prevotella histicola bacteria with a stock comprising one or more excipients, wherein the stock comprises a formula provided in Table A, B, C, D, K, or P, thereby preparing a solution.

In some embodiments, the disclosure provides a solution prepared by a method described herein.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
drying the solution to prepare a cake, and
milling (for example, grinding the cake, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
drying the solution to prepare a cake, and
milling (for example, grinding) the cake, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
drying the solution to prepare a cake, and
milling (for example, grinding) the cake, thereby preparing the dried form.

In some embodiments, the drying comprises lyophilization.

In some embodiments, the drying comprises spray drying.

In some embodiments, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
drying the solution to prepare a cake, and
milling (for example, grinding) the cake, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
drying the solution to prepare a cake, and
milling (for example, grinding) the cake, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
drying the solution to prepare a cake, and
milling (for example, grinding) the cake, thereby preparing the powder.

In some embodiments, the drying comprises lyophilization.

In some embodiments, the drying comprises spray drying.

In some embodiments, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
spray drying the solution, thereby preparing the spray-dried powder.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
spray drying the solution, thereby preparing the spray-dried powder.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
spray drying the solution, thereby preparing the spray-dried powder.

In some embodiments, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
freeze drying (lyophilizing) the solution to prepare a cake, and
milling (for example, grinding) the cake, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
freeze drying (lyophilizing) the solution to prepare a cake, and
milling (for example, grinding) the cake, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
freeze drying (lyophilizing) the solution to prepare a cake, and
milling (for example, grinding) the cake, thereby preparing the lyophilate.

In some embodiments, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
freeze drying (lyophilizing) the solution to prepare a cake, and
milling (for example, grinding) the cake, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
freeze drying (lyophilizing) the solution to prepare a cake, and
milling (for example, grinding) the cake, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
freeze drying (lyophilizing) the solution to prepare a cake, and
milling (for example, grinding) the cake, thereby preparing the lyophilized powder.

In some embodiments, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilized cake.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilized cake.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilized cake.

In some embodiments, the disclosure provides a lyophilized cake prepared by a method described herein.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution.

In some embodiments, the disclosure provides a solution prepared by a method described herein.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
drying the solution to prepare a cake, and
milling (for example, grinding) the cake, thereby preparing the dried form.

In some embodiments, the drying comprises lyophilization.

In some embodiments, the drying comprises spray drying.

In some embodiments, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
drying the solution to prepare a cake, and
milling (for example, grinding) the cake, thereby preparing the powder.

In some embodiments, the drying comprises lyophilization.

In some embodiments, the drying comprises spray drying.

In some embodiments, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
spray drying the solution, thereby preparing the spray-dried powder.

In some embodiments, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
freeze drying (lyophilizing) the solution to prepare a cake, and
milling (for example, grinding) the cake, thereby preparing the lyophilate.

In some embodiments, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
freeze drying (lyophilizing) the solution to prepare a cake, and
milling (for example, grinding) the cake, thereby preparing the lyophilized powder.

In some embodiments, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing a lyophilized cake.

In some embodiments, the disclosure provides a lyophilized cake prepared by a method described herein.

### Method of Preparing Therapeutic Compositions

The disclosure also provides methods of preparing therapeutic compositions. In some embodiments, the method includes combining a solution or dried form described herein with a pharmaceutically acceptable excipient, such as a glidant, lubricant, and/or diluent, thereby preparing a therapeutic composition.

The disclosure also provides methods of preparing therapeutic compositions, such as solid dosage forms, that contain a dried form described herein. In some embodiments, the solid dosage form is a capsule, tablet, or minitablet.

The disclosure also provides methods of making a solid dosage form (for example, for oral administration) (for example, for pharmaceutical use) that comprises a dried form. In some embodiments, the dried form comprises Prevotella histicola extracellular vesicles (EVs) and an excipient that comprises a bulking agent. In some embodiments, the dried form comprises Prevotella histicola extracellular vesicles (EVs) and an excipient that comprises a lyoprotectant. In some embodiments, the dried form comprises Prevotella histicola extracellular vesicles (EVs) and an excipient that comprises a bulking agent and a lyoprotectant. In some embodiments, the dried form also contains one or more additional components. In some embodiments, the dried form is combined with one or more pharmaceutically acceptable excipients. In some embodiments, the solid dosage form is enterically coated, for example, with a coating described herein.

In some aspects, a method of making the solid dosage form includes:
loading a dried form that comprises Prevotella histicola extracellular vesicles (EVs) into a capsule, thereby preparing a capsule, and thereby preparing the solid dosage form;
optionally combining the dried form with a pharmaceutically acceptable excipient prior to loading into the capsule; and/or
optionally banding the capsule after loading the capsule (for example, optionally banding the capsule after loading the capsule).

In some aspects, a method of making the solid dosage form includes: compressing a dried form that comprises Prevotella histicola extracellular vesicles (EVs) described herein into a minitablet, thereby preparing a minitablet and thereby preparing the solid dosage form;
optionally combining the dried form with a pharmaceutically acceptable excipient prior to compressing;
optionally filling a capsule with a plurality of enterically coated minitablets.

In some aspects, a method of making the solid dosage form includes:
compressing a dried form that comprises Prevotella histicola extracellular vesicles (EVs) described herein into a tablet, thereby preparing a tablet, and thereby preparing the solid dosage form;
optionally combining the dried form with a pharmaceutically acceptable excipient prior to compressing.

In certain embodiments, the method comprises performing wet granulation on a powder prior to combining the powder and one or more (for example, one, two or three) excipients into a therapeutic composition, such as a solid dosage form. In some embodiments, the wet granulation comprises (i) mixing the powder with a granulating fluid (for example, water, ethanol, or isopropanol, alone or in combination). In some embodiments, the wet granulation comprises mixing the powder with water. In some embodiments, the wet granulation comprises (ii) drying mixed powder and granulating fluid (for example, drying on a fluid bed dryer). In some embodiments, the wet granulation comprises (iii) milling (for example, grinding) the dried powder and granulating fluid. The milled (for example, ground) powder and granulating fluid are then combined with the one or more (for example, one, two or three) excipients to prepare a therapeutic composition, such as a solid dosage form. In some embodiments, the powder is a lyophilized powder. In some embodiments, the powder is a spray-dried powder.

In some embodiments, a dried form described herein is reconstituted in a liquid (for example, a buffer, juice, or water) to prepare a therapeutic composition.

In some embodiments, a solution is resuspended (for example, diluted) in a liquid (for example, a buffer, juice, or water) to prepare a therapeutic composition.

In some embodiments, a therapeutic composition comprising a dried form described herein is reconstituted in a liquid (for example, a buffer, juice, or water) to prepare a suspension.

In some embodiments, a therapeutic composition comprising a solution is resuspended (for example, diluted) in a liquid (for example, a buffer, juice, or water) to prepare a suspension.

### Gamma-Irradiation

Dried forms such as powders (e.g., of EVs from Prevotella histicola bacteria) can be gamma-irradiated at 17.5 kGy radiation unit at ambient temperature.

Frozen biomasses (e.g., of EVs from Prevotella histicola bacteria) can be gamma-irradiated at 25 kGy radiation unit in the presence of dry ice.

### Additional Therapeutic Agents

In certain aspects, the methods provided herein include the administration to a subject of a therapeutic composition described herein either alone or in combination with an additional therapeutic agent. In some embodiments, the additional therapeutic agent is an immunosuppressant, an anti-inflammatory agent, and/or a steroid.

In some embodiments, the therapeutic composition comprising EVs from Prevotella histicola bacteria is administered to the subject before the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, the therapeutic composition comprising EVs from Prevotella histicola bacteria is administered to the subject after the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the therapeutic composition comprising EVs from Prevotella histicola bacteria and the additional therapeutic agent are administered to the subject simultaneously or nearly simultaneously (e.g., administrations occur within an hour of each other).

In some embodiments, an antibiotic is administered to the subject before the therapeutic composition comprising EVs from Prevotella histicola bacteria is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, an antibiotic is administered to the subject after therapeutic composition comprising EVs from Prevotella histicola bacteria is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the therapeutic composition comprising EVs from Prevotella histicola bacteria and the antibiotic are administered to the subject simultaneously or nearly simultaneously (e.g., administrations occur within an hour of each other).

In some embodiments, the methods provided herein include the administration of a therapeutic composition described herein in combination with one or more additional therapeutic agents. In some embodiments, the methods disclosed herein include the administration of two therapeutic agents.

In some embodiments, the therapeutic agent is an antibiotic. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (e.g., Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86(2): 156-167). In certain embodiments, antibiotics can be used to selectively target bacteria of a specific niche. In other embodiments, antibiotics are administered after the therapeutic composition comprising EVs from Prevotella histicola bacteria. In some embodiments, antibiotics are administered before therapeutic composition comprising EVs from Prevotella histicola bacteria.

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective, e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin P1, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JHl 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

In some embodiments, the additional therapeutic agent is an immunosuppressive agent, a DMARD, a pain-control drug, a steroid, a non-steroidal anti-inflammatory drug (NSAID), or a cytokine antagonist, and combinations thereof. Representative agents include, but are not limited to, cyclosporin, retinoids, corticosteroids, propionic acid derivative, acetic acid derivative, enolic acid derivatives, fenamic acid derivatives, Cox-2 inhibitors, lumiracoxib, ibuprophen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetominophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs (e.g., hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists (e.g., TNF alpha antagonists or TNF alpha receptor antagonists), e.g., ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra/Actemra®), integrin antagonists (TYSABRI® (natalizumab)), IL-1 antagonists (ACZ885 (Ilaris)), Anakinra (Kineret®)), CD4 antagonists, IL-23 antagonists, IL-20 antagonists, IL-6 antagonists, BLyS antagonists (e.g., Atacicept, Benlysta®/LymphoStat-B® (belimumab)), p38 Inhibitors, CD20 antagonists (Ocrelizumab, Ofatumumab (Arzerra®)), interferon gamma antagonists (Fontolizumab), prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin+Viusid, TwHF, Methoxsalen, Vitamin D—ergocalciferol, Milnacipran, Paclitaxel, rosig tazone, Tacrolimus (Prograf®), RADOOl, rapamune, rapamycin, fostamatinib, Fentanyl, XOMA 052, Fostamatinib disodium, rosightazone, Curcumin (Longvida™), Rosuvastatin, Maraviroc, ramipnl, Milnacipran, Cobiprostone, somatropin, tgAAC94 gene therapy vector, MK0359, GW856553, esomeprazole, everolimus, trastuzumab, JAK1 and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists (Tysarbri® (natalizumab)), VGEF antagnosits, CXCL antagonists, MMP antagonists, defensin antagonists, IL-1 antagonists (including IL-1 beta antagonsits), and IL-23 antagonists (e.g., receptor decoys, antagonistic antibodies, etc.).

In some embodiments, the additional therapeutic agent is an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

### Additional Therapy

In some embodiments, an additional therapy is administered to the subject. In some embodiments, the additional therapy comprises an antiviral medication. In some embodiments, the additional therapy comprises an antiviral medication such as ribavirin, neuraminidase inhibitor, protease inhibitor, recombinant interferons, antibodies, oseltamivir, zanamivir, peramivir or baloxavir marboxil. In some embodiments, the additional therapy comprises hydroxychloroquine and/or chloroquine. In some embodiments, the additional therapy comprises remdesivir. In some embodiments, the additional therapy comprises plasma from a subject who has recovered from infection by the same virus that is infecting the subject (e.g., plasma from a subject who has recovered from SARS-CoV-2 infection) (e.g., convalescent plasma therapy).

In some embodiments, the additional therapy comprises an anti-inflammatory agent such as an NSAID or an anti-inflammatory steroid. In some embodiments, the additional therapy comprises a corticosteroid such as dexamethasone, prednisone, methylprednisolone, or hydrocortisone. In some embodiments, the additional therapy comprises dexamethasone.

In some embodiments, the additional therapy comprises an antibody specific for IL-6 and/or the IL-6 receptor. In some embodiments, the additional therapy comprises tocilizumab (Actemra®). In some embodiments, the additional therapy comprises sarilumab (Kevzara®).

In some embodiments, the additional therapy can comprise an anti-viral therapy. For example, the anti-viral therapy can comprise a nucleotide analog, such as remdesivir, galidesivir or clevudine; a viral RNA polymerase inhibitor such as favipiravir or galidesivir; a protease inhibitor such as ritonavir, darunavir, or danoprevir; an inhibitor of viral membrane fusion such as umifenovir; and/or anti-SARS-CoV-2 plasma.

In some embodiments, the additional therapy can comprise an anti-inflammatory therapy. For example, the anti-inflammatory therapy can comprise a corticosteroid; sirolimus; anakinra; filamod; or an antibody. In some embodiments, the antibody can comprise a GMSF inhibitor, such as lenzilumab or gimsilumab; an anti-IL1 beta inhibitor such as canakinumab; an IL-6 inhibitor such as tocilizumab or siltuximab; an IL-6R inhibitor such as sarilumab; and/or a CCR5 antagonist such as leronlimab.

In some embodiments, the additional therapy can comprise a JAK inhibitor such as baricitinib, ruxolitinib, tofacitinib, and/or pacritinib. In some embodiments, the additional therapy can comprise baricitinib. In some embodiments, the additional therapy can comprise baricitinib in combination with remdesivir.

In some embodiments, the additional therapy can comprise a TLR7 agonist such as imiquimod or reisquimod.

In some embodiments, the additional therapy can comprise a cell based therapy. For example, the cell based therapy can comprise Remestemcel-L; bone marrow stem cell therapy, such as MultiStem or Bm-Allo-MSC; mesenchymal stromal cells; and/or adiopose derived mesenchymal stem cells such as AstroStem.

In some embodiments, the additional therapy can comprise an ACE receptor inhibitor. In some embodiments, the additional therapy can comprise an angiotensin-converting enzyme (ACE) inhibitor. In some embodiments, the additional therapy can comprise an angiotensin-converting enzyme 2 (ACE2) inhibitor.

In some embodiments, the additional therapy can comprise a regulator of the Sigma 1 and/or Sigma 2 receptor.

In some embodiments, the additional therapy can comprise IFN-β1a (e.g., by inhalation). In some embodiments, the additional therapy can comprise SNG001 (IFN-β1a for nebulisation).

In some embodiments, the additional therapy can comprise a monoclonal antibody treatment. In some embodiments, the additional therapy can comprise a monoclonal antibody treatment such as bamlanivimab, casirivimab, or imdevimab, or a combination thereof, e.g., a combination of casirivimab and imdevimab. In some embodiments, the additional therapy can comprise a monoclonal antibody treatment such as bamlanivimab or etesevimab, or a combination of bamlanivimab or etesevimab.

In some embodiments, the additional therapy can comprise budesonide, e.g., inhaled budesonide.

In some embodiments, the additional therapy can comprise an anticoagulation drug, such as heparin or enoxaparin (e.g., a low-dose thereof).

In some embodiments, the additional therapy can comprise vitamin D.

In some embodiments, the additional therapy can comprise plitidepsin (also referred to as dehydrodidemnin B) (e.g., marketed as Aplidin).

In some embodiments, the additional therapy can comprise ivermectin.

### Administration

In certain aspects, provided herein is a method of delivering a therapeutic composition described herein (e.g., a therapeutic composition EVs from Prevotella histicola bacteria to a subject. In some embodiments of the methods provided herein, the therapeutic composition is administered in conjunction with the administration of an additional therapeutic agent or additional therapy. In some embodiments, the therapeutic composition comprises EVs from Prevotella histicola bacteria co-formulated with the additional therapeutic agent or additional therapy. In some embodiments, the therapeutic composition comprising EVs from Prevotella histicola bacteria is co-administered with the additional therapeutic agent or additional therapy. In some embodiments, the additional therapeutic agent or additional therapy is administered to the subject before administration of the therapeutic composition that comprises EVs from Prevotella histicola bacteria (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent or additional therapy is administered to the subject after administration of the therapeutic composition that comprises EVs from Prevotella histicola bacteria (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the pharmaceutical composition that comprises EVs from Prevotella histicola bacteria and the additional therapeutic agent or additional therapy. In some embodiments, different modes of delivery are used to administer the pharmaceutical composition that comprises EVs from Prevotella histicola bacteria and the additional therapeutic agent or additional therapy. For example, in some embodiments the therapeutic composition that comprises EVs from Prevotella histicola bacteria is administered orally while the additional therapeutic agent or additional therapy is administered via injection (e.g., an intravenous and/or intramuscular injection).

In certain aspects, provided herein is a method of delivering a therapeutic composition described herein to a subject. In some embodiments, the subject is a mammal. In some embodiments, the subject is a human.

In some embodiments, the therapeutic composition described herein is administered once a day. In some embodiments, the therapeutic composition described herein is administered twice a day. In some embodiments, the therapeutic composition described herein is formulated for a daily dose. In some embodiments, the therapeutic composition described herein is formulated for twice a day dose, wherein each dose is half of the daily dose.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, for example, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a therapeutic composition that comprises EVs from Prevotella histicola bacteria described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent disease (e.g., autoimmune disease, inflammatory disease, or metabolic disease), delay its onset, or slow or stop its progression, or relieve one or more symptoms of the disease. One skilled in the art will recognize that dosage will depend upon a variety of factors including the strength of the particular agent (e.g., therapeutic agent) employed, as well as the age, species, condition, and body weight of the subject. The size of the dose will also be determined by the route, timing, and frequency of administration as well as the existence, nature, and extent of any adverse side-effects that might accompany the administration of a particular therapeutic agent and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the therapeutic agents used in accordance with the as set forth herein vary depending on the active agent, the age, weight, and clinical condition of the recipient patient, and the experience and judgment of the clinician or practitioner administering the therapy, among other factors affecting the selected dosage. As another example, the dose should be sufficient to result in slowing of progression of the disease for which the subject is being treated, and, in some embodiments, amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a therapeutic composition, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month.

In some embodiments, the delivery of an additional therapeutic agent or additional therapy in combination with the therapeutic composition described herein reduces the adverse effects and/or improves the efficacy of the additional therapeutic agent or additional therapy.

The effective dose of an additional therapeutic agent or additional therapy described herein is the amount of the additional therapeutic agent or additional therapy that is effective to achieve the desired therapeutic response for a particular subject, composition, and mode of administration, with the least toxicity to the subject. The effective dosage level can be identified using the methods described herein and will depend upon a variety of pharmacokinetic factors including the activity of the particular compositions or agents administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts. In general, an effective dose of an additional therapeutic agent or additional therapy will be the amount of the additional therapeutic agent or additional therapy which is the lowest dose effective to produce a therapeutic effect. Such an effective dose will generally depend upon the factors described above.

The toxicity of an additional therapeutic agent or additional therapy is the level of adverse effects experienced by the subject during and following treatment. Adverse events associated with additional therapeutic agent/therapy toxicity can include, but are not limited to, abdominal pain, acid indigestion, acid reflux, allergic reactions, alopecia, anaphylasix, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heartbeat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

In some embodiments, the therapeutic composition is administered orally. In some embodiments, the administration to the subject for a single day followed by a washout period before the next dose. In some embodiments, the washout period is at least 12 hours, 24 hours, 36 hours, 48 hours, 50 hours, 60 hours, or 72 hours.

In some embodiments, the therapeutic composition is administered after the washout period once daily for 14 days, 15 days, 16 days, 17 days, 18 days, 19 days, 20 days, 21 days, 22 days, 23 days, 24 days, 25 days, 26 days, 27 days, or 28 days.

In some embodiments, the therapeutic composition is administered after the washout period twice daily for 14 days, 15 days, 16 days, 17 days, 18 days, 19 days, 20 days, 21 days, 22 days, 23 days, 24 days, 25 days, 26 days, 27 days, or 28 days.

In some embodiments, the therapeutic composition is administered for 14 days, 15 days, 16 days, 17 days, 18 days, 19 days, 20 days, 21 days, 22 days, 23 days, 24 days, 25 days, 26 days, 27 days, or 28 days. In some embodiments, the therapeutic composition is administered for 14 days. In some embodiments, the therapeutic composition is administered for 21 days.

In some embodiments, the therapeutic composition is administered twice daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In some embodiments, the therapeutic composition is administered twice daily for 14 days. In some embodiments, the therapeutic 1 composition is administered twice daily for 21 days.

In some embodiments, the therapeutic composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In some embodiments, the therapeutic composition is administered once daily for 14 days. In some embodiments, the therapeutic composition is administered once daily for 21 days.

In some embodiments, the therapeutic composition is administered twice daily for three days and then once daily for the remainder of the treatment (e.g., until day 14).

In some embodiments, the therapeutic composition is formulated as a capsule (e.g., containing mini-tablets or powder) or a tablet. In some embodiments, the therapeutic composition comprises an enteric coating or micro encapsulation. In some embodiments, the therapeutic composition is formulated as a tablet. In some embodiments, the tablet is an enteric coated tablet. In some embodiments, the therapeutic composition is formulated as a capsule. In some embodiments, the capsule is an enteric coated capsule. In some embodiments, the capsule is an HPMC capsule, e.g., that is further enteric coated. In some embodiments, the capsule is a gelatin capsule, e.g., that is further enteric coated.

In some embodiments of the methods provided herein, the therapeutic composition is administered in conjunction with the administration of an additional therapeutic or an additional therapy. In some embodiments, the therapeutic composition comprises extracellular vesicles from Prevotella histicola bacteria co-formulated with the additional therapeutic or an additional therapy. In some embodiments, the therapeutic composition is co-administered with the additional therapeutic or an additional therapy. In some embodiments, the additional therapeutic agent or additional therapy is administered to the subject before administration of the therapeutic composition (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent or additional therapy is administered to the subject after administration of the therapeutic composition (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the therapeutic composition and the additional therapeutic agent or additional therapy. In some embodiments different modes of delivery are used to administer the therapeutic composition and the additional therapeutic agent or additional therapy. For example, in some embodiments the therapeutic composition is administered orally while the additional therapeutic agent or additional therapy is administered via injection (e.g., an intravenous, and/or intramuscular injection).

In some embodiments, the therapeutic composition is administered orally. In some embodiments, the administration to the subject for a single day followed by an interval period before the next dose. In some embodiments, the interval period is at least 3 days, 4 days, 5 days, 6 days, or 7 days.

In some embodiments, the therapeutic composition is administered after the interval period once daily for 14 days, 15 days, 16 days, 17 days, 18 days, 19 days, 20 days, 21 days, 22 days, 23 days, 24 days, 25 days, 26 days, 27 days, or 28 days.

In some embodiments, the therapeutic composition is formulated as a capsule or a tablet. In some embodiments, the therapeutic composition comprises an enteric coating or micro encapsulation. In some embodiments, the capsule is an enteric coated capsule.

In some embodiments, the subject is a mammal. In some embodiments, the subject is a human.

In certain embodiments, the therapeutic compositions and dosage forms, described herein can be administered in conjunction with any other conventional treatment. These treatments may be applied as necessary and/or as indicated and may occur before, concurrent with or after administration of the therapeutic compositions, dosage forms, and kits described herein.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, and other compounds such as drugs being administered concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow, and replicate. The dose of the therapeutic compositions described herein may be appropriately set or adjusted in accordance with the dosage form, the route of administration, the degree or stage of a target disease, and the like.

In some embodiments, the dose administered to a subject is sufficient to delay onset of disease onset, or slow or stop its progression. One skilled in the art will recognize that dosage will depend upon a variety of factors including the strength of the particular compound employed, as well as the age, species, condition, and body weight of the subject. The size of the dose will also be determined by the route, timing, and frequency of administration as well as the existence, nature, and extent of any adverse side-effects that might accompany the administration of a particular compound and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the active agents used in as set forth herein vary depending on the active agent, the age, weight, and clinical condition of the recipient patient, and the experience and judgment of the clinician or practitioner administering the therapy, among other factors affecting the selected dosage.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a therapeutic composition, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response and/or the time period for a subject to clear the bacteria from normal tissue. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the bacteria from normal tissue; for example, the time period can be more than the time period for a subject to clear the bacteria from normal tissue, such as more than about a day, more than about two days, more than about three days, more than about five days, or more than about a week.

In some embodiments, the delivery of an additional therapeutic or an additional therapy in combination with the therapeutic composition described herein reduces the adverse effects and/or improves the efficacy of the additional therapeutic or an additional therapy.

The effective dose of an additional therapeutic or an additional therapy described herein is the amount of the additional therapeutic agent or an additional therapy that is effective to achieve the desired therapeutic response for a particular patient, composition, and mode of administration, with the least toxicity to the patient. The effective dosage level can be identified using the methods described herein and will depend upon a variety of pharmacokinetic factors including the activity of the particular compositions administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. In general, an effective dose of an additional therapy will be the amount of the therapeutic agent which is the lowest dose effective to produce a therapeutic effect. Such an effective dose will generally depend upon the factors described above.

The toxicity of an additional therapy or an additional therapy is the level of adverse effects experienced by the subject during and following treatment. Adverse events associated with additional therapy toxicity include, but are not limited to, abdominal pain, acid indigestion, acid reflux, allergic reactions, alopecia, anaphylaxis, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heartbeat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

In certain embodiments, the therapeutic effects of these orally delivered medicines (e.g., pharmaceutical compositions) come from their action on pattern recognition receptors on immune cells in the lining of the small intestine. These cells, in turn, modulate immune cells circulating throughout the body. The medicines are microbes, but do not target the microbiome. In some embodiments, the microbes do not colonize or persist in the gut and do not modify the colonic microbiome. In some embodiments, they are gut-restricted. In some embodiments, the therapeutic effects of these orally delivered medicines are determined by examining for a biomarker measuring reaction of host (person) to infection (i.e., cytokine response, T cells and T cell ratios); an effect on infection itself (like virus measurement in sputum or swabs); or a clinical endpoint (like mortality or chest x-ray, clearance of virus).

In certain embodiments, the methods provided herein result in change (e.g., an increase or a decrease) in serum and/or expression levels of one or more cytokines (or one or more cellular factors) after the subject is treated according to a method provided herein for a set time interval as compared to before treatment and/or at the onset of treatment. In certain embodiments, the one or more cytokines (or one or more cellular factors) include TNF-α, IL-1β, IL-2, IL-6, IL-7, IL-1β, IP10, MCP1, sIL-2R, IL-8, IL-1Ra, IL-2Ra, IL-18, HGF, MCP-1, MCP-3, MIG, M-CSF, GM-CSF, G-CSF, MIG-1α, and/or macrophage inflammatory protein (MIP)-1alpha (MIP1α). In certain embodiments, the one or more cytokines include TNF-α, IL-1β, IL-6, and/or IL-8. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days. The levels of the one or more cytokines can be determined, e.g., by ex vivo LPS stimulation of whole blood samples obtained from a subject, e.g., as described herein.

In certain embodiments, the methods provided herein result in change (e.g., an increase or a decrease) in serum and/or expression levels C-reactive Protein (CRP) after the subject is treated according to a method provided herein for a set time interval as compared to before treatment and/or at the onset of treatment. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days.

In certain embodiments, the methods provided herein result in change (e.g., an increase or a decrease) in serum T cell count (e.g., CD4⁺ T cell count and/or CD8⁺ T cell count) after the subject is treated according to a method provided herein for a set time interval as compared to before treatment and/or at the onset of treatment. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days.

In certain embodiments, the methods provided herein result in change (e.g., an increase or a decrease) in the proportion of CD4⁺ CD3⁺ T cells to CD8⁺ CD3⁺ T cells after the subject is treated according to a method provided herein for a set time interval as compared to before treatment and/or at the onset of treatment. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days.

In certain embodiments, the methods provided herein result in an increased virological clearance rate (e.g., increased clearance of SARS-CoV-2 in a subject with COVID-19). In some embodiments, the virological clearance rate is determined based on throat swabs, sputum, and/or lower respiratory tract secretions taken from a treated subject after treatment compared to before treatment after the subject is treated according to a method provided herein for a set time interval as compared to before treatment and/or at the onset of treatment. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days.

In certain embodiments, the methods provided herein result in reduction in level of viral nucleic acid and/or protein (e.g., SARS-CoV-2 nucleic acid and/or protein) present in a subject after treatment compared to before treatment after the subject is treated according to a method provided herein for a set time interval as compared to before treatment and/or at the onset of treatment. In some embodiments, the viral nucleic acid level is determined using RT-PCR. In some embodiments, the viral protein level is determined using an ELISA assay. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days

In certain embodiments, the methods provided herein result in reduction in the time a treated subject spends in an intensive care unit (ICU) compared to untreated subjects. In certain embodiments, the time treated subjects spend in an ICU is reduced by at least 5%, 10%, 15%, 20%, 25%, 30%, 35%, 40%, 45%, 50%, 55%, 60%, 65%, 70%, or 75% compared to untreated subjects.

In certain embodiments, the methods provided herein result in reduction in ventilator requirements of treated subjects compared to untreated subjects. In certain embodiments, the time treated subjects spend on a ventilator is reduced by at least 5%, 10%, 15%, 20%, 25%, 30%, 35%, 40%, 45%, 50%, 55%, 60%, 65%, 70%, or 75% compared to untreated subjects.

In certain embodiments, the methods provided herein result in reduction in mortality of treated subjects compared to untreated subjects. In certain embodiments, the mortality of treated subjects is reduced by at least 5%, 10%, 15%, 20%, 25%, 30%, 35%, 40%, 45%, 50%, 55%, 60%, 65%, 70%, or 75% compared to untreated subjects.

In certain embodiments the methods provided herein result in reduced requirements for oxygen therapy, measured by the ratio of oxygen saturation (SpO2)/fraction of inspired oxygen (FiO2). In certain embodiments, the methods provided herein result in decreased symptom duration, reduced progression along the WHO scale of disease severity, and/or reduced mortality.

### Subjects

In certain aspects, the methods provided herein reduce IL-8, IL-6, IL-1β, and/or TNFα expression levels in a subject in need thereof (e.g., as compared to a standard). In some embodiments, the subject in need thereof suffers from an IL-8, IL-6, IL-1β, and/or TNFα mediated disease or condition. In some embodiments, the subject in need thereof has been infected with a virus (e.g., a respiratory virus). In certain embodiments, the virus is a coronavirus, an influenza virus, and/or a respiratory syncytial virus. In certain embodiments, the virus is a coronavirus such as MERS, SARS (such as SARS-CoV-2). In certain embodiments, the virus is a SARS virus. In certain embodiments, the virus is SARS-CoV-2. In some embodiments, the subject has COVID-19.

In certain aspects, provided herein is a method of treating cytokine storm (cytokine release syndrome) in a subject in need thereof. In some embodiments, the cytokine storm is due to elevation in IL-8, IL-6, IL-1β, and/or TNFα expression levels. In some embodiments, the subject in need thereof has been infected with a virus (e.g., a respiratory virus). In certain embodiments, the virus is a coronavirus, an influenza virus, and/or a respiratory syncytial virus. In certain embodiments, the virus is a coronavirus such as MERS, SARS (such as SARS-CoV-2). In certain embodiments, the virus is a SARS virus. In certain embodiments, the virus is SARS-CoV-2. In some embodiments, the subject has COVID-19.

In some embodiments, the subject in need thereof is present in, is traveling to, and/or has been in a region where viral infection (e.g., coronavirus infection, influenza virus infection, and/or a respiratory syncytial virus infection) is endemic. In certain embodiments, the subject in need thereof is present in, is traveling to, and/or has been in a region where SARS-CoV-2 infection is endemic.

In some embodiments, the subject has been exposed to a source infected with a coronavirus, an influenza virus, and/or a respiratory syncytial virus. In certain embodiments, the subject has been exposed to a source infected with a coronavirus such as MERS, SARS (such as SARS-CoV-2). In certain embodiments, the subject has been exposed to a source infected with SAR-CoV-2.

In certain embodiments, the subject has and/or is at an increased risk for a cardiovascular disease.

In some embodiments, the subject has and/or is at an increased risk for diabetes (e.g., type 2 diabetes).

In certain aspects, provided herein is a method of treating a viral infection in a subject in need thereof, comprising administering to the subject extracellular vesicles from a Prevotella histicola strain comprising at least 99% genomic, 16S and/or CRISPR sequence identity to the nucleotide sequence of the Prevotella histicola Strain B (NRRL accession number B 50329), wherein a Type I interferon response is not reduced, e.g., as determined by IFNα or IFNβ levels.

In certain aspects, provided herein is a method of treating a viral infection in a subject in need thereof, comprising administering to the subject a Prevotella histicola strain comprising at least 99% genomic, 16S and/or CRISPR sequence identity to the nucleotide sequence of the Prevotella histicola Strain B (NRRL accession number B 50329), wherein IFNα and/or IFNβ levels are not reduced.

In certain aspects, provided herein is a method of reducing inflammatory cytokine expression (e.g., reducing IL-8, IL-6, IL-1β, and/or TNFα expression levels) in a subject in need thereof, wherein a Type I interferon response is not reduced, e.g., as determined by IFNα and/or IFNβ levels.

In certain aspects, provided herein is a method of reducing inflammatory cytokine expression (e.g., reducing IL-8, IL-6, IL-1β, and/or TNFα expression levels) in a subject in need thereof, wherein IFNα and/or IFNβ levels are not reduced.

In some embodiments, the subject in need thereof is a child (e.g., a child of no more than 10, 9, 8, 7, 6, 5, 4, 3, 2, or 1 years old). In certain embodiments, the subject is an infant of no more than 12, 11, 10, 9, 8, 7, 6, 5, 4, 3, 2, or 1 months old.

In certain embodiments, the subject is an older adult. In certain embodiments, the subject is at least 50, 55, 60, 65, 70, 75, 80, 80, or 90 years old.

In some embodiments, the subject is a pregnant woman. In some embodiments, the subject is a woman of child-bearing age.

In certain embodiments, the subject is immunocompromised (e.g., a subject who has undergone radiation therapy, immunotherapy, has received a transplant, is taking anti-rejection medication, is taking immunosuppressant medication, is infected with HIV, etc.).

In some embodiments, the subject treated according to the methods provide herein has an IL-8-mediated disease or condition. In certain embodiments, the IL-8 mediated disease or condition comprises Severe Acute Respiratory Syndrome (SARS), influenza, respiratory syncytial viral infection, atherosclerosis, melanoma, ovarian carcinoma, lung cancer, prostate cancer, gastric carcinoma, breast cancer, head-and-neck cancer, colon cancer, colitis-associated cancer, kidney cancer, pancreatic cancer, Crohn's disease (CD), Ulcerative Colitis (UC), Ischemia-Reperfusion injury (IRI), acute lung injury, asthma, chronic obstructive pulmonary disease (COPD), cystic fibrosis (CF), pulmonary fibrosis, multiple sclerosis, psoriasis, atopic dermatitis, rheumatoid arthritis, crescentic glomerulonephritis, IgA nephropathy, membranoproliferative glomerulonephritis, lupus nephritis, or membranous nephropathy, alcoholic hepatitis, or HIV-associated neurocognitive disorder. In certain embodiments, the IL-8 mediated disease or condition comprises a coronavirus, an influenza virus, and/or a respiratory syncytial virus. In certain embodiments, the IL-8 mediated disease or condition comprises a coronavirus such as MERS, SARS (such as SARS-CoV-2). In certain embodiments, the virus is a SARS virus. In certain embodiments, the virus is SARS-CoV-2. In some embodiments, the IL-8 mediated disease is COVID-19.

In some embodiments, the subject treated according to the methods provide herein has an IL-6 mediated disease or condition. In certain embodiments, the IL-6 mediated disease or condition comprises Severe Acute Respiratory Syndrome (SARS), influenza, respiratory syncytial viral infection, Agammaglobulinemia, Amyloidosis, Ankylosing spondylitis, Anti-GBM/Anti-TBM nephritis, Antiphospholipid syndrome, Autoimmune hepatitis, Autoimmune inner ear disease, Atopic dermatitis, Asthma, Castleman disease, Celiac disease, Chagas disease, Chronic recurrent multifocal osteomyelitis, Cogan's syndrome, Cold agglutinin disease, CREST syndrome, Crohn's disease, Dermatomyositis, Devic's disease (neuromyelitis optica), Discoid lupus, Endometriosis, Eosinophilic esophagitis, Eosinophilic fasciitis, Evan's syndrome, Fibromyalgia, Giant cell arteritis, Giant cell myocarditis, Glomerulonephritis, Goodpasture's syndrome, Granulomatosis with polyangiitis, Graves' disease, Guillain-Barre syndrome, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Juvenile arthritis, Juvenile/Type 1 Diabetes, Juvenile myositis, Kawasaki syndrome (Kawasaki Disease (and/or, e.g., Kawasaki disease shock syndrome (KDSS))), Lichen planus, Lichen sclerosis, Lupus (SLE), Meniere's disease, Multiple sclerosis, Myasthenia gravis, Microscopic polyangiitis, Optic neuritis, Pemphigus, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Sjogren's syndrome, Temporal arteritis/Giant cell arteritis, Transverse myelitis, Ulcerative colitis, Uveitis, Vasculitis, Vitiligo, Viral myocarditis, or Wegener's granulomatosis (Granulomatosis with Polyangiitis (GPA)). In certain embodiments, the IL-6 mediated disease or condition comprises a coronavirus, an influenza virus, and/or a respiratory syncytial virus. In certain embodiments, the virus is a SARS virus. In certain embodiments, the IL-6 mediated disease or condition comprises a coronavirus such as MERS, SARS (such as SARS-CoV-2). In certain embodiments, the virus is SARS-CoV-2. In some embodiments, the IL-6 mediated disease mediated disease is COVID-19.

In some embodiments, the subject treated according to the methods provide herein has an IL-10 mediated disease or condition. In certain embodiments, the IL-10 mediated disease or condition comprises Severe Acute Respiratory Syndrome (SARS), influenza, respiratory syncytial viral infection, Agammaglobulinemia, Amyloidosis, Ankylosing spondylitis, Anti-GBM/Anti-TBM nephritis, Antiphospholipid syndrome, Autoimmune hepatitis, Autoimmune inner ear disease, Atopic dermatitis, Asthma, Castleman disease, Celiac disease, Chagas disease, Chronic recurrent multifocal osteomyelitis, Cogan's syndrome, Cold agglutinin disease, CREST syndrome, Crohn's disease, Dermatomyositis, Devic's disease (neuromyelitis optica), Discoid lupus, Endometriosis, Eosinophilic esophagitis, Eosinophilic fasciitis, Evan's syndrome, Fibromyalgia, Giant cell arteritis, Giant cell myocarditis, Glomerulonephritis, Goodpasture's syndrome, Granulomatosis with polyangiitis, Graves' disease, Guillain-Barre syndrome, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Juvenile arthritis, Juvenile/Type 1 Diabetes, Juvenile myositis, Kawasaki syndrome (Kawasaki Disease (and/or, e.g., Kawasaki disease shock syndrome (KDSS))), Lichen planus, Lichen sclerosis, Lupus (SLE), Meniere's disease, Multiple sclerosis, Myasthenia gravis, Microscopic polyangiitis, Optic neuritis, Pemphigus, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Sjogren's syndrome, Temporal arteritis/Giant cell arteritis, Transverse myelitis, Ulcerative colitis, Uveitis, Vasculitis, Vitiligo, Viral myocarditis, or Wegener's granulomatosis (Granulomatosis with Polyangiitis (GPA)). In certain embodiments, the IL-1β mediated disease or condition comprises a coronavirus, an influenza virus, and/or a respiratory syncytial virus. In certain embodiments, the IL-1 mediated disease or condition comprises a coronavirus such as MERS, SARS (such as SARS-CoV-2). In certain embodiments, the virus is a SARS virus. In certain embodiments, the virus is SARS-CoV-2. In some embodiments, the IL-10 mediated disease is COVID-19.

In some embodiments, the subject treated according to the methods provide herein has a TNFα mediated disease or condition. In some embodiments, the TNFα mediated disease or condition is Severe Acute Respiratory Syndrome (SARS), influenza, respiratory syncytial viral infection, rheumatoid arthritis, juvenile chronic arthritis, Crohn's disease (CD), Ulcerative Colitis (UC), ankylosing spondylitis, psoriasis, multiple sclerosis, atherosclerosis, myocardial infarction, heart failure, myocarditis, cardiac allograft rejection, asthma, ischemic renal injury, renal transplant rejection, glomerulonephritis, or inflammatory eye disease. In certain embodiments, the TNFα mediated disease or condition comprises a coronavirus, an influenza virus, and/or a respiratory syncytial virus. In certain embodiments, the TNFα mediated disease or condition comprises a coronavirus such as MERS, SARS (such as SARS-CoV-2). In certain embodiments, the virus is a SARS virus. In certain embodiments, the virus is SARS-CoV-2. In certain embodiments, the virus is SARS-CoV-2. In some embodiments, the TNFα mediated disease is COVID-19.

In some embodiments, the subject treated according to the methods provided herein has autoantibodies, e.g., autoantibodies against type I interferons (e.g., a higher amount of autoantibodies, e.g., than a standard). In some embodiments, the type I interferons are autoantibodies against type I IFN-α2 and/or IFN-β. In some embodiments, the subject has low or undetectable serum IFN-α levels during acute COVID-19. See Bastard et al., Science 370:423 (2020).

In some embodiments, the subject treated according to the methods provided herein has impaired type I interferon (e.g., IFNα and/or IFNβ) production and/or activity (e.g., as compared to a standard). In some embodiments, the subject treated according to the methods provided herein has highly impaired type I interferon (e.g., IFNα and/or IFNβ) production and/or activity (e.g., as compared to a standard). In some embodiments, the subject has no IFNβand low IFNα production and/or activity (e.g., as compared a standard). See Hadjadj et al., Science 369:718-724 (2020).

In some embodiments, the subject treated according to the methods provided herein has a polymorphism in STING (stimulator of interferon (IFN) genes, encoded by TMEM173) that leads to delayed activation and/or over-activation of the STING pathway (e.g., as compared to a standard). See Berthelot and Liote, EBioMedicine 56 (2020).

In some embodiments, the subject treated according to the methods provided herein has diminished and/or delayed IFNλ production (e.g., as compared to a standard). In some embodiments, the subject treated according to the methods provided herein has diminished and/or delayed type I interferon production (e.g., as compared to a cohort control or reference value, e.g., to a standard). See Galani et al., Nature Immunology 22: 32-40 (2021).

In some embodiments, the subject treated according to the methods provided herein has SARS-CoV-2 M protein-mediated impairment (e.g., decreases) in type I and type III interferon production (e.g., as compared to production levels in the absence of COVID-19 infection, e.g., in a standard). In some embodiments, the impairment is due to SARS-CoV-2 M protein targeting of RIG-I/MDA-5 signaling. See Zheng et al., Signal Transduction and Targeted Therapy 5:299 (2020).

In some embodiments, the subject treated according to the methods provided herein has post-acute COVID-19. In some embodiments, the post-acute COVID-19 comprises ongoing symptomatic COVID-19 for people who still have symptoms between 4 and 12 weeks after the start of acute symptoms. In some embodiments, the post-acute COVID-19 comprises post-COVID-19 syndrome wherein subjects have symptoms for more than 12 weeks after the start of acute symptoms. See Venkatesan, The Lancet 9:129 (2021).

In some embodiments, the post-acute COVID-19 comprises gut dysbiosis. See Yeoh et al., Gut 0:1-9 (2021).

As used herein, the standard that is compared to can be a cohort control or reference value or a baseline value (e.g., as compared to a later time point).

### Cytokine Release Syndrome (CRS)

CRS occurs when large numbers of white blood cells, including B cells, T cells, natural killer cells, macrophages, dendritic cells, and monocytes are activated and release inflammatory cytokines, which activate more white blood cells in a positive feedback loop of pathogenic inflammation. See also, Moore et al., Science, 1 May 2020: Vol. 368:6490, pp. 473-474.

CRS or cytokine reactions can occur in a number of infectious diseases including, those associated with infection by COVID-19 (SARS-CoV-2), other coronaviruses, (e.g., SARS-CoV, MERS-CoV), Ebola virus, influenza, cytomegalovirus, variola and group A streptococcus, and sepsis due to infection.

CRS or cytokine reactions can occur in a number of other diseases including multiple sclerosis, pancreatitis, graft-versus-host disease (GVHD), autoimmune disease, acute respiratory distress syndrome (ARDS), multiple organ dysfunction syndromes (including, systemic inflammatory response (SIRS) and secondary hemophagocytic lymphohistiocytosis (sHLH)). CRS has been observed with chimeric antigen receptor (CAR-T) T cell therapy.

See also, Shimabukuro-Vomhagen et al., Journal for ImmunoTherapy of Cancer (2018) 6:56.

In some embodiments, CRS, and/or a condition (such as a viral infection) associated therewith, can be treated with a therapeutic composition and/or a solid dosage form and/or a method provided herein.

Acute lung injury (ALI) can be a common consequence of a cytokine storm in the lung alveolar environment. In some embodiments, ALI can be treated with a therapeutic composition and/or a method provided herein.

### Additional Cellular Factors

As described herein, the therapeutic compositions and methods provided herein can be used to reduce inflammatory cytokine expression (e.g., IL-8, IL-6, IL-1β, and/or TNFα expression) in a subject. For example, the therapeutic compositions and/or a solid dosage forms and/or methods provided herein can be used to treat diseases and conditions associated therewith.

The therapeutic compositions and/or a solid dosage forms and/or methods provided herein can be used to reduce the level of an interleukin, a chemokine, a colony stimulating factor, and/or a tumor necrosis factor (TNF). For example, in addition to IL-8, IL-6, IL-1β, and/or TNFα, the therapeutic compositions and methods provided herein can be used to reduce expression of IL-1Ra, IL-2Ra, IL-7, IL-18, HGF, MCP-1, MCP-3, MIG, M-CSF, GM-CSF, G-CSF, MIG-1α, IP-10, MCP-1, and/or macrophage inflammatory protein (MIP)-1 alpha.

The therapeutic compositions and methods provided herein can be used to change the level of TNF-α, IL-1β, IL-2, IL-6, IL-7, IL-1β, GCSF, IP10, MCP1, MIP1α, sIL-2R, IL-6, and/or IL-8.

### EXAMPLES

### Example 1: Preparation of Lyophilate

The excipient stocks with the formulas provided in Table A to Table D were prepared (amounts shown are percentages of each component in the formula) as solutions. The formulas of the excipient stock fall in to 2 main categories: with and without polymers. The excipient stock solutions were mixed with a liquid preparation of extracellular vesicles. The resulting solutions were freeze-dried and analyzed.

In this Example, the extracellular vesicles (smEVs) used in the studies were isolated from a strain of Prevotella histicola (Prevotella Strain B).

The data collected from the lyophilization of these mixtures is provided in Table E. All measured samples had a residual moisture content of less than 5%. Some samples were additionally tested in vivo using keyhole limpet hemocyanin (KLH)-specific inflammation in a delayed-type hypersensitivity (DTH) model. The samples tested in KLH-DTH showed efficacy.

TABLE A
Excipient stocks for stabilizing extracellular vesicles
during lyophilization. The numerical values given
are on a weight percent basis in the solution.

| | | | | | | Malto- |
|---|---|---|---|---|---|---|
| Formula | Sucrose | Trehalose | Mannitol | Sorbitol | Dextran | dextrin |
| 1 | 40 | 15 | 20 | | 25 | |
| 2 | 20 | 20 | 50 | | 10 | |
| 3 | 50 | | 50 | | | |
| 4 | 40 | 10 | 50 | | | |
| 5 | | 10 | 70 | 0.5 | 19.5 | |
| 7 | | 19.5 | 80 | 0.5 | | |
| 7a | | 20 | 80 | | | |
| 7e | 27 | 20 | 53 | | | |
| 8 | | 10 | 75 | | 15 | |
| 15 | | 19.5 | 70 | 0.5 | 10 | |
| 16 | | 19.5 | 75 | 0.5 | 5 | |
| 17 | | 20 | 80 | | | |
| 18 | | 10 | 60 | | 30 | |
| 19 | | 10 | | | 30 | 60 |
| 20 | | | 100 | | | |

TABLE B
Excipient stocks including polymers for stabilizing extracellular
vesicles during lyophilization. The numerical values given
are on a weight percent basis in the solution.

| Formula | Sucrose | PVP-K30 | Ficoll | Citrate | Arginine |
|---|---|---|---|---|---|
| 6 | 20 | 78 | | 1 | 1 |
| 14 | 20 | | 78 | 1 | 1 |

TABLE C
Excipient stocks including polymers for stabilizing extracellular
vesicles during lyophilization. The numerical values given
are on a weight percent basis in the solution.

| | | | Man- | PVP- | Hydroxypropyl- | |
|---|---|---|---|---|---|---|
| Formula | Sucrose | Trehalose | nitol | K30 | B-cyclodextrin | Ficoll |
| 9 | 20 | 10 | 50 | 20 | | |
| 10 | 10 | 10 | 50 | | 30 | |
| 13 | 20 | 10 | 50 | | | 20 |

TABLE D
Excipient stocks including polymers for stabilizing extracellular
vesicles during lyophilization. The numerical values given
are on a weight percent basis in the solution.

| | | Poloxamer |
|---|---|---|
| Formula | Mannitol | 188 |
| 11 | 95 | 5 |
| 12 | 90 | 10 |

TABLE E
Analytical data obtained for excipient stock solutions used
for the stabilization of extracellular vesicles. “% Stabilizer”
refers to the percentage of the stock solution formula that was added
to a liquid preparation of EVs on a weight basis. “% Moisture”
was determined by Karl Fisher titration. Zₐᵥₑ was determined
by dynamic light scattering (DLS). For particles per mass,
particle numbers were determined by Z-view or NTA instrument;
mass (mg) were decided by analytical balance.

| | | | Zave, | Particles per |
|---|---|---|---|---|
| Formula | % Stabilizer | % Moisture | nm | mass, p/mg |
| — | 0% | | 226.1 | 6.45E+11 |
| 4 | 34% | 2 | 206.2 | 6.28E+10 |
| 5 | 41% | | 209.1 | 6.76E+10 |
| 6 | 35% | 3.6 | 212.8 | 3.25E+10 |
| 7 | 47% | 2.7 | 204 | 7.02E+10 |
| 8 | 44% | 3 | 206.4 | 6.99E+10 |
| 9 | 34% | 2.5 | 187.3 | 7.15E+10 |
| 10 | 34% | 2.7 | 180.1 | 7.37E+10 |
| 11 | 56% | 1.8 | 205.2 | 7.08E+10 |
| 12 | 53% | 1.8 | 202 | 7.66E+10 |
| 13 | 30% | 3 | 172.3 | 7.77E+10 |
| 14 | 35% | 3.8 | 137.4 | 6.12E+10 |
| 15 | 41% | 2.9 | 205.8 | |
| 16 | 44% | 2.8 | 203.9 | |

### Lyophilization Cycle for Extracellular Vesicles (EVs)

The lyophilization cycle is optimized for each excipient formulation. Differences in the critical temperature and collapse temperature of the mixtures mean the shelf temperature during lyophilization is adjusted accordingly. The optimization process involves 3 steps: initial screening, primary drying optimization, and secondary drying optimization. The final cycle is confirmed to be sufficient to dry the material below 5% residual moisture. In this example, the excipient formula chosen for optimization was excipient formula 7.

TABLE F
FORMULA 7 @ 300 MILLITORR

| SHELF TEMP (° C.) | SAMPLE TEMP (° C.) |
|---|---|
| −5 | −17.9 |
| −15 | −23.6 |
| −20 | −26.3 |
| −25 | −28.9 |

TABLE G

| Shelf Temp. | | Primary Drying | Zₐᵥₑ |
|---|---|---|---|
| (° C.) | % Moisture | (hrs) | (nm) |
| −25 | 2.9 | 31.5 | 189 |
| −20 | 2.8 | 28.2 | 215 |
| −15 | 3.3 | 20.8 | 224 |
| −5 | 1.5 | 18.2 | 202 |

TABLE H

| | | Secondary Drying |
|---|---|---|
| Formula #7 | % Moisture | Time (hrs) |
| Primary Drying | 2.8 | 2 |
| Secondary Drying | 2.6 | 29 |

TABLE I
Final lyophilization cycle optimized for extracellular vesicles
stabilized with 47% (by volume) of excipient formula 7.

| | Shelf Temp. | Ramp Time | Hold Time | Vacuum |
|---|---|---|---|---|
| Step | (° C.) | (min) | (min) | (mTorr) |
| Freezing (from RT) | −45 | 200 | 10 | 600K |
| Primary Drying | −20 | 75 | 2,151 | 300 |
| Secondary Drying | 25 | 126 | 300 | 300 |
| Hold | 25 | 0 | 0-300 | 300 |

### Example 2: Orally-Delivered Microbial Extracellular Vesicle Induces Anti-Inflammatory Activity in Mice

Significantly decreased ear swelling and inflammation were observed in a delayed-type hypersensitivity model, showing that extracellular vesicles (EVs) from Prevotella histicola Strain B (NRRL accession number B 50329) modulate systemic inflammatory responses. Activity of the Prevotella histicola EVs is dependent upon both TLR2 signaling and the presence of local immune cells. In vitro results show TLR2 agonism and induction of anti-inflammatory cytokine responses in immune cells by the EVs. These findings demonstrate the anti-inflammatory effects of an orally delivered microbial extracellular vesicle. The Prevotella histicola EVs induce broad-based resolution of inflammation across multiple pathways via a novel mechanism of systemic pharmacology without systemic exposure. EVs are particularly effective at engaging host cells in the gut to modulate distal inflammation. These data point to oral EVs as a new class of immunotherapeutic drugs.

### Example 3: Purification and Preparation of Extracellular Vesicles (EVs) from Bacteria

### Purification

Extracellular vesicles (EVs) are purified and prepared from bacterial cultures using methods known to those skilled in the art (S. Bin Park, et al. PLoS ONE. 6(3):e17629 (2011)).

For example, bacterial cultures are centrifuged at 10,000-15,500×g for 10-40 min at 4° C. or room temperature to pellet bacteria. Culture supernatants are then filtered to include material≤0.22 μm (for example, via a 0.22 μm or 0.45 μm filter) and to exclude intact bacterial cells. Filtered supernatants are concentrated using methods that may include, but are not limited to, ammonium sulfate precipitation, ultracentrifugation, or filtration. Briefly, for ammonium sulfate precipitation, 1.5-3 M ammonium sulfate is added to filtered supernatant slowly, while stirring at 4° C. Precipitations are incubated at 4° C. for 8-48 hours and then centrifuged at 11,000×g for 20-40 min at 4° C. The pellets contain EVs and other debris. Briefly, using ultracentrifugation, filtered supernatants are centrifuged at 100,000-200,000×g for 1-16 hours at 4° C. The pellet of this centrifugation contains EVs and other debris. Briefly, using a filtration technique, using an Amicon Ultra spin filter or by tangential flow filtration, supernatants are filtered so as to retain species of molecular weight>50, 100, 300, or 500 kDa.

Alternatively, EVs are obtained from bacterial cultures continuously during growth, or at selected time points during growth, by connecting a bioreactor to an alternating tangential flow (ATF) system (e.g., XCell ATF from Repligen) according to manufacturer's instructions. The ATF system retains intact cells (>0.22 μm) in the bioreactor, and allows smaller components (e.g., EVs, free proteins) to pass through a filter for collection. For example, the system may be configured so that the <0.22 μm filtrate is then passed through a second filter of 100 kDa, allowing species such as EVs between 0.22 μm and 100 kDa to be collected, and species smaller than 100 kDa to be pumped back into the bioreactor. Alternatively, the system may be configured to allow for medium in the bioreactor to be replenished and/or modified during growth of the culture. EVs collected by this method may be further purified and/or concentrated by ultracentrifugation or filtration as described above for filtered supernatants.

EVs obtained by methods described above may be further purified by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 45% Optiprep in PBS. If filtration was used to concentrate the filtered supernatant, the concentrate is diluted using 60% Optiprep to a final concentration of 45% Optiprep. Samples are applied to a 0-45% discontinuous sucrose gradient and centrifuged at 200,000×g for 3-24 hours at 4° C. Alternatively, high resolution density gradient fractionation could be used to separate EVs based on density.

### Preparation

To confirm sterility and isolation of the EV preparations, EVs are serially diluted onto agar medium used for routine culture of the bacteria being tested and incubated using routine conditions. Non-sterile preparations are passed through a 0.22 μm filter to exclude intact cells. To further increase purity, isolated EVs may be DNase or proteinase K treated.

Alternatively, for preparation of EVs used for in vivo injections, purified EVs are processed as described previously (G. Norheim, et al. PLoS ONE. 10(9): e0134353 (2015)). Briefly, after sucrose gradient centrifugation, bands containing EVs are resuspended to a final concentration of 50 μg/mL in a solution containing 3% sucrose or other solution suitable for in vivo injection known to one skilled in the art. This solution may also contain adjuvant, for example aluminum hydroxide at a concentration of 0-0.5% (w/v).

To make samples compatible with further testing (e.g., to remove sucrose prior to TEM imaging or in vitro assays), samples are buffer exchanged into PBS or 30 mM Tris, pH 8.0 using filtration (e.g., Amicon Ultra columns), dialysis, or ultracentrifugation (following 15-fold or greater dilution in PBS, 200,000×g, 1-3 hours, 4° C.) and resuspension in PBS.

For all of these studies, EVs may be heated, irradiated, and/or lyophilized prior to administration (as described herein).

### Example 4: Manipulating Bacteria Through Stress to Produce Various Amounts of EVs and/or to Vary Content of EVs

Stress, and in particular envelope stress, has been shown to increase production of EVs by some bacterial strains (I. MacDonald, M. Kuehn. J Bacteriol 195(13): doi: 10/1128/JB.02267-12). In order to vary production of EVs by bacteria, bacteria are stressed using various methods.

Bacteria may be subjected to single stressors or stressors in combination. The effects of different stressors on different bacteria is determined empirically by varying the stress condition and determining the IC50 value (the conditions required to inhibit cell growth by 50%). EV purification, quantification, and characterization occurs. EV production is quantified (1) in complex samples of bacteria and EVs by nanoparticle tracking analysis (NTA) or transmission electron microscopy (TEM); or (2) following EV purification by NTA, lipid quantification, or protein quantification. EV content is assessed following purification by methods described above.

### Antibiotic Stress

Bacteria are cultivated under standard growth conditions with the addition of sublethal concentrations of antibiotics. This may include 0.1-1 μg/mL chloramphenicol, or 0.1-0.3 μg/mL gentamicin, or similar concentrations of other antibiotics (e.g., ampicillin, polymyxin B). Host antimicrobial products such as lysozyme, defensins, and Reg proteins may be used in place of antibiotics. Bacterially-produced antimicrobial peptides, including bacteriocins and microcins may also be used.

Temperature Stress

Bacteria are cultivated under standard growth conditions, but at higher or lower temperatures than are typical for their growth. Alternatively, bacteria are grown under standard conditions, and then subjected to cold shock or heat shock by incubation for a short period of time at low or high temperatures respectively. For example, bacteria grown at 37° C. are incubated for 1 hour at 4° C.-18° C. for cold shock or 42° C.-50° C. for heat shock.

Starvation and Nutrient Limitation

To induce nutritional stress, bacteria are cultivated under conditions where one or more nutrients are limited. Bacteria may be subjected to nutritional stress throughout growth or shifted from a rich medium to a poor medium. Some examples of media components that are limited are carbon, nitrogen, iron, and sulfur. An example medium is M9 minimal medium (Sigma-Aldrich), which contains low glucose as the sole carbon source. Particularly for Prevotella spp., iron availability is varied by altering the concentration of hemin in media and/or by varying the type of porphyrin or other iron carrier present in the media, as cells grown in low hemin conditions were found to produce greater numbers of EVs (S. Stubbs et al. Letters in Applied Microbiology. 29:31-36 (1999). Media components are also manipulated by the addition of chelators such as EDTA and deferoxamine.

Saturation

Bacteria are grown to saturation and incubated past the saturation point for various periods of time. Alternatively, conditioned media is used to mimic saturating environments during exponential growth. Conditioned media is prepared by removing intact cells from saturated cultures by centrifugation and filtration, and conditioned media may be further treated to concentrate or remove specific components.

Salt Stress

Bacteria are cultivated in or exposed for brief periods to medium containing NaCl, bile salts, or other salts.

UV Stress

UV stress is achieved by cultivating bacteria under a UV lamp or by exposing bacteria to UV using an instrument such as a Stratalinker (Agilent). UV may be administered throughout the entire cultivation period, in short bursts, or for a single defined period following growth.

Reactive Oxygen Stress

Bacteria are cultivated in the presence of sublethal concentrations of hydrogen peroxide (250-1,000 μM) to induce stress in the form of reactive oxygen species. Anaerobic bacteria are cultivated in or exposed to concentrations of oxygen that are toxic to them.

Detergent Stress

Bacteria are cultivated in or exposed to detergent, such as sodium dodecyl sulfate (SDS) or deoxycholate.

pH Stress

Bacteria are cultivated in or exposed for limited times to media of different pH.

### Example 5: Profiling EV Composition and Content

EVs may be characterized by any one of various methods including, but not limited to, NanoSight characterization, SDS-PAGE gel electrophoresis, Western blot, ELISA, liquid chromatography-mass spectrometry and mass spectrometry, dynamic light scattering, lipid levels, total protein, lipid to protein ratios, nucleic acid analysis and/or zeta potential.

### NanoSight Characterization of EVs

Nanoparticle tracking analysis (NTA) is used to characterize the size distribution of purified EVs. Purified EV preparations are run on a NanoSight machine (Malvern Instruments) to assess EV size and concentration.

### SDS-PAGE Gel Electrophoresis

To identify the protein components of purified EVs, samples are run on a gel, for example a Bolt Bis-Tris Plus 4-12% gel (Thermo-Fisher Scientific), using standard techniques. Samples are boiled in 1× SDS sample buffer for 10 minutes, cooled to 4° C., and then centrifuged at 16,000×g for 1 min. Samples are then run on a SDS-PAGE gel and stained using one of several standard techniques (e.g., Silver staining, Coomassie Blue, Gel Code Blue) for visualization of bands.

### Western Blot Analysis

To identify and quantify specific protein components of purified EVs, EV proteins are separated by SDS-PAGE as described above and subjected to Western blot analysis (Cvjetkovic et al., Sci. Rep. 6, 36338 (2016)) and are quantified via ELISA.

### EV Proteomics and Liquid Chromatography-Mass Spectrometry (LC-MS/MS) and Mass Spectrometry (MS)

Proteins present in EVs are identified and quantified by Mass Spectrometry techniques. EV proteins may be prepared for LC-MS/MS using standard techniques including protein reduction using dithiotreitol solution (DTT) and protein digestion using enzymes such as LysC and trypsin as described in Erickson et al, 2017 (Molecular Cell, VOLUME 65, ISSUE 2, P361-370, Jan. 19, 2017). Alternatively, peptides are prepared as described by Liu et al. 2010 (JOURNAL OF BACTERIOLOGY, June 2010, p. 2852-2860 Vol. 192, No. 11), Kieselbach and Oscarsson 2017 (Data Brief. 2017 February; 10: 426-431), Vildhede et al, 2018 (Drug Metabolism and Disposition Feb. 8, 2018). Following digestion, peptide preparations are run directly on liquid chromatography and mass spectrometry devices for protein identification within a single sample. For relative quantitation of proteins between samples, peptide digests from different samples are labeled with isobaric tags using the iTRAQ Reagent-8plex Multiplex Kit (Applied Biosystems, Foster City, CA) or TMT 10plex and 11plex Label Reagents (Thermo Fischer Scientific, San Jose, CA, USA). Each peptide digest is labeled with a different isobaric tag and then the labeled digests are combined into one sample mixture. The combined peptide mixture is analyzed by LC-MS/MS for both identification and quantification. A database search is performed using the LC-MS/MS data to identify the labeled peptides and the corresponding proteins. In the case of isobaric labeling, the fragmentation of the attached tag generates a low molecular mass reporter ion that is used to obtain a relative quantitation of the peptides and proteins present in each EV.

Additionally, metabolic content is ascertained using liquid chromatography techniques combined with mass spectrometry. A variety of techniques exist to determine metabolomic content of various samples and are known to one skilled in the art involving solvent extraction, chromatographic separation and a variety of ionization techniques coupled to mass determination (Roberts et al 2012 Targeted Metabolomics. Curr Protoc Mol Biol. 30: 1-24; Dettmer et al 2007, Mass spectrometry-based metabolomics. Mass Spectrom Rev. 26(1):51-78). As a non-limiting example, a LC-MS system includes a 4000 QTRAP triple quadrupole mass spectrometer (AB SCIEX) combined with 1100 Series pump (Agilent) and an HTS PAL autosampler (Leap Technologies). Media samples or other complex metabolic mixtures (˜10 μL) are extracted using nine volumes of 74.9:24.9:0.2 (v/v/v) acetonitrile/methanol/formic acid containing stable isotope-labeled internal standards (valine-d8, Isotec; and phenylalanine-d8, Cambridge Isotope Laboratories). Standards may be adjusted or modified depending on the metabolites of interest. The samples are centrifuged (10 minutes, 9,000 g, 4° C.), and the supernatants (10 μL) are submitted to LCMS by injecting the solution onto the HILIC column (150×2.1 mm, 3 μm particle size). The column is eluted by flowing a 5% mobile phase [10 mM ammonium formate, 0.10% formic acid in water] for 1 minute at a rate of 250 uL/minute followed by a linear gradient over 10 minutes to a solution of 40% mobile phase [acetonitrile with 0.1% formic acid]. The ion spray voltage is set to 4.5 kV and the source temperature is 450° C.

The data are analyzed using commercially available software like Multiquant 1.2 from AB SCIEX for mass spectrum peak integration. Peaks of interest should be manually curated and compared to standards to confirm the identity of the peak. Quantitation with appropriate standards is performed to determine the number of metabolites present in the initial media, after bacterial conditioning. A non-targeted metabolomics approach may also be used using metabolite databases, such as but not limited to the NIST database, for peak identification.

### Dynamic Light Scattering (DLS)

DLS measurements, including the distribution of particles of different sizes in different EV preparations are taken using instruments such as the DynaPro NanoStar (Wyatt Technology) and the Zetasizer Nano ZS (Malvern Instruments).

### Lipid Levels

Lipid levels are quantified using FM4-64 (Life Technologies), by methods similar to those described by A. J. McBroom et al. J Bacteriol 188:5385-5392. and A. Frias, et al. Microb Ecol. 59:476-486 (2010). Samples are incubated with FM4-64 (3.3 μg/mL in PBS for 10 minutes at 37° C. in the dark). After excitation at 515 nm, emission at 635 nm is measured using a Spectramax M5 plate reader (Molecular Devices). Absolute concentrations are determined by comparison of unknown samples to standards (such as palmitoyloleoylphosphatidylglycerol (POPG) vesicles) of known concentrations. Lipidomics can be used to identify the lipids present in the EVs.

### Total Protein

Protein levels are quantified by standard assays such as the Bradford and BCA assays. The Bradford assays are run using Quick Start Bradford 1× Dye Reagent (Bio-Rad), according to manufacturer's protocols. BCA assays are run using the Pierce BCA Protein Assay Kit (Thermo-Fisher Scientific). Absolute concentrations are determined by comparison to a standard curve generated from BSA of known concentrations. Alternatively, protein concentration can be calculated using the Beer-Lambert equation using the sample absorbance at 280 nm (A280) as measured on a Nanodrop spectrophotometer (Thermo-Fisher Scientific). In addition, proteomics may be used to identify proteins in the sample.

### Lipid:Protein Ratios

Lipid:protein ratios are generated by dividing lipid concentrations by protein concentrations. These provide a measure of the purity of vesicles as compared to free protein in each preparation.

### Nucleic Acid Analysis

Nucleic acids are extracted from EVs and quantified using a Qubit fluorometer. Size distribution is assessed using a BioAnalyzer and the material is sequenced.

### Zeta Potential

The zeta potential of different preparations are measured using instruments such as the Zetasizer ZS (Malvern Instruments).

### Example 6: Manufacturing Conditions

Enriched media is used to grow and prepare the bacteria for in vitro and in vivo use and, ultimately, for EV preparations. For example, media may contain sugar, yeast extracts, plant-based peptones, buffers, salts, trace elements, surfactants, anti-foaming agents, and vitamins. Composition of complex components such as yeast extracts and peptones may be undefined or partially defined (including approximate concentrations of amino acids, sugars etc.). Microbial metabolism may be dependent on the availability of resources such as carbon and nitrogen. Various sugars or other carbon sources may be tested. Alternatively, media may be prepared and the selected bacterium grown as shown by Saarela et al., J. Applied Microbiology. 2005. 99: 1330-1339, which is hereby incorporated by reference. Influence of fermentation time, cryoprotectant and neutralization of cell concentrate on freeze-drying survival, storage stability, and acid and bile exposure of the selected bacterium produced without milk-based ingredients.

At large scale, the media is sterilized. Sterilization may be accomplished by Ultra High Temperature (UHT) processing. The UHT processing is performed at very high temperature for short periods of time. The UHT range may be from 135-180° C. For example, the medium may be sterilized from between 10 to 30 seconds at 135° C.

Inoculum can be prepared in flasks or in smaller bioreactors and growth is monitored. For example, the inoculum size may be between approximately 0.5 and 3% of the total bioreactor volume. Depending on the application and need for material, bioreactor volume can be at least 2 L, 10 L, 80 L, 100 L, 250 L, 1000 L, 2500 L, 5000 L, 10,000 L.

Before the inoculation, the bioreactor is prepared with medium at desired pH, temperature, and oxygen concentration. The initial pH of the culture medium may be different that the process set-point. pH stress may be detrimental at low cell centration; the initial pH could be between pH 7.5 and the process set-point. For example, pH may be set between 4.5 and 8.0. During the fermentation, the pH can be controlled through the use of sodium hydroxide, potassium hydroxide, or ammonium hydroxide. The temperature may be controlled from 25° C. to 45° C., for example at 37° C. Anaerobic conditions are created by reducing the level of oxygen in the culture broth from around 8 mg/L to 0 mg/L. For example, nitrogen or gas mixtures (N2, CO2, and H2) may be used in order to establish anaerobic conditions. Alternatively, no gases are used and anaerobic conditions are established by cells consuming remaining oxygen from the medium. Depending on strain and inoculum size, the bioreactor fermentation time can vary. For example, fermentation time can vary from approximately 5 hours to 48 hours.

Reviving bacteria from a frozen state may require special considerations. Production medium may stress cells after a thaw; a specific thaw medium may be required to consistently start a seed train from thawed material. The kinetics of transfer or passage of seed material to fresh medium, for the purposes of increasing the seed volume or maintaining the microbial growth state, may be influenced by the current state of the bacteria (ex. exponential growth, stationary growth, unstressed, stressed).

Inoculation of the production fermenter(s) can impact growth kinetics and cellular activity. The initial state of the bioreactor system must be optimized to facilitate successful and consistent production. The fraction of seed culture to total medium (e.g., a percentage) has a dramatic impact on growth kinetics. The range may be 1-5% of the fermenter's working volume. The initial pH of the culture medium may be different from the process set-point. pH stress may be detrimental at low cell concentration; the initial pH may be between pH 7.5 and the process set-point. Agitation and gas flow into the system during inoculation may be different from the process set-points. Physical and chemical stresses due to both conditions may be detrimental at low cell concentration.

Process conditions and control settings may influence the kinetics of microbial growth and cellular activity. Shifts in process conditions may change membrane composition, production of metabolites, growth rate, cellular stress, etc. Optimal temperature range for growth may vary with strain. The range may be 20-40° C. Optimal pH for cell growth and performance of downstream activity may vary with strain. The range may be pH 5-8. Gasses dissolved in the medium may be used by cells for metabolism. Adjusting concentrations of 02, CO2, and N2 throughout the process may be required. Availability of nutrients may shift cellular growth. Bacteria may have alternate kinetics when excess nutrients are available.

The state of bacteria at the end of a fermentation and during harvesting may impact cell survival and activity. Bacteria may be preconditioned shortly before harvest to better prepare them for the physical and chemical stresses involved in separation and downstream processing. A change in temperature (often reducing to 20-5° C.) may reduce cellular metabolism, slowing growth (and/or death) and physiological change when removed from the fermenter. Effectiveness of centrifugal concentration may be influenced by culture pH. Raising pH by 1-2 points can improve effectiveness of concentration but can also be detrimental to cells. Bacteria may be stressed shortly before harvest by increasing the concentration of salts and/or sugars in the medium. Cells stressed in this way may better survive freezing and lyophilization during downstream.

Separation methods and technology may impact how efficiently bacteria are separated from the culture medium. Solids may be removed using centrifugation techniques. Effectiveness of centrifugal concentration can be influenced by culture pH or by the use of flocculating agents. Raising pH by 1-2 points may improve effectiveness of concentration but can also be detrimental to cells. Bacteria may be stressed shortly before harvest by increasing the concentration of salts and/or sugars in the medium. Cells stressed in this way may better survive freezing and lyophilization during downstream. Additionally, Bacteria may also be separated via filtration. Filtration is superior to centrifugation techniques for purification if the cells require excessive g-minutes to successfully centrifuge. Excipients can be added before after separation. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Harvesting can be performed by continuous centrifugation. Product may be resuspended with various excipients to a desired final concentration. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Lyophilization of material, including live bacteria, vesicles, or other bacterial derivative includes a freezing, primary drying, and secondary drying phase. Lyophilization begins with freezing. The product material may or may not be mixed with a lyoprotectant or stabilizer prior to the freezing stage. A product may be frozen prior to the loading of the lyophilizer, or under controlled conditions on the shelf of the lyophilizer. During the next phase, the primary drying phase, ice is removed via sublimation. Here, a vacuum is generated and an appropriate amount of heat is supplied to the material. The ice will sublime while keeping the product temperature below freezing, and below the material's critical temperature (Tc). The temperature of the shelf on which the material is loaded and the chamber vacuum can be manipulated to achieve the desired product temperature. During the secondary drying phase, product-bound water molecules are removed. Here, the temperature is generally raised higher than in the primary drying phase to break any physico-chemical interactions that have formed between the water molecules and the product material. After the freeze-drying process is complete, the chamber may be filled with an inert gas, such as nitrogen. The product may be sealed within the freeze dryer under dry conditions, in a glass vial or other similar container, preventing exposure to atmospheric water and contaminates.

### Example 7: smEV Preparation

Prevotella histicola smEVs were prepared as follows.

smEVs: Downstream processing of smEVs began immediately following harvest of the bioreactor. Centrifugation at 20,000 g was used to remove the cells from the broth. The resulting supernatant was clarified using 0.22 m filter. The EVs were concentrated and washed using tangential flow filtration (TFF) with flat sheet cassettes ultrafiltration (UF) membranes with 100 kDa molecular weight cutoff (MWCO). Diafiltration (DF) was used to washout small molecules and small proteins using 5 volumes of phosphate buffer solution (PBS). The retentate from TFF was spun down in an ultracentrifuge at 200,000 g for 1 hour to form a pellet rich in EVs called a high-speed pellet (HSP). The pellet was resuspended with minimal PBS and a gradient was prepared with Optiprep™ density gradient medium and ultracentrifuged at 200,000 g for 16 hours. Of the resulting fractions, 2 middle bands contained EVs. The fractions were washed with 15 fold PBS and the EVs spun down at 200,000 g for 1 hr to create the fractionated HSP or fHSP. It was subsequently resuspended with minimal PBS, pooled, and analyzed for particles per mL and protein content. Dosing was prepared from the particle/mL count to achieve desired concentration. The EVs were characterized using a NanoSight NS300 by Malvern Panalytical in scatter mode using the 532 nm laser.

### Example 8: EV Isolation and Enumeration

The equipment used in EV isolation includes a Sorvall RC-5C centrifuge with SLA-3000 rotor; an Optima XE-90 Ultracentrifuge by Beckman-Coulter 45Ti rotor; a Sorvall wX+ Ultra Series Centrifuge by Thermo Scientific; and a Fiberlite F37 L-8x100 rotor.

### Bacterial Supernatant Collection and Filtration

Bacteria must be pelleted and filtered away from supernatant in order to recover EVs and not bacteria.

Pellet bacterial culture is generated by using a Sorvall RC-5C centrifuge with the SLA-3000 rotor and centrifuge culture for a minimum of 15 min at a minimum of 7,000 rpm. And then decanting the supernatant into new and sterile container.

The supernatant is filtered through a 0.2 μm filter. For supernatants with poor filterability (less than 300 ml of supernatant pass through filter) a 0.45 μm capsule filter is attached ahead of the 0.2 μm vacuum filter. The filtered supernatant is stored at 4° C. The filtered supernatant can then be concentrated using TFF.

Isolation of EVs using Ultracentrifugation

Concentrated supernatant is centrifuged in the ultracentrifuge to pellet EVs and isolate the EVs from smaller biomolecules. The speed is for 200,000 g, time for 1 hour, and temperature at 4° C. When rotor has stopped, tubes are removed from the ultracentrifuge and the supernatant is gently poured off More supernatant is added the tubes are centrifuged again. After all concentrated supernatant has been centrifuged, the pellets generated are referred to as ‘crude’ EV pellets. Sterile 1×PBS is added to pellets, which are placed in a container. The container is placed on a shaker set at speed 70, in a 4° C. fridge overnight or longer. The EV pellets are resuspended with additional sterile 1×PBS. The resuspended crude EV samples are stored at 4° C. or at −80° C.

EV Purification using Density Gradients

Density gradients are used for EV purification. During ultracentrifugation, particles in the sample will move, and separate, within the graded density medium based on their ‘buoyant’ densities. In this way EVs are separated from other particles, such as sugars, lipids, or other proteins, in the sample.

For EV purification, four different percentages of the density medium (60% Optiprep) are used, a 45% layer, a 35% layer, a 25%, and a 15% layer. This will create the graded layers. A 0% layer is added at the top consisting of sterile 1×PBS. The 45% gradient layer should contain the crude EV sample. 5 ml of sample is added to 15 ml of Optiprep. If crude EV sample is less than 5 ml, bring up to volume using sterile 1×PBS.

Using a serological pipette, the 45% gradient mixture is pipetted up and down to mix. The sample is then pipetted into a labeled clean and sterile ultracentrifuge tube. Next, a 10 ml serological pipette is used to slowly add 13 ml of 35% gradient mixture. Next 13 ml of the 25% gradient mixture is added, followed by 13 ml of the 15% mixture and finally 6 ml of sterile 1×PBS. The ultracentrifuge tubes are balanced with sterile 1×PBS. The gradients are carefully placed in a rotor and the ultracentrifuge is set for 200,000 g and 4° C. The gradients are centrifuged for a minimum of 16 hours.

A clean pipette is used to remove fraction(s) of interest, which are added to 15 ml conical tube. These ‘purified’ EV samples are kept at 4° C.

In order to clean and remove residual optiprep from EVs, 10× volume of PBS are added to purified EVs. The ultracentrifuge is set for 200,000 g and 4° C. Centrifuge and spun for 1 hour. The tubes are carefully removed from ultracentrifuge and the supernatant decanted. The purified EVs are washed until all sample has been pelleted. 1×PBS is added to the purified pellets, which are placed in a container. The container is placed on a shaker set at speed 70 in a 4° C. fridge overnight or longer. The ‘purified’ EV pellets are resuspended with additional sterile 1×PBS. The resuspended purified EV samples are stored at 4° C. or at −80° C.

### Example 9: Additional Stocks for Drying

EVs from Prevotella histicola bacteria are dried, such as by freeze drying or spray drying using one of the stocks provided in Table K.

TABLE K
Stocks comprising excipients by relative concentration (% w:w).

| Formula | Full composition |
|---|---|
| 1 | 40% Sucrose, 15% Trehalose, 20% Mannitol, 25% Dextran 40k |
| 2 | 20% Sucrose, 20% Trehalose, 50% Mannitol, 10% Dextran 40k |
| 3 | 50% Sucrose, 50% Mannitol |
| 4 | 40% Sucrose, 10% Trehalose, 50% Mannitol |
| 5 | 10% Trehalose, 70% Mannitol, 0.5% Sorbitol, |
| | 19.5% Dextran 40k |
| 6 | 20% Sucrose, 78% PVP-K30, 1% Citrate, 1% Arginine |
| 7 | 19.5% Trehalose, 80% Mannitol, 0.5% Sorbitol |
| 7a | 20% Trehalose, 80% Mannitol |
| 7e | 27% Sucrose, 20% Trehalose, 53% Mannitol |
| 8 | 10% Trehalose, 75% Mannitol, 15% Dextran 40k |
| 9 | 20% Sucrose, 10% Trehalose, 50% Mannitol, 20% PVP-K30 |
| 10 | 10% Sucrose, 10% Trehalose, 50% Mannitol, |
| | 30% B-Cyclodextrin |
| 11 | 95% Mannitol, 5% Poloxamer 188 |
| 12 | 90% Mannitol, 10% Poloxamer 188 |
| 13 | 20% Sucrose, 10% Trehalose, 50% Mannitol, 20% Ficoll |
| 14 | 20% Sucrose, 78% Ficoll, 1% Citrate, 1% Arginine |
| 15 | 19.5% Trehalose, 70% Mannitol, 0.5% Sorbitol, |
| | 10% Dextran 40k |
| 16 | 19.5% Trehalose, 75% Mannitol, 0.5% Sorbitol, |
| | 5% Dextran 40k |
| 17 | 20% Trehalose, 80% Mannitol |
| 18 | 10% Trehalose, 60% Mannitol, 30% Dextran 40k |
| 19 | 10% Trehalose, 30% Dextran 40k, 60% Maltodextrin |
| 20 | 100% Mannitol |
| 21 | 20% Mannitol, 60% PEG 6000, 20% Trehalose |
| 22 | 10% Mannitol, 60% PEG 6000, 30% Trehalose |
| 23 | 70% PEG 6000, 30% Trehalose |
| 24 | 70% PEG 6000, 30% Mannitol |

The drying conditions in Table L are used for lyophilization.

TABLE L
General conservative lyophilization cycle for EVs.

| | Ramp | Hold time | Shelf temp | Vacuum |
|---|---|---|---|---|
| Step | time (min) | (min) | (C.) | (mTorr) |
| Freezing | 200 | 360 | −45 | 100-300 |
| Primary drying | 75 | 5000 | −20 | 100-300 |
| Secondary | 180 | 1000 | 25 | 100-300 |
| drying | | | | |
| Hold | 0 | N/A | 25 | 100-300 |

### Example 10: Spray-Dried Powders of Prevotella histicola smEVs

In this Example, the extracellular vesicles (smEVs) used in the studies were isolated from Prevotella histicola Strain B.

The smEVs were spray dried as follows:

EV retentate was mixed with one of the excipients provided in Table P.

TABLE P
Stocks comprising excipients by relative concentration (% w:w)

| Formula | Full composition |
|---|---|
| 7a | 80% Mannitol, 20% Trehalose |
| 25 | 100% Trehalose |
| 26 | Maltodextrin-Trehalose (20:60:20) |
| 27 | Maltodextrin-Trehalose (70:30) |
| 28 | PEG6000-Trehalose (70:30) |
| 29 | Mannitol-Maltodextrin-Trehalose (20:60:20) |

The spray drying was performed at 100° C. or 130° C. Temperatures are also included in Table Q.

Post spray drying, each powder was analyzed for moisture content (MC) (by Karl Fischer titration (KF)) and particles (particles/mg spray-dried powder (p/mg)) (quantification by nanoparticle tracking analysis (NTA) using Zetaview). Results are shown in Table Q. EXP7A is stock of formula 7a.

TABLE Q
Moisture content and particle content of spray dried samples

| | Inlet | Moisture | |
|---|---|---|---|
| | temperature | Content | Particles/ |
| Sample ID | (° C.) | (%) | mg |
| EXP7A-130° C. inlet | 130 | 3.64 | 1.30E+10 |
| EXP7A-100° C. inlet | 100 | 2.54 | 1.25E+10 |
| Man-Malt-Tre | 130 | 5.35 | 1.40E+10 |
| (20:60:20) | | | |
| Malt-Tre (70:30) | 130 | 8.38 | 2.00E+10 |
| 100% Trehalose | 130 | 6.37 | 1.60E+10 |

Man: mannitol; Malt: maltodextrin; Tre: trehalose.

Spray drying was also performed using a stock that consisted of PEG6000-Mannitol-Trehalose (60:20:20). However, less dried product was recovered relative to other methods and was not analyzed further.

Prevotella histicola smEVs were spray dried or lyophilized in stock of formulation 7a (F7A) at two concentrations: 25× and 500×, with an inlet temperature of 130° C.

The comparison of particles/mg spray-dried powder and size are shown in Table R. SD=spray dried; L0.47=lyophilized; 0.47 refers to the stock ratio used: 47 g of excipient with every 100 g of retentate.

The particle packing and size of the spray dried and lyophilized EVs were similar with both methods of drying.

TABLE R

| Sample | Particles/mg | CV | Size (nm) |
|---|---|---|---|
| 25X L0.47 F7A | 3.35e+9 | 2.10% | 172.7 |
| 25X SD F7A | 3.95e+9 | 0.00% | 164.8 |
| 500X L0.47 F7A | 5.50e+10 | 0.00% | 165.1 |
| 500X SD F7A | 4.88e+10 | 0.70% | 163.8 |

### Example 11: Prevotella histicola smEVs and Anti-TNFα Antibody: DTH Efficacy

In this Example, the extracellular vesicles (smEVs) used in the studies were isolated from Prevotella histicola Strain B.

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were injected intraperitoneally on day 0, 3 and 6 with 3 mg/kg of anti-TNF alpha (Anti-TNFα) antibody (Clone: XT3.11 purchased from BioXCell) or equivalent isotype control (IgG1 also purchased from BioXCell). Mice were orally gavaged daily with Prevotella histicola extracellular vesicles (smEVs) or dosed intraperitoneally with dexamethasone at 1 mg/kg from days 5-8. After dosing on day 8, mice were anaesthetized with isoflurane, left ears were measured for baseline measurements with Fowler calipers and the mice were challenged intradermally with KLH in saline (10 μl) in the left ear. Ear thickness measurements were taken at 24 hours.

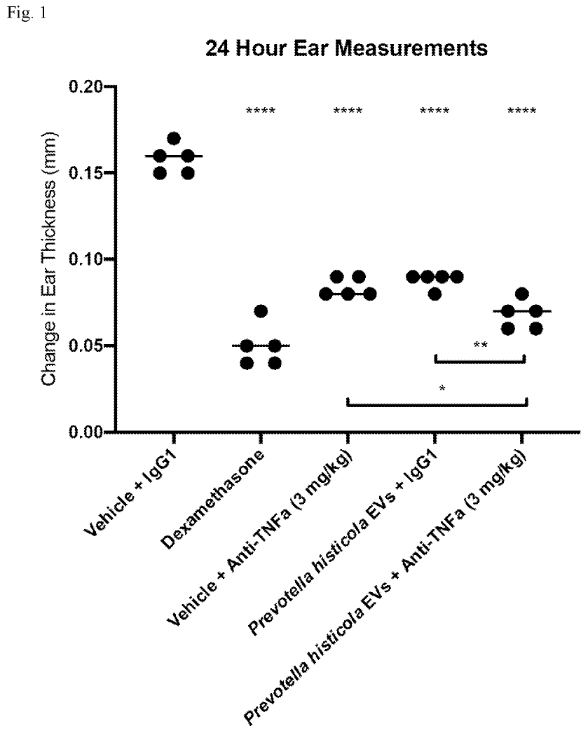

The 24 hour ear measurement results are shown in FIG. 1. smEVs made from Prevotella histicola (at 2 E09 particles per dose) were tested alone or in combination with 3 mg/kg anti-TNF alpha antibody or equivalent isotype control (IgG1). Three mg/kg anti-TNF alpha antibody were also tested alone or with vehicle control. The combination of Prevotella histicola smEVs with anti-TNF alpha was more efficacious than both Prevotella histicola smEVs alone and anti-TNF alpha antibody alone.

### Example 12: Preparation of a Solid Dosage Form Comprising Prevotella histicola smEVs

Tablets of the recipes in Table S were prepared:

TABLE S
Compositions of active tablets (400 mg)

| | Low | Medium | High |
|---|---|---|---|
| Tablets | Strength | Strength | Strength |
| Prevotella histicola smEVs | 10% | 10% | 62.5% |
| (drug substance/powder) | (LS DS) | (HS DS) | (HS DS) |
| SMCC HD90 | 72.5% | 72.5% | 20.0% |
| Crospovidone | 15.0% | 15.0% | 15.0% |
| SiO₂ | 1.0% | 1.0% | 1.0% |
| Magnesium stearate | 1.5% | 1.5% | 1.5% |
| Tablet weight (mg) | 400 | 400 | 400 |

The Prevotella histicola smEVs in Table S are from strain Prevotella histicola Strain B 50329 (NRRL accession number B 50329).

Drug substance (powder) was prepared by lyophilization using excipient formula 7a. HS DS: high strength drug substance. LS DS: low strength drug substance. LS DS was prepared by diluting HS DS 10× (using lyophilization excipients) before lyophilization.

To prepare the pharmaceutical composition tablets, wet granulation was performed on the drug substance (pharmaceutical agent) containing the smEVs. Drug substance was (i) mixed with water; (ii) dried on a fluid bed dryer; (iii) milled; (iv) then blended with the drug product excipients provided in Table S.

The tablets were 5.5 mm×15.8 mm.

### Example 13: Preparation of a Solid Dosage Form Comprising Prevotella histicola smEVs

Capsule of the recipes in Table T were prepared:

TABLE T
Compositions of active capsules

| | Low | Medium | High |
|---|---|---|---|
| Capsules | Strength | Strength | Strength |
| Prevotella histicola smEVs | 10% | 10% | 90% |
| (drug substance/powder) | (LS DS) | (HS DS) | (HS DS) |
| Mannitol | 87.5% | 87.5% | 7.5% |
| SiO2 | 1.0% | 1.0% | 1.0% |
| Magnesium stearate | 1.5% | 1.5% | 1.5% |
| Capsule filled weight (mg) | 360 | 360 | 360 |

The Prevotella histicola smEVs in Table T are from strain Prevotella histicola Strain B 50329 (NRRL accession number B 50329).

Drug substance (powder) was prepared by lyophilization using excipient formula 7a. HS DS: high strength drug substance. LS DS: low strength drug substance. LS DS was prepared by diluting HS DS 10× (using lyophilization excipients) before lyophilization. HS DS: high strength drug substance. LS DS: low strength drug substance.

LS DS was prepared by diluting HS DS 10× (using lyophilization excipients) before lyophilization.

To prepare the pharmaceutical composition capsules, wet granulation was performed on the drug substance (pharmaceutical agent) containing the smEVs. Drug substance was (i) mixed with water; (ii) dried on a fluid bed dryer; (iii) milled; (iv) then blended with the drug product excipients provided in Table T.

The capsules were size 0.

### Example 14: COVID-19 Risk Assessment

Antiviral responses are activated rapidly after viral infection in order to control and prevent dissemination of the virus. Virus infection results in two general types of immune response. The first is a rapid-onset innate immune response against the virus, which involves the synthesis of Type 1 interferons and the stimulation of Natural Killer (NK) cells. If the infection proceeds beyond the first few rounds of viral replication, the innate immune response will trigger the adaptive immune response. The adaptive immune response itself has two components, the humoral response (the synthesis of virus-specific antibodies by B lymphocytes) and the cell-mediated response (the synthesis of specific CD8+ cytotoxic T lymphocytes that kill infected cells). Both of these components of the adaptive immune response result also in the production of long-lived memory cells that allow for a much more rapid response to a subsequent infection with the same virus. Thus, an immune competent host should be able to mount both an innate and adaptive immune response. Much of the coverage of COVID immunity often focuses on antibody response which can neutralize viruses like SARS-CoV-2. But as variants like Omicron evolve to evade antibodies, the role of another part of the immune system, T cells, has been brought into sharper focus. These immune cells work in a different way to antibodies, attacking infected cells rather than the virus itself, which can make their response broader and more robust. Now, research is showing that, unlike antibodies, T cell potency is not impacted by the mutations in variants like Omicron. T-cell levels do not tend to fade as quickly as antibodies after an infection or vaccination. And because T cells can recognize many more sites along the spike protein than can antibodies, they are better able to recognize mutated variants. Thus, having an intact T cell response is likely important for both mounting a successful anti-viral response as well as maintaining immune memory of viruses previously encountered.

Prevotella histicola EVs are obtained from Prevotella Strain B. Prevotella histicola EVs are formulated as a lyophilized powder. Preclinically, Prevotella histicola EVs are administered orally and are not systemically bioavailable. Prevotella histicola EVs exert their anti-inflammatory effects on peripheral tissue through engagement of cells of the intestine, including intestinal epithelial cells and immune cells in the lamina propria.

Prevotella histicola EVs have been shown in preclinical mouse inflammation models to reduce antigen-specific T cell responses, without impacting:
The production of anti-viral TLR3-mediated Type I interferons (alpha and beta) by spleen cells of mice undergoing an inflammatory response
Interferon-gamma production by murine T cells
The generation of functional CD4+Th1 cells in a mouse model of inflammation
Production of interferon-gamma from human memory CD8 T cells in response to a viral peptide pool (Cytomegalovirus, Epstein-Bar virus, and Influenza virus)

Taken together, the data demonstrate that treatment with Prevotella histicola EVs does not result in general immuno-suppression of multiple immune pathways but is effective through a selective restoration of immune homeostasis.

In this Example, the extracellular vesicles (smEVs) used in the studies were isolated from Prevotella histicola Strain B.

Prevotella histicola smEVs Resolve Tissue Inflammation in a KLH Delayed-Type Hypersensitivity (DTH) Model

Mice were immunized by subcutaneous injection with Keyhole Limpet Hemocyanin (KLH) emulsified with Complete Freund's Adjuvant. On Day 1 after the sensitization, mice were dosed for 15 days with vehicle (phosphate buffered saline) and Prevotella histicola smEVs by oral gavage, or dexamethasone by intraperitoneal injection. On day 15, mice were challenged by intradermal ear injection with KLH. The DTH response was evaluated 24 hours post-challenge.

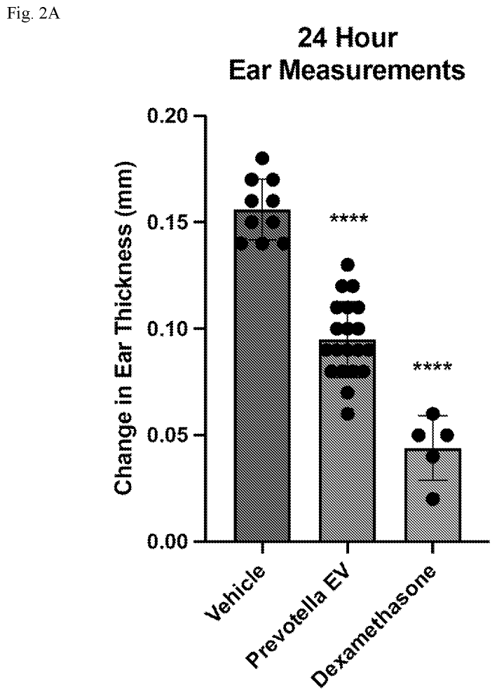

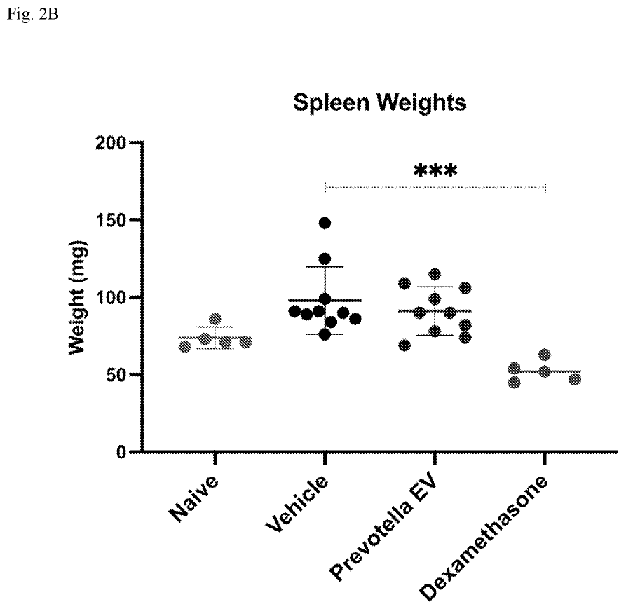

Results: 15 days of dosing with Prevotella histicola EVs or dexamethasone significantly inhibited ear inflammation (FIG. 2A). Spleen weights at the end of the study showed that although systemic treatment with dexamethasone significantly reduced spleen size, as expected from a systemic immuno-suppressive drug, Prevotella histicola EVs did not change the spleen weight (FIG. 2B). These data suggest that Prevotella histicola EVs can inhibit tissue inflammation without broader immuno-suppressive effects.

### Production of Type 1 Interferons by Spleen Lymphocytes Ex Vivo

Interferons (IFNs) are cytokines that are secreted by host cells in response to virus infection and are the body's first line of antiviral defense. By inducing the expression of hundreds of IFN-stimulated genes, several of which have antiviral functions, IFNs block virus replication at many levels. There are two main types of IFN, type I and type II. Type I or ‘viral’ IFNs include IFN-α, IFN-β, IFN-ω and IFN-τ; type II IFN is IFN-γ. Most cell types can produce IFN-α and IFN-β, which are the best-characterized type I IFNs, whereas IFN-γ is produced only by certain cells of the immune system, including natural killer (NK) cells, CD4+T helper 1 (TH1) cells and CD8+ cytotoxic T cells. Drugs with broad immuno-suppressant activity, such as corticosteroids, can inhibit the production of IFNs, which can lead to delayed viral clearance and adverse outcomes in various viral pneumonias. Therefore, a therapeutic approach that ameliorates inflammation without blocking the anti-viral IFN immune response would have an optimal safety profile.

In order to study the effect of Prevotella histicola EV treatment on Type I and II IFN production, at the end of a DTH study, spleen cells were isolated and then restimulated in vitro with PMA and ionomycin or polyinosinic-polycytidylic acid (poly I:C). PMA activates protein kinase C, while ionomycin is a calcium ionophore, and stimulation with these compounds bypasses the T cell membrane receptor complex and will lead to activation of several intracellular signaling pathways, resulting in T cell activation and production of a variety of cytokines. Poly I:C is a molecule that mimics viral double-strained RNA, and a potent ligand for Toll-like receptor 3, which induces interferon-alpha and interferon-beta from immune cells.

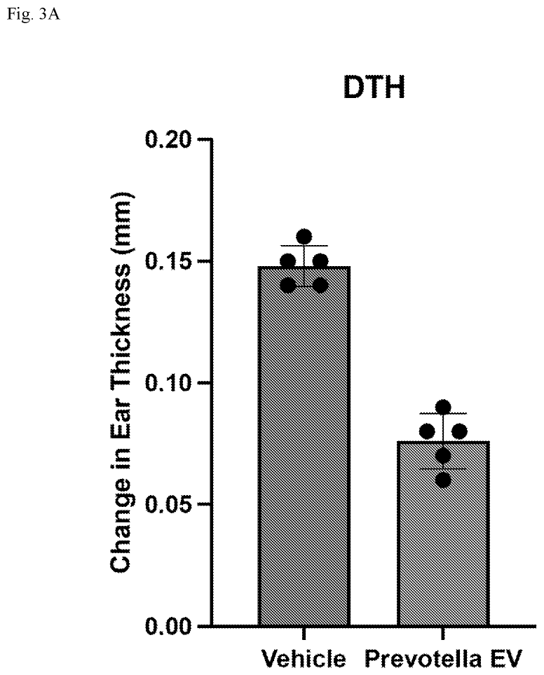

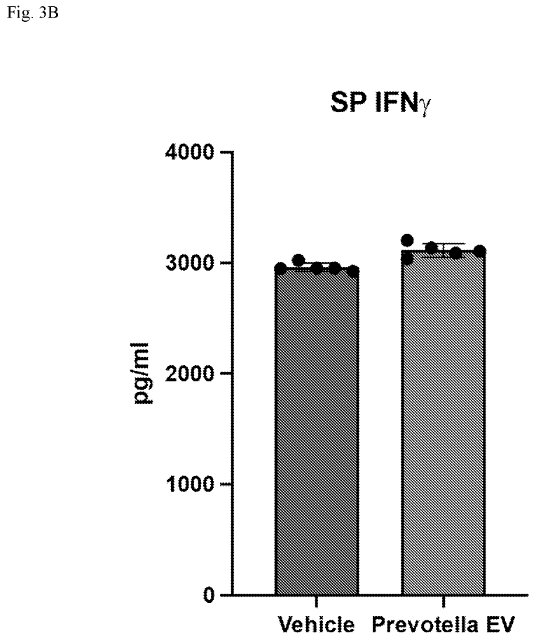

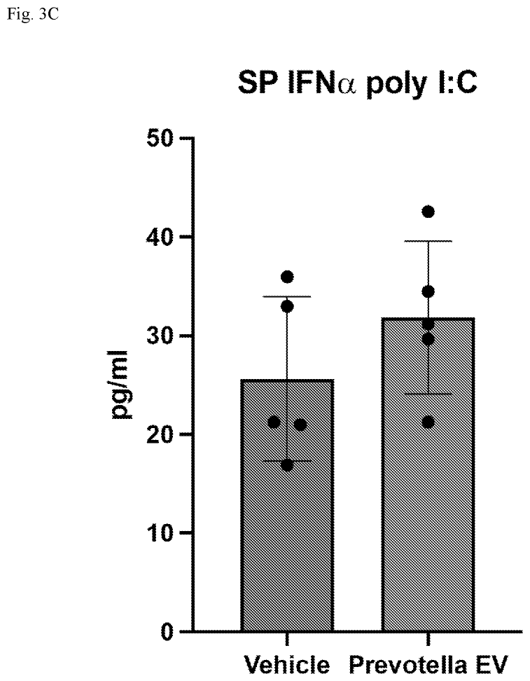

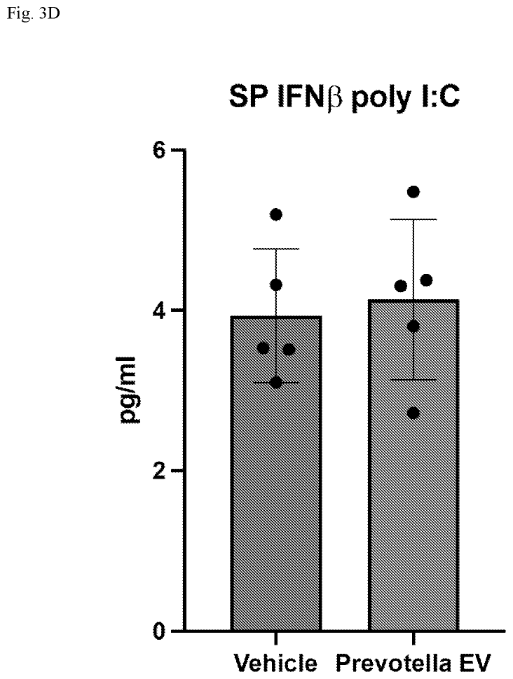

Results: Four days of dosing with Prevotella histicola EVs significantly inhibited ear inflammation in the DTH model (FIG. 3A). Stimulation of the spleen cells from these mice led to the production of IFN-γ (FIG. 3B), IFN-α (FIG. 3C), IFN-β (FIG. 3D). Treatment with Prevotella histicola EVs had no impact on the production of these IFNs compared to the vehicle control. This demonstrates that Prevotella histicola EVs selectively inhibit tissue inflammation while preserving protective Type I and II interferon responses.

Mouse CD4 TH1 T Cell Responses are not Reduced by Prevotella histicola EVs Treatment in DTH

CD4 T cells help B cells to produce antibodies and help CD8+ T cells to kill virus-infected cells. One of the dominant cytokines produced by T cells is interferon gamma, a key player in controlling viral infection. One report has described a higher proportion of IFN-γ-producing T helper 1 (TH1)-like cells in patients with moderate disease than in patients with severe disease. Moreover, CD4+ T cells specific for the SARS-CoV-2 spike protein have been identified in acute infection and have a TH1 cell cytokine profile. In order to determine whether treatment with Prevotella histicola EVs reduces the numbers of TH1 T cells or inhibits their production of cytokines such as IFN-γ and TNF, CD4 T cells were isolated from spleens and lymph nodes after a DTH response, and TH1 T cell numbers were determined by flow cytometry.

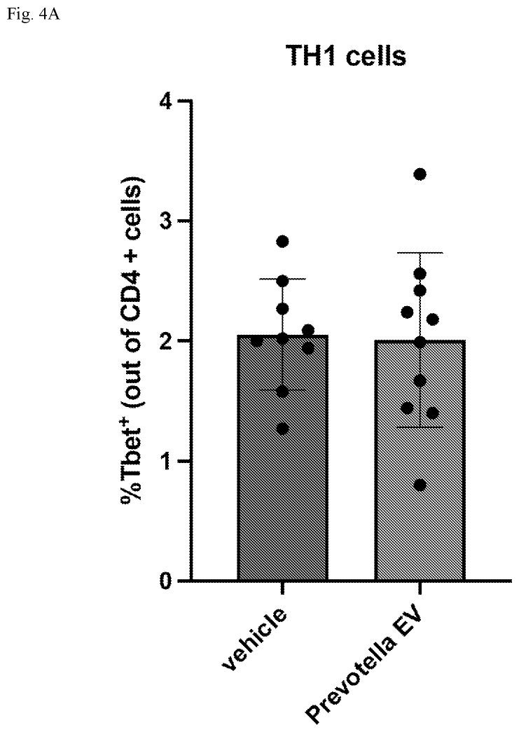

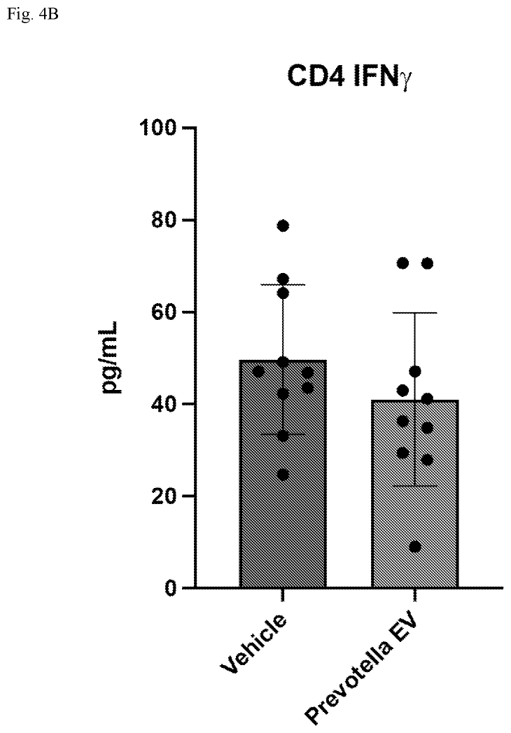

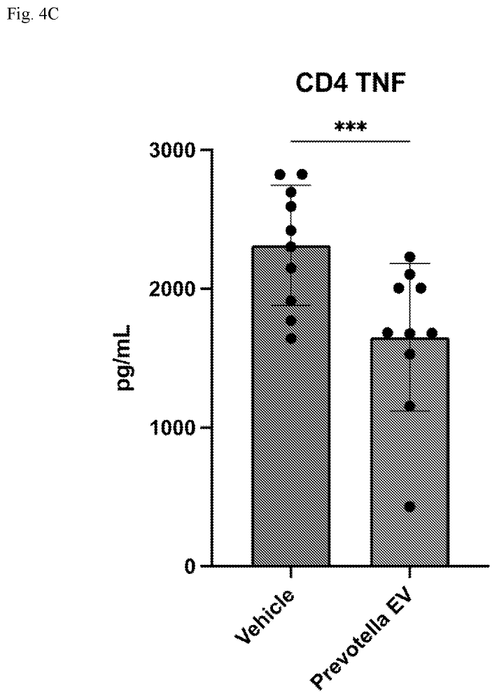

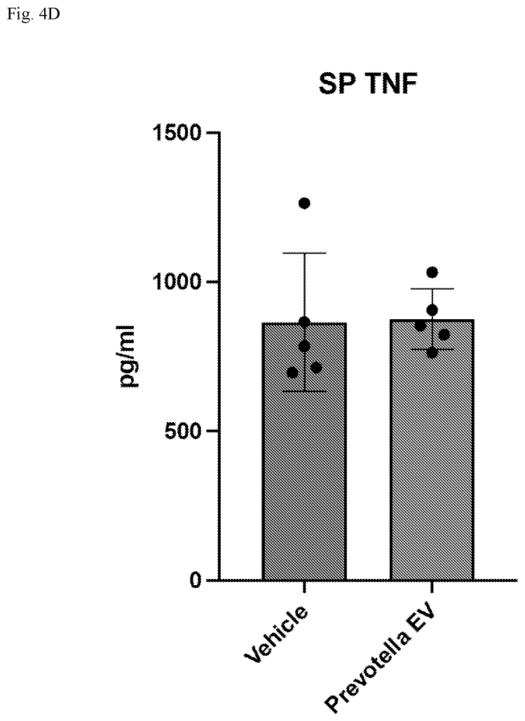

Results: TH1 T cells, which are defined by their expression of the transcription factor T-bet, were of a similar proportion (roughly 2%) of total CD4 T cells in mice treated with either vehicle or Prevotella histicola EVs (FIG. 4A). Although PMA/ionomycin-stimulated CD4 T cells from mice treated with either vehicle or Prevotella histicola EVs produced the same amount of IFN-γ (FIG. 4B), those from mice treated with Prevotella histicola EVs did produce about 25% less TNF than those from vehicle-treated mice (FIG. 4C). However, the levels of TNF produced by PMA/ionomycin-treated spleen cells from vehicle- or Prevotella histicola EV-treated mice were similar (FIG. 4D), indicating that the overall production of TNF is not impaired by Prevotella histicola EV treatment.

Human Dendritic Cells (DCs) Exposed to Prevotella histicola EVs Enhance the Production of Viral Defense Cytokines in a Mixed DC:CD8 T Cell Co-Culture

In addition to CD4+ T cells and neutralizing antibodies, CD8+ T cells contribute to protective immune responses against SARS-CoV-2 in patients with COVID-19. In adaptive immunity, CD8+ T cells play an essential role in controlling viral infection by killing virus-infected cells and producing effector cytokines such as IFN-γ. DCs that present viral antigens to CD8 T cells also produce cytokines such as IL-12, TNF, and IL-6, which then induce the differentiation and activation of IFN-γ-producing CD8 T cells.

An in vitro assay with primary human dendritic cells (DCs) and autologous CD8+ T cells was carried out to measure the capacity of Prevotella histicola EVs to modulate both DCs and antigen-specific CD8+ T cell responses. Primary human DCs from the blood of 3 healthy human donors were differentiated in vitro for 7 days. To assess the immuno-modulatory properties of Prevotella histicola EVs, DCs were incubated with 3 doses of Prevotella histicola EVs for 24 hours. After 24 hours of EV conditioning, EVs were removed from the DC culture and autologous human CD8+ T cells and CEF Class I peptide pool was added. The CEF peptide pool is composed of peptides from Cytomegalovirus, Epstein Bar virus, and Influenza virus, pathogens to which the majority of the human population has been exposed. Controls used were DCs only, DCs+ T Cells only, and DCs+ T cells+CEF peptide. After 24 hours of stimulation with CEF peptide, DC-CD8+ T cell supernatants were collected, and cytokines produced by both DCs and CD8 T cells were measured.

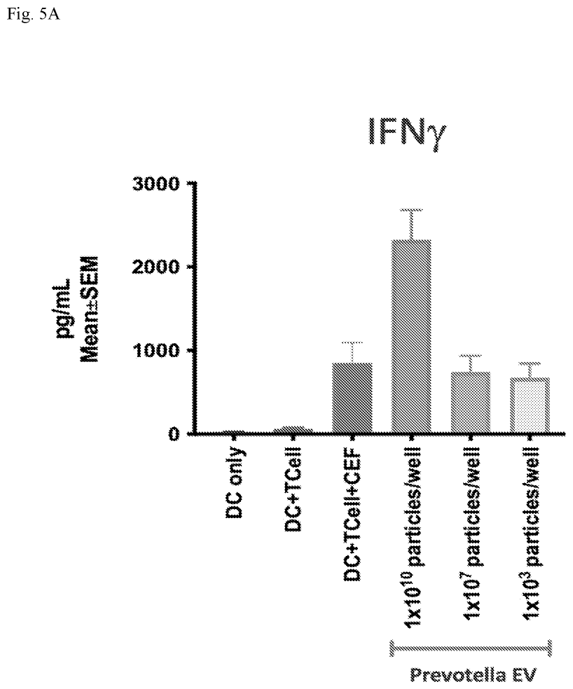

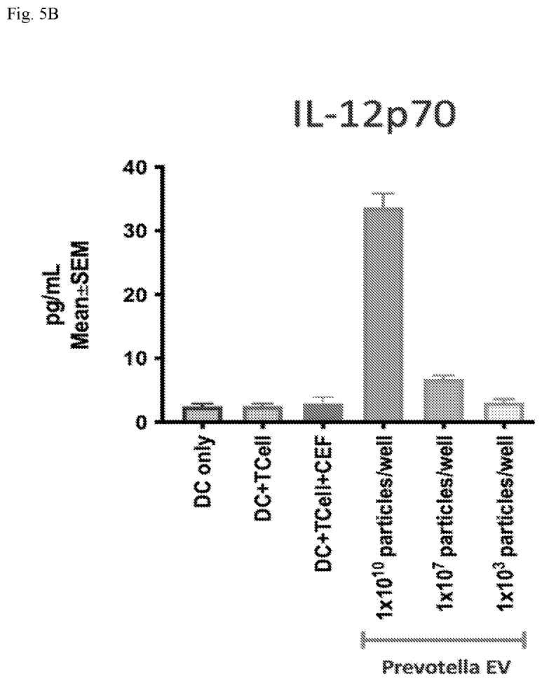

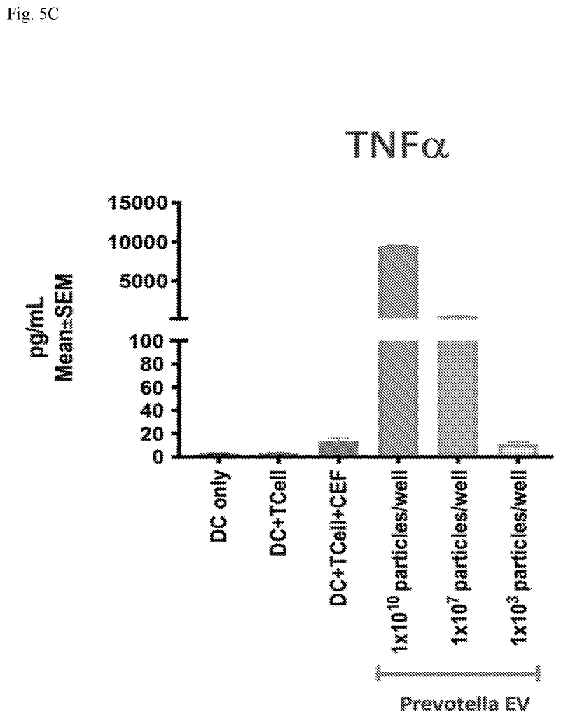

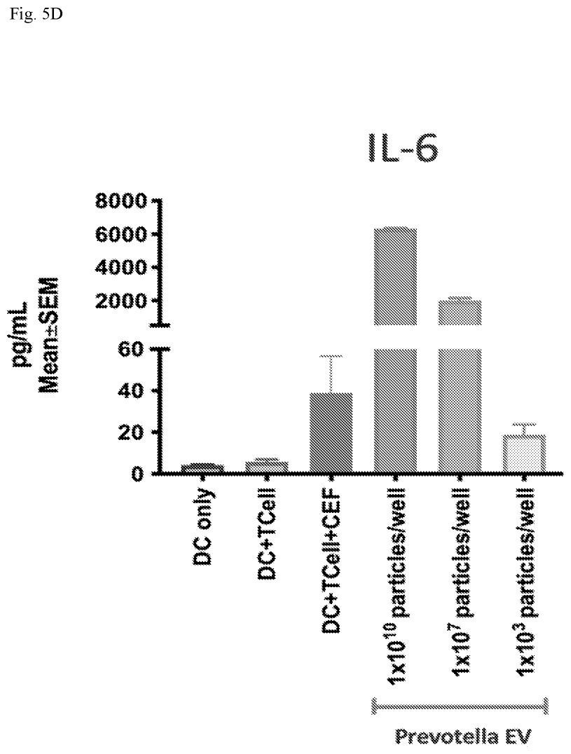

Results: When human DCs were incubated with Prevotella histicola EVs, the IFNγ response to CEF was increased at the highest dose (1×10¹⁰ particles/well), but not changed at the two lower doses compared to the DC+CD8 T cell+CEF peptide co-culture control (FIG. 5A). Similarly, other cytokines produced primarily by DCs (IL-12p70, TNFα, and IL-6) were increased at the highest dose (IL-12p70) or at the two higher doses of Prevotella histicola EVs, compared to the DC+CD8 T cell+CEF peptide control (FIGS. 5B-5D). These data indicate that exposure of dendritic cells to Prevotella histicola EVs does not impair the ability of antigen-specific CD8 T cells to produce the anti-viral cytokine IFN-γ, and indeed, at higher levels IFN-γ may be enhanced. Similarly, DCs themselves can respond to Prevotella histicola EVs by producing higher levels of inflammatory cytokines which in vivo have been shown to be critical for protective host responses to viral infection.

### Conclusion

The combination of in vitro, in vivo, and ex vivo data demonstrate that Prevotella histicola EVs do not broadly impair either innate or adaptive immune responses. Prevotella histicola EVs are orally delivered and gut restricted, and its systemic effects are exerted through local interactions with cells of the gastro-intestinal tract. Anti-viral responses such as CD4 and CD8 T cell production of IFN-γ, innate anti-viral production of IFN-α and IFN-β, and the generation of effector T cell populations are all preserved after treatment with Prevotella histicola EVs.

The data demonstrate that treatment with Prevotella histicola EVs results in resolution of peripheral inflammation without leading to immunosuppression of the host anti-viral response.

### Example 15: A Study Investigating the Safety and Efficacy of Prevotella Histicola Strain B smEVs in the Treatment of Patients Hospitalized with SARS-CoV-2 Infection

### Background

The COVID-19 pandemic, as declared on Mar. 11, 2020 by World Health Organization (WHO), is caused by a novel coronavirus (SARS-Cov-2). It is estimated to result in ˜50,000-160,000 deaths in the USA, if optimal healthcare can be delivered, and up to in excess of 2.2 million deaths if healthcare resources such as ventilated beds are exhausted (Cookson 2020). It is the pulmonary complications of the viral infection that results in the majority of hospitalizations, admissions to ICU and ultimately death (Guan 2020; Huang 2020; Liu 2020; Wang 2020). The COVID-Related Complications (CRC) include acute respiratory distress syndrome (ARDS), arrhythmia, shock, acute kidney injury, acute cardiac injury, liver dysfunction and secondary infection (Huang 2020; Maharaj 2020). Significant symptoms that do not result in hospitalization are also common and result in significant illness even short of hospitalization.

Study of coronavirus infections in tissue culture and animal models and of historical, SARS-coronavirus outbreaks, provide insights into the likely pathophysiology of infection with COVID-19 (Guan 2020; Gralinski 2015). The majority of tissue damage following infection with SARS-Cov1 appears to be due to a later, exaggerated, host immune response (Gralinski 2015). The host anti-viral response is driven by the induction of type I interferons which inhibit transcription and translation of the viral genome and reduce the threshold for activation of natural killer cells. Type I interferons also decrease expression of Serping 1, a regulator of the complement system and coagulation proteases; this may lead to complement-mediated tissue damage and a prothrombotic tendency. In airway epithelial cells, type I IFNs upregulate expression of ACE2 in airway epithelial cells. Whereas ACE2 has been shown to be protective in models of acute lung injury, it is also the receptor for the spike protein of COVID-19 and is used by the virus for binding to its target cells.

While SARS-CoV-2 infection evades detection by the immune system in the first 24 h of infection, after 7-14 days following symptom onset an exaggerated response from the host immune system occurs in a subgroup of people. This leads to progressive lung damage leading to the need for hospitalization and oxygen therapy that can progress to severe pulmonary complications requiring ventilation and even death. It is important to note that the development of Diffuse Alveolar Damage (DAD) is often independent of high-titer viral replication (Peiris 2003). Other end organ damage can also occur secondary to the host immune response. This abnormal immune and inflammatory response in affected lungs includes production of high levels of IL-6, IL-8, TNFα, IL-1β, influx of neutrophils and cytotoxic T cells. A Th₂ (IL4, IL13) response from alternatively-activated macrophages, and an associated profibrotic phenotype (including increased TGF β and PDGFα production) can lead to lung fibrosis and chronic sequelae (Ruan 2020). Activation of the coagulation cascade is associated with development of fibrin clots in the alveoli. IL-6 and IL-8 are increased in subjects hospitalized with coronaviral infections (Mehta 2020). A therapeutic agent with anti-inflammatory effects across IL-6, IL-8 and TNFα could prevent this host immune mediated organ damage. The host immune response is clearly important in the initial anti-viral response of the host. A prolonged and exaggerated immune response as measured by these cytokines/chemokines is however associated with pulmonary complications, hospitalization and ultimately death. A therapeutic agent that does not abrogate the initial host anti-viral immune response but modulates the delayed excess immune response via multiple pathways, restoring a state of immune homeostasis, could offer significant clinical benefit to subjects with COVID-19 infections.

Rationale: An exaggerated host immune response leads to the life-threatening complications of COVID-19 infection. The cytokine IL-6 and chemokine IL-8 have been shown to be increased in subjects hospitalized with coronaviral infections, infections with influenza A, and in secondary HLH, and their exaggerated levels are pathogenic in the development of complications such as ARDS. The host immune response is clearly important in the initial anti-viral response of the host and IL-6 in particular has been shown to be important in the early phase of the infection. A prolonged and exaggerated immune response is however associated with pulmonary complications, hospitalization and ultimately death. A therapeutic agent that does not abrogate the host immune response entirely, but instead modulates multiple pathways and returns it back to a state of immune homeostasis, could offer significant clinical benefit to subjects with coronaviral infections.

The profile of Prevotella histicola Strain B extracellular vesicles (smEVs) as an oral agent with the ability to modulate multiple key immune pathways without blocking them completely—that is, immune normalization rather than immune suppression—could offer significant clinical benefit to patients at risk of developing serious complications secondary to COVID-19.

TABLE 1
Clinical Trial Objectives and Endpoints

| Objectives | Endpoints |
|---|---|
| Primary | |
| To evaluate the effect of Prevotella | Change from baseline to the lowest S/F ratio |
| histicola Strain B smEVs on pulmonary | measured in days 1-14 |
| function as measured by the change in | |
| Oxygen Saturation (SpO2)/Fraction of | |
| Inspired Oxygen (FiO2) [S/F] ratio | |
| Secondary | |
| To evaluate the effect of Prevotella | Change in S/F ratio at days 4, 7, 10 and |
| histicola Strain B smEVs on the | 14/discharge day. |
| development and severity of | Percentage change in S/F ratio at days 4, 7, |
| complications of COVID-19 infection | 10 and 14/discharge day. |
| | Percentage of participants at each level on |
| | the WHO OSCI score at days 4, 7, 14, 21 |
| | and 42. |
| | Percentage of participants with shifts from |
| | each level of the WHO OSCI score at |
| | baseline at days 4, 7, 14, 21 and 42. |
| | Percentage of participants remaining at their |
| | baseline score on the WHO OSCI (or lower) |
| | at days 4, 7, 14, 21 and 42. |
| | Percentage of participants reporting each |
| | level of the WHO OSCI score at their worst |
| | post-baseline day. |
| | The time in days spent at each participant's |
| | worst reported WHO OSCI score |
| | (excluding death). |
| | Intubation and mechanical-ventilation free |
| | survival, defined as the time in days from |
| | start of treatment to first occurrence of a |
| | WHO OSCI score of 6 or more. |
| | Overall survival, defined as the time in days |
| | from start of treatment to death by any |
| | cause |
| | Number of days requiring oxygen therapy. |
| | Number of days with pyrexia ≥ 38° C. |
| | Maximum daily temperature. |
| | Minimum SpO2 level. |
| | Maximum SpO2 level. |
| To evaluate the effect of Prevotella | Time to discharge, defined as the time in |
| histicola Strain B smEVs on length of | days from start of treatment to first |
| hospitalization and recovery in | occurrence of a WHO OSCI score of 2 or |
| participants with COVID-19 | less. |
| | Time to oxygen saturation (SpO2) ≥94% on |
| | room air without further requirement for |
| | oxygen therapy. |
| | Time to recovery, defined as the time in |
| | days from symptom onset to alleviation of |
| | all COVID-19 symptoms. |
| To evaluate the safety and tolerability of | Incidence and severity of treatment |
| Prevotella histicola Strain B smEVs in | emergent adverse events and serious |
| participants with COVID-19 | adverse events. |
| | Incidence of clinically significant abnormal |
| | changes in safety laboratory parameters. |
| Exploratory | |
| To evaluate the effect of Prevotella | Change from baseline in cytokine levels |
| histicola Strain B smEVs on the | (including IL-6) at day 4 and day 7 (and/or |
| exaggerated host cytokine response to | at additional timepoints if samples are |
| COVID-19 infection | available). |
| | Change from baseline in inflammatory |
| | response at day 4 and day 7 (and/or at |
| | additional timepoints if samples are |
| | available). |
| To identify clinical or biochemical | Statistical significance of each potential |
| predictors of response to Prevotella | predictor-treatment interaction in |
| histicola Strain B smEVs, and to confirm | exploratory models based on the final |
| lack of systemic absorption. | selected model for the primary efficacy |
| | analysis. |
| | To identify the presence of Prevotella |
| | histicola Strain B smEVs in stool and/or |
| | blood using PCR primers |

### Overall Design:

This is a randomized, placebo-controlled clinical study to assess the safety and efficacy of Prevotella histicola Strain B smEVs in patients hospitalized with COVID-19 infection. The study is designed to evaluate the efficacy of Prevotella histicola Strain B smEVs at reducing time to resolution of symptoms, preventing progression of COVID-19 symptoms and preventing COVID-Related Complications (CRC). This is a pilot study with a primary objective of investigating the potential of Prevotella histicola Strain B smEVs in the prevention of COVID-19 disease progression. The secondary objective is to evaluate multiple endpoints for clinical relevance and sensitivity, while informing the sample size for future studies. Where possible, data will be taken from assessments performed as part of the participant's routine clinical care in this pragmatic study.

Participants who are hospitalized with confirmed COVID-19 disease and are confirmed to be eligible for the study will be randomized to either the active (Prevotella histicola Strain B smEVs) or placebo group (1:1 randomization), in addition to standard of care. Dosing will be initiated on a twice daily regime for the first 3 days (6 doses) and then once daily for the remaining 11 days (14 days total treatment course). The trial hypothesis is that treatment with Prevotella histicola Strain B smEVs in hospitalized patients reduces oxygen requirements by normalizing the exaggerated host immune response to COVID-19. This will be measured by assessing the ratio of the Oxygen Saturation (SpO2)/Fraction of Inspired Oxygen (FiO2), which is a validated measure of severity of ARDS.

Dosing will be stopped if participants are admitted to ICU, efficacy and safety data will continue to be collected according to the schedule of activities, where practical. However, if the participant is eligible for another interventional trial at this point, they may be enrolled into it, and withdrawn from this study, after discussion with the chief investigator. Inclusion in concurrent interventional studies will not be permitted. Inclusion in observational studies in parallel to this study is allowed.

### Participant Trial Duration:

This study will consist of a 14-day treatment period followed by a 28-day post-treatment follow-up visit.

An initial twice daily (bd) dosing regimen has been selected to maximize the speed of response. Prevotella histicola Strain B smEVs work via direct interaction with immune cells in the epithelium of the upper small intestine. A twice a day regimen doubles the duration of exposure of the smEVs to the immune cells in the upper small intestine per 24 hours.

### Study Population:

This protocol contains participants with a confirmed diagnosis of COVID-19 viral infection.

### Drug Product:

Treatment will be with Prevotella histicola Strain B smEVs in a solid dosage form, such as a capsule or tablet.

Efficacy Assessments:

Oxygen Saturation: Oxygen saturation will be measured using a peripheral pulse oximeter and will also be analyzed as a ratio with the oxygen flow (SpO2/FiO2). The measurement will ideally be performed with the subject sitting and having been rested for at least 10 minutes.

If the subject is on 3 litres/min oxygen flow or less, and the investigator feels it is safe to do so, the investigator will remove the subject's supplemental oxygen for 10 minutes while they remain seated, and while continuously monitoring the oxygen saturation. After 10 minutes the oxygen saturation reading will be taken to calculate the S/F ratio on room air. If, during this process, the saturations drop by greater than 4%, the oxygen will be immediately replaced and the ratio measured on oxygen.

WHO Ordinal Scale: The WHO ordinal scale (Table 2) will be collected throughout the study. This is an accepted instrument which has been developed specifically for trials in patients with COVID-19.

TABLE 2
WHO ordinal scale for clinical improvement (OSCI) of COVID-19

| Patient State | Descriptor | Score |
|---|---|---|
| Uninfected | No clinical or virological | 0 |
| | evidence of infection | |
| Ambulatory | No limitation of activities | 1 |
| | Limitation of activities | 2 |
| Hospitalized | Hospitalized, no oxygen | 3 |
| Mild disease | therapy | |
| | Oxygen by mask or nasal | 4 |
| | prongs | |
| Hospitalized | Non-invasive ventilation or | 5 |
| Severe Disease | high-flow oxygen | |
| | Intubation and mechanical | 6 |
| | ventilation | |
| | Ventilation + additional organ | 7 |
| | support - pressors, RRT, | |
| | ECMO | |
| Dead | Death | 8 |

### Biomarkers:

Biomarker samples will be collected at baseline, day 4 and day 7. A small panel of biomarkers will be conducted on all subjects at these time points. Additional biomarkers may be measured based on the results of the trial.

### Biomarkers to be Measured on all Subjects:

Specific biomarkers have been associated with progression and poor outcome following infection with COVID-19. These include differential white cell count, neutrophil to lymphocyte ratio, CRP, IL-6, IL-8, Ferritin, D-Dimer, and Troponin levels. These will be measured in all subjects at baseline, day 4 and day 7.

### Additional Plasma Biomarkers:

Additional plasma biomarkers may be analyzed subject to the clinical data in the trial. These biomarkers may help understand the response to Prevotella histicola Strain B smEVs and/or the progression of COVID-19 disease. These markers could include Eotaxin, Eotaxin-3, GM-CSF, IFN-γ, IL-1α, IL-1β, IL-2, IL-4, IL-5, IL-7, IL-8 (HA), IL-1β, IL-12/IL-23p40, IL-12p70, IL-13, IL-15, IL-16, IL-17A, IP-10, MCP-1, MCP-4, MDC, MIP-1α, MIP-10, TARC, TNF-α, TNF-0, VEGF-A. Additional plasma biomarkers may be analyzed if emerging data suggests they could be useful in understanding the drug response and/or disease progression.

### Transcription Analysis:

RNA will be collected from PBMCs and may be analyzed subject to the clinical data in the trial. The exact genes to be analyzed will be defined by an expert subgroup of the study but will include genes related to host immune response as well as those related to the disease pathology.

### Microbiome Research:

The microbiome composition of stool samples will be assessed as an optional research test at baseline and day 7. Prevotella histicola Strain B smEVs are not expected to alter the composition of the microbiome, but the microbiome will be evaluated for separate research purposes. Microbiome analysis may be performed through 16s ribosomal RNA sequencing and/or whole genome microbial sequencing depending on the question being asked.

### REFERENCES

Cookson C. UK's original coronavirus plan risked ‘hundreds of thousands’ dead. Financial Times. 16 Mar. 2020.
Fine J P and Gray R J. A Proportional Hazards Model for the Subdistribution of a Competing Risk. J Amer Stat Assn, vol. 94, no. 446, 1999, pp. 496-509.
Gralinski L E, and Baric R S. Molecular pathology of emerging coronavirus infections. J Pathol, 2015: 235: 185-195.
Guan W J, Ni Z Y, Hu Y et al. Clinical Characteristics of Coronavirus Disease 2019 in China. NEJM, 2020: 382(18):1708-1720.
Hagau N, Slavcovici A, Gonganau D N et al. Clinical aspects and cytokine response in severe H1N1 influenza A virus infection. Crit Care, 2010: 14(6) R203.
Huang C, Wang Y, Li X et al. Clinical features of patients infected with 2019 novel coronavirus in Wuhan, China. Lancet. 2020; 395(10223):497-506.
de Jong M D, Simmons C P, Thanh T T et al. Fatal outcome of human influenza A (H5N1) is associated with high viral load and hypercytokinemia. Nat Med, October 2006: 12(10) 1203-7.
Maharaj R. King's Critical Care—Evidence Summary Clinical Management of COVID-19. King's Critical Care. 9 Mar. 2020: 1-24.
Liu Y, Yang Y, Zhang C et al. Clinical and biochemical indexes from 2019-nCoV infected patients linked to viral loads and lung injury. Sci. China Life Sci. 2020: 63, 364-374.
Mehta P, McAuley D F, Brown M et al. Covid-19: consider cytokine storm syndromes and immunosuppression; The Lancet. 2020: 395, 1033-4.
Peiris J S, Chu C M, Cheng V C et al. Clinical progression and viral load in a community outbreak of coronavirus-associated SARS pneumonia: a prospective study. Lancet. 2003: 361: 1767-72.
Ruan Q, Yang K, Wang W et al. Clinical predictors of mortality due to COVID-19 based on an analysis of data of 150 patients from Wuhan, China. Intensive Care Med. 3 Mar. 2020.
Villar J, Pérez-Méndez L, Blanco J, et al. Spanish Initiative for Epidemiology, Stratification, and Therapies for ARDS (SIESTA) Network. A universal definition of ARDS: the PaO2/FiO2 ratio under a standard ventilatory setting—a prospective, multicenter validation study. Intensive Care Med. 2013 April; 39(4):583-92.
Wang D. Hu B, Hu C et al. Clinical Characteristics of 138 Hospitalized Patients With 2019 Novel Coronavirus-Infected Pneumonia in Wuhan, China. JAMA. 2020: 323, 1061-1069.
Wong C K, Lam C W K, Wu A K L et al. Plasma inflammatory cytokines and chemokines in severe acute respiratory syndrome. Clin Exp Immunol. 2004: 136, 95-103.

### INCORPORATION BY REFERENCE

All publications and patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a viral infection in a human subject in need thereof, the method comprising administering to the subject an effective dose of extracellular vesicles (EVs) from a Prevotella histicola strain and/or a composition comprising extracellular vesicles (EVs) from a Prevotella histicola strain.

2. A method of treating COVID-19 in a human subject in need thereof, the method comprising administering to the subject an effective dose of extracellular vesicles (EVs) from a Prevotella histicola strain and/or a composition comprising extracellular vesicles (EVs) from a Prevotella histicola strain.

3. A method of reducing IL-8, IL-6, IL-1β, and/or TNFα expression levels in a human subject in need thereof, the method comprising administering to the subject an effective dose of extracellular vesicles (EVs) from a Prevotella histicola strain and/or a composition comprising extracellular vesicles (EVs) from a Prevotella histicola strain.

4. The method of any one of claims 1 to 3, wherein the extracellular vesicles are from a Prevotella histicola strain comprising at least 95%, at least 96%, at least 97%, at least 98%, at least 99% sequence identity, at least 99.5% sequence identity, at least 99.6% sequence identity, at least 99.7% sequence identity, at least 99.8% sequence identity, or at least 99.9% sequence identity to the genomic sequence, 16S sequence, and/or CRISPR sequence, of Prevotella histicola Strain B (NRRL accession number B 50329).

5. The method of any one of claims 1 to 4, wherein the Prevotella histicola strain is Prevotella Strain B (NRRL accession number B 50329).

6. The method of any one of claims 1 to 5, wherein the EVs are administered orally.

7. The method of any one of claims 1 to 6, wherein the dose is in the form of one or more capsules, optionally wherein the one or more capsules comprise an enteric-coating.

8. The method of any one of claims 1 to 6, wherein the dose is in the form of one or more tablets, optionally wherein the one or more tablets comprise an enteric-coating.

9. The method of any one of claims 1 to 6, wherein the dose is in the form of one or more mini-tablets.

10. The method of claim 15, wherein the mini-tablets are enteric-coated mini-tablets.

11. The method of any one of claims 1 to 6, wherein the dose is in the form of a non-enteric coated capsule comprising an enteric-coated mini-tablet.

12. The method of any one of claims 1 to 17, wherein the dose is administered in combination with an additional therapeutic agent or an additional therapy.

13. A solution comprising extracellular vesicles (EVs) from a Prevotella histicola strain and an excipient that comprises a bulking agent.

14. A therapeutic composition comprising the solution of claim 13, wherein the composition further comprises a pharmaceutically acceptable excipient.

15. The therapeutic composition of claim 14, wherein the pharmaceutically acceptable excipient comprises a glidant, lubricant, and/or diluent.

16. A powder comprising extracellular vesicles (EVs) from a Prevotella histicola strain and an excipient that comprises a bulking agent.

17. A therapeutic composition comprising the powder of claim 16, wherein the composition further comprises a pharmaceutically acceptable excipient.

18. The therapeutic composition of claim 17, wherein the pharmaceutically acceptable excipient comprises a glidant, lubricant, and/or diluent.

19. A therapeutic composition comprising extracellular vesicles (EVs) from a Prevotella histicola strain and an excipient that comprises a bulking agent.

20. A solution comprising extracellular vesicles (EVs) from a Prevotella histicola strain and an excipient stock of a formula provided in Table A, B, C, or D.

21. A therapeutic composition comprising the solution of claim 20, wherein the composition further comprises a pharmaceutically acceptable excipient.

22. The therapeutic composition of claim 21, wherein the pharmaceutically acceptable excipient comprises a glidant, lubricant, and/or diluent.

23. A powder comprising extracellular vesicles (EVs) from a Prevotella histicola strain and an excipient stock of a formula provided in Table A, B, C, or D.

24. A therapeutic composition comprising the powder of claim 23, wherein the composition further comprises a pharmaceutically acceptable excipient.

25. The therapeutic composition of claim 24, wherein the pharmaceutically acceptable excipient comprises a glidant, lubricant, and/or diluent.

26. A method of treating a human subject in need thereof, the method comprising:
administering to the subject a therapeutically effective amount of the solution, powder, or therapeutic composition of any one of claims 13 to 25.

27. The solution, powder, or therapeutic composition of any one of claims 13 to 25 for use in treating a human subject in need thereof.

28. Use of the solution, powder, or therapeutic composition of any one of claims 13 to 25 for the preparation of a medicament for treating a human subject subject in need thereof.

29. The method/solution/powder/therapeutic composition/use of any one of claims 26 to 28, wherein the solution/powder/therapeutic composition is orally administered.

30. The method/solution/powder/therapeutic composition/use of any one of claims 26 to 28, wherein the subject is in need of treatment and/or prevention of coronavirus infection, an influenza virus infection, and/or a respiratory syncytial virus infection.

31. The method/solution/powder/therapeutic composition/use of any one of claims 26 to 28, wherein the subject is in need of treatment and/or prevention of SARS-CoV-2 infection.

32. The method/solution/powder/therapeutic composition/use of any one of claims 26 to 31, wherein the solution/powder/therapeutic composition is administered in combination with an additional therapeutic agent or an additional therapy.

33. The method/solution/powder/therapeutic composition/use of any one of claims 26 to 32, wherein the extracellular vesicles are from a Prevotella histicola strain comprising at least 95%, at least 96%, at least 97%, at least 98%, at least 99% sequence identity, at least 99.5% sequence identity, at least 99.6% sequence identity, at least 99.7% sequence identity, at least 99.8% sequence identity, or at least 99.9% sequence identity to the nucleotide sequence genomic sequence, 16S sequence, and/or CRISPR sequence, of Prevotella histicola Strain B (NRRL accession number B 50329.

34. The method/solution/powder/therapeutic composition/use of any one of claims 26 to 33, wherein the Prevotella histicola strain is Prevotella histicola Strain B (NRRL accession number B 50329).

35. The method of any one of claims 1 to 34, wherein the subject has been infected with a coronavirus, an influenza virus, and/or a respiratory syncytial virus.

36. The method of any one of claims 1 to 35, wherein the subject has been infected with SARS-CoV-2.

37. The method of claim 36, wherein the subject has COVID-19.

38. The method of any one of claims 1 to 37, wherein the subject is traveling to a region where SARS-CoV-2 infection is endemic.

39. The method of any one of claims 1 to 38, wherein the subject has been exposed to a source infected with a coronavirus, an influenza virus, and/or a respiratory syncytial virus.

40. The method of any one of claims 1 to 39, wherein the subject has been exposed to a source infected with SARS-CoV-2.

41. The method of claim any one of claims 1 to 40, further comprising administering to the subject an antiviral medication.

42. The method of claim 41, wherein the antiviral medication is ribavirin, neuraminidase inhibitor, protease inhibitor, recombinant interferons, antibodies, oseltamivir, zanamivir, peramivir or baloxavir marboxil.

43. The method of any one of claims 1 to 42, further comprising administering to the subject an anti-inflammatory agent.

44. The method of claim 43, wherein the anti-inflammatory agent is an NSAID or an anti-inflammatory steroid.

45. The method of claim any one of claims 1 to 44, further comprising administering to the subject hydroxychloroquine, chloroquine, remdesivir, tocilizumab and/or sarilumab.

46. A method of identifying a subject as being at risk for increased severity of a disease or condition, the method comprising determining expression levels IL-8, IL-6, IL-1β, and/or TNFα in a sample from the subject, wherein elevated expression levels of IL-8, IL-6, IL-1β, and/or TNFα in the sample indicate that the subject is at of increased severity of the disease or condition.

47. The method of claim 46, wherein the disease or condition is a coronavirus infection, an influenza virus infection, and/or a respiratory syncytial virus infection.

48. The method of claim 46, wherein the disease or condition is SARS-CoV-2 infection.

49. The method of any one of claims 46 to 48, further comprising treating the subject for the disease or condition.

50. The method of claim 49, wherein the treatment comprises orally administering to the subject a therapeutically effective dose of extracellular vesicles (EVs) from a Prevotella histicola strain and/or a composition comprising the extracellular vesicles, optionally wherein the composition is a solution, powder and/or therapeutic composition.

51. The method of claim 50, wherein the extracellular vesicles are from a Prevotella histicola strain comprising at least 95%, at least 96%, at least 97%, at least 98%, at least 99% sequence identity, at least 99.5% sequence identity, at least 99.6% sequence identity, at least 99.7% sequence identity, at least 99.8% sequence identity, at least 99.9% sequence identity to the genomic sequence, 16S sequence, and/or CRISPR sequence of Prevotella histicola Strain B (NRRL accession number B 50329).

52. The method of claim 50, wherein the Prevotella histicola strain is Prevotella Strain B (NRRL accession number B 50329).

53. The method of any one of claims 49 to 52, wherein the treatment comprises administering the subject an antiviral medication.

54. The method of claim 53, wherein the antiviral medication is ribavirin, neuraminidase inhibitor, protease inhibitor, recombinant interferons, antibodies, oseltamivir, zanamivir, peramivir or baloxavir marboxil.

55. The method of any one of claims 49 to 54, wherein the treatment comprises administering the subject an anti-inflammatory agent.

56. The method of claim 55, wherein the anti-inflammatory agent is an NSAID or an anti-inflammatory steroid.
